(19) United States
(12) Patent Application Publication
(10) Pub. No.: US 2019/0110740 A1
(43) Pub. Date: Apr. 18, 2019

(54) SYSTEM, APPARATUS AND METHOD FOR ASSESSING WOUND AND TISSUE CONDITIONS

(71) Applicants: James G. Spahn, Carmel, IN (US); James D. Spahn, Carmel, IN (US); Todd J. Pickard, Carmel, IN (US)

(72) Inventors: James G. Spahn, Carmel, IN (US); James D. Spahn, Carmel, IN (US); Todd J. Pickard, Carmel, IN (US)

(21) Appl. No.: 16/153,624

(22) Filed: Oct. 5, 2018

Related U.S. Application Data
(63) Continuation of application No. 15/787,707, filed on Oct. 19, 2017, now abandoned.
Application No. 15/787,707 is a continuation-in-part of application No. 14/984,346, filed on Dec. 30, 2015, now Pat. No. 10,169,860.
(60) Provisional application No. 62/410,033, filed on Oct. 19, 2016.
(60) Provisional application No. 62/410,117, filed on Oct. 19, 2016.
(60) Provisional application No. 62/410,150, filed on Oct. 19, 2016.

(51) Int. Cl. A61B 5/00; G06T 7/62; A61B 5/01; G01J 5/02; G06T 7/00; G01J 5/00; G01J 5/08

(52) U.S. Cl. CPC A61B 5/445; A61B 5/1072; A61B 5/015; A61B 5/0013; A61B 5/7425; A61B 5/441; G01J 5/025; A61B 5/01; G06T 7/0012; G01J 5/0025; G01J 5/089; G01J 5/0859; A61B 5/0077; A61B 5/0075; G01J 5/0265; A61B 5/6844; G06T 7/62

(57) ABSTRACT

A combination thermal and visual image capturing device used to capture real time thermal and visual images of surface and subsurface biological tissue, said device comprising: a power source, said power source functionally connected to said device; a housing, a long wave infrared microbolometer, said microbolometer functionally connected to said power source a digital camera; a short wave infrared microbolometer, said microbolometer functionally connected to said power source; a digital camera, said digital camera functionally connected to said power source; and a digital camera, said digital camera functionally connected to said power source, wherein said digital camera and 3D camera are contained within a USB peripheral device; said imaging apparatus further comprising means to electronically provide combined thermal image information from the microbolometers and visual image information from said digital camera and said 3D camera to another electronic device.

### CROSS REFERENCE TO RELATED APPLICATIONS

This application is a continuation-in-part of co-pending U.S. patent application Ser. No. 14/984,346 filed Dec. 30, 2015, which is a division of U.S. patent application Ser. No. 13/439,177, filed Apr. 4, 2012 (now U.S. Pat. No. 9,357,963), which claims priority to U.S. Provisional Patent Application Ser. No. 61/516,459 filed Apr. 4, 2011; and this application claims priority to U.S. Provisional Patent Application Ser. Nos. 62/409,663 and 62/409,700 both filed Oct. 18, 2016, and to U.S. Provisional Patent Applications Ser. Nos. 62/410,033, 62/410,117 and 62/410,150 all filed Oct. 19, 2016.

### BACKGROUND

### 1. Field of the Invention

The present invention relates to diagnostic medical imaging and, more particularly, to three dimensional (“3D”) and thermographic imaging for use with the treatment of wounds.

### 2. Description of the Related Art

Over the last century, clinicians, which term includes herein certified and licensed medical doctors of all specialties, osteopathic doctors of all specialties, podiatrists, dental doctors of all specialties, chiropractors, veterinarians of all specialties, nurses, and medical imaging technicians, have become dependent on the use of medical devices that assist them in their delivery of patient-centered care. The common function of these devices is to assist and not replace the clinical judgment of the clinician. This fulfills the dictum that best practice is clinical judgment assisted by scientific data and information.

Entering into the era of computer science and sophisticated electronics, clinicians have the opportunity to be supported by data and information in a statistically significant and timely fashion. These advancements have allowed more extensive and useful collection of meaningful data that can be acquired, analyzed, and applied in conjunction with the knowledge and expertise of the clinician.

Medical long-wave infrared (LIR) thermography has been known to be beneficial in the evaluation of thermal heat intensity and gradiency relating to abnormalities of the skin and subcutaneous tissue (SST). Although this technology has expanded to other areas of medical evaluation, the scope of this patent application is limited to the SST abnormalities. These abnormalities include the formation of deep tissue injury (DTI) and subsequent necrosis caused by mechanical stress, infection, auto-immune condition, and vascular flow problems. DTI caused by mechanical stress (pressure, shear and frictional forces) can be separated into three categories. The first category is a high magnitude/short duration mechanical stress represented by traumatic and surgical wounds. The second category is low magnitude/long duration mechanical stress represented by pressure ulcer development, which is also a factor in the development of ischemic and neuropathic wounds. The third category is a combination of categories one and two represented by pressure ulcer formation in the bariatric patient.

The pathophysiologic conditions that occur with DTI and subsequent necrosis of the affected tissue are ischemia, cell distortion, impaired lymphatic drainage, impaired interstitial fluid flow, and reperfusion injury: Category one is dominated by cell distortion and even destruction. Category two is dominated by ischemia. Category three is a combination of cell distortion and ischemia.

Hypoxia causes aerobic metabolism to convert to anaerobic metabolism. This occurrence causes lactic acidosis followed by cell destruction, release of enzymes and lytic reactions. The release of these substances causes additional cell injury and destruction, and initiation of the inflammatory response.

It is very important to recognize that ischemic-reperfusion injury is associated with all of the above mechanical stress induced SST injuries. This condition is caused by a hypoxia induced enzymatic change and the respiratory burst associated with phagocytosis when oxygen returns after an ischemic event. The result of ischemic-reperfusion injury is the formation of oxygen free radicals (hydroxyl, superoxide, and hydrogen peroxide) that cause damage to healthy and already injured cells leading to extension of the original injury.

SST injury and subsequent necrosis can also be caused by vascular disorders. Hypoxia can be caused by an arterial occlusion or by venous hypertension. Lymphatic flow or node obstruction can also create vascular induced injury by creating fibrous restriction to venous drainage and subsequent cellular stasis in the capillary system. These disorders are also accentuated by reperfusion injury and oxygen free radical formation.

Infection of the skin (impetigo), subcutaneous tissue (cellulitis), deep tissue (fasciitis), bone (osteomyelitis) and cartilage (chondritis) causes injury and necrosis of the affected tissue. Cells can be injured or destroyed by the microorganism directly, by toxins released by the microorganism and/or the subsequent immune and inflammatory response. These disorders are also accentuated by reperfusion injury and oxygen free radical formation.

Auto-immune morbidities of the skeletal joints (rheumatoid arthritis), subcutaneous tissue (tendonitis, myelitis, dermatitis) and blood vessels (vasculitis) cause similar dysfunction and necrosis of the tissue being affected by the hypersensitivity reactions on the targeted cells and the subsequent inflammatory response. Again, these conditions are accentuated by reperfusion and oxygen free radical formation.

The common event that addresses all of the above SST injuries is the inflammatory response. This response has two stages. The first stage is vascular and the second is cellular. The initial vascular response is vasoconstriction that will last a short time. The constriction causes decreased blood flow to the area of injury. The decrease in blood flow causes vascular “pooling” of blood (passive congestion) in the proximal arterial vasculature in the region of injury and intravascular cellular stasis occurs along with coagulation.

The second vascular response is extensive vasodilation of the blood vessels in the area of necrosis. This dilation along with the “pooled” proximal blood causes increased blood flow with high perfusion pressure into the area of injury. This high pressure flow can cause damage to endothelial cells. Leakage of plasma, protein, and intravascular cells causes more cellular stasis in the capillaries (micro-thrombotic event) and hemorrhage into the area of injury. When the perivascular collagen is injured, intravascular and extravascular coagulation occurs. The rupture of the mast cells causes release of histamine that increases the vascular dilation and the size of the junctions between the endothelial cells. This is the beginning of the cellular phase. More serum and cells (mainly neutrophils) enter into the area of the mixture of injured and destroyed cells by the mechanism of marginalization, emigration (diapedesis) and the chemotaxic recruitment (chemotaxic gradiency). Stalling of the inflammatory stage can cause the area of necrosis (ring of ischemia) to remain in the inflammatory stage long past the anticipated time of 2-4 days. This continuation of the inflammatory stage leads to delayed resolution of the ischemic necrotic event.

The proliferation stage starts before the inflammatory stage recedes. In this stage angiogenesis occurs along with formation of granulation and collagen deposition. Contraction occurs, and peaks, at 5-15 days post injury.

Re-epithelialization occurs by various processes depending on the depth of injury. Partial thickness wounds can resurface within a few days. Full thickness wounds need granulation tissue to form the base for re-epithelialization to occur. The full thickness wound does not heal by regeneration due to the need for scar tissue to repair the wound. The repaired scarred wound has less vascularity and tensile strength of normal regional uninjured SST. The final stage is remodeling. In this stage the collagen changes from type III to a stronger type I and is rearranged into an organized tissue.

All stages of wound healing require adequate vascularization to prevent ischemia, deliver nutrients, and remove metabolic waste. Following the vascular flow and metabolic activity of a necrotic area is currently monitored by patient assessment and clinical findings of swelling, pain, redness, increased temperature, and loss of function.

Having a real-time control allows an area of interest (AOI) to be recognized. The AOI can be of greater intensity (hotter) or less intensity (cooler) than the normal SST of that region of the body. The AOI can then be evaluated by the clinician for the degree of metabolism, blood flow, necrosis, inflammation and the presence of infection by comparing the warmer or cooler thermal intensity of the AOI or wound base and peri-AOI or wound area to the normal SST of the location being imaged. Serial imaging also can assist the clinician in the ability to recognize improvement or regression of the AOI or wound over time.

The use of an LIR thermal and digital visual imager can be a useful adjunct tool for clinicians with appropriate training to be able to recognize physiologic and anatomical changes in an AOI before it presents clinically and also the status of the AOI/wound in a trending format. By combining the knowledge obtained from the images with a comprehensive assessment, skin and subcutaneous tissue evaluation, and an AOI or wound evaluation will assist the clinician in analyzing the etiology, improvement or deterioration, and the presence of infection affecting the AOI or wound.

The foundational scientific principles behind LIR thermography technology are energy, heat, temperature, and metabolism.

Energy is not a stand-alone concept. Energy can be passed from one system to another, and can change from one form to another, but can never be lost. This is the First Law of Thermodynamics. Energy is an attribute of matter and electromagnetic radiation. It is observed and/or measured only indirectly through effects on matter that acquires, loses or possesses it and it comes in many forms such as mechanical, chemical, electrical, radiation (light), and thermal.

The present application focuses on thermal and chemical energy. Thermal energy is the sum of all of the microscopic scale randomized kinetic energy within a body, which is mostly kinetic energy. Chemical energy is the energy of electrons in the force field created by two or more nuclei; mostly potential energy.

Energy is transferred by the process of heat. Heat is a process in which thermal energy enters or leaves a body as the result of a temperature difference. Heat is therefore the transfer of energy due to a difference in temperature; heat is a process and only exists when it is flowing. When there is a temperature difference between two objects or two areas within the same object, heat transfer occurs. Heat energy transfers from the warmer areas to the cooler areas until thermal equilibrium is reached. This is the Second Law of Thermodynamics. There are four modes of heat transfer: evaporation, radiation, conduction and convection.

Molecules are the workhorses and are both vehicles for storing and transporting energy and the means of converting it from one form to another. When the formation, breaking, or rearrangement of the chemical bonds within the molecules is accompanied by the uptake or release of energy it is usually in the form of heat. Work is completely convertible to heat and defined as a transfer due to a difference in temperature, however work is the transfer of energy by any process other than heat. In other words, performance of work involves a transformation of energy.

Temperature measures the average randomized motion of molecules (kinetic energy) in a body. Temperature is an intensive property by which thermal energy manifests itself. It is measured by observing its effect on some temperature dependent variable on matter (i.e. ice/steam points of water). Scales are needed to express temperature numerically and are marked off in uniform increments (degrees).

As a body loses or gains heat, its temperature changes in direct proportion to the amount of thermal energy transferred from a high temperature object to a lower temperature object. Skin temperature rises and falls with the temperature of the surroundings. This is the temperature that is referred to in reference to the skins ability to lose heat its surroundings.

The temperature of the deep tissues of the body (core temperatures) remains constant (within ±1° F. or ±0.6° C.) unless the person develops a febrile illness. No single temperature can be considered normal. Temperature measurements on people who had no illness have shown a range of normal temperatures. The average core temperature is generally considered to be between 98.0° F. and 98.6° F. measured orally or 99.0° F. and 99.6° F. measured rectally. The body can temporarily tolerate a temperature as high as 101° F. to 104° F. (38.6° C. to 40° C.) and as low as 96° F. (35.5° C.) or lower.

Metabolism simply means all of the chemical reactions in all of the cells of the body. Metabolism creates thermal energy. The metabolic rate is expressed in terms to the rate of heat release during the chemical reactions. Essentially all the energy expended by the body is eventually converted into heat.

Since heat flows from hot to cold temperature and the body needs to maintain a core temperature of 37.0° C.±0.75° C., the heat is conserved or dissipated to the surroundings. The core heat is moved to the skin surface by blood flow. Decreased flow to the skin surface helps conserve heat, while increased flow promotes dissipation. Conduction of the core heat to the skin surface is fast, but inadequate alone to maintain the core temperature. Heat dissipation from the skin surface (3 mm microclimate) also occurs due to the conduction, convection and evaporation.

Heat production is the principal by-product of metabolism. The rate of heat production is called the metabolic rate of the body. The important factors that affect the metabolic rate are:

1. Basal Rate of Metabolism (ROM) of all cells of the body;

2. Extra ROM caused by muscle activity including shivering;

3. Extra ROM caused by the effect of thyroxine and other hormones to a less extent (i.e.: growth hormone, testosterone);

4. Extra ROM caused by the effect of epinephrine, norepinephrine, and sympathetic stimulation on the cells; and

5. Extra ROM caused by increased chemical activity in the cells themselves, especially when the cell temperature increases.

Most of the heat produced in the body is generated in the deep organs (liver, brain, heart and the skeletal muscles during exercise). The heat is then transferred to the skin where the heat is lost to the air and other structures. The rate that heat is lost is determined by how fast heat can be conducted from where it is produced in the body core to the skin.

The skin, subcutaneous tissues and especially adipose tissue are the heat insulators for the body. The adipose tissue is important since it conducts heat only 33% as effective as other tissue and specifically 52% as effective as muscle. Conduction rate of heat in human tissue is 18 kcal/cm/m2k. The subcutaneous tissue insulator system allows the core temperature to be maintained yet allowing the temperature of the skin to approach the temperature of the surroundings.

Blood flows to the skin from the body core in the following manner. Blood vessels penetrate the adipose tissue and enter a vascular network immediately below the skin. This is where the venous plexus comes into play. The venous plexus is especially important because it is supplied by inflow from the skin capillaries and in certain exposed areas of the body (hands-feet-ears) by the highly muscular arterio-venous anastomosis. Blood flow can vary in the venous plexus from barely above zero to 30% of the total cardiac output. There is an approximate eightfold increase in heat conductance between the fully vasoconstricted state and the fully vasodilated state. The skin is an effective controlled heat radiator system and the controlled flow of blood to the skin is the body's most effective mechanism of heat transfer from the core to the surface.

Heat exchange is based on the scientific principle that heat flows from warmer to cooler temperatures. Temperature is thought of as heat intensity of an object. The methods of heat exchange are: radiation (60%), loss of heat in the form of LIR waves (thermal energy), conduction to a solid object (3%), transfer of heat between objects in direct contact and loss of heat by conduction to air (15%) caused by the transfer of heat, caused by the kinetic energy of molecular motion. Much of this motion can be transferred to the air if it is cooler than the surface. This process is self-limited unless the air moves away from the body. If that happens, there is a loss of heat by convection. Convection is caused by air currents. A small amount of convection always occurs due to warmer air rising. The process of convection is enhanced by any process that moves air more rapidly across the body surface (forced convection). This includes fans, air flow beds and air warming blankets.

Convection can also be caused by a loss of heat by evaporation which is a necessary mechanism at very high air temperatures. Heat (thermal energy) can be lost by radiation and conduction to the surroundings as long as the skin is hotter than the surroundings. When the surrounding temperature is higher than the skin temperature, the body gains heat by both radiation and conduction. Under these hot surrounding conditions the only way the body can release heat is by evaporation. Evaporation occurs when the water molecule absorbs enough heat to change to gas. Due to the fact water molecules absorb a large amount of heat in order to change into a gas, large amounts of body heat can be removed from the body.

Insensible heat loss dissipates the body's heat and is not subject to body temperature control (water loss through the lungs, mouth and skin). This accounts for 10% heat loss produced by the body's basal heat production. Sensible heat loss by evaporation occurs when the body temperature rises, and sweating occurs. Sweating increases the amount of water to the skins surface for vaporization. Sensible heat loss can exceed insensible heat loss by 30 times. The sweating is caused by electrical or excess heat stimulation of the anterior hypothalamus pre-optic area.

The role of the hypothalamus (anterior pre-optic area) in the regulation of the body's temperatures occurs due to nervous feedback mechanisms that determine when the body temperature is either too hot or too cold.

The role of temperature receptors in the skin and deep body tissues relate to cold and warm sensors in the skin. Cold sensors outnumber warm sensors 10 to 1. The deep tissue receptors occur mainly in the spinal cord, abdominal viscera and both in and around the great veins. The deep receptors mainly detect cold rather than warmth. These receptors function to prevent low body temperature. These receptors contribute to body thermoregulation through the bilateral posterior hypothalamus area. This is where the signals from the pre-optic area and the skin and deep tissue sensors are combined to control the heat producing and heat conserving reactions of the body.

Temperature Decreasing Mechanisms:

1. Vasodilation of all blood vessels, but with intense dilation of skin blood vessels that can increase the rate of heat transfer to the skin eight-fold;

2. Sweating can remove 10 times the basal rate of body heat with an additional 1° C. increase in body temperature; and

3. Decrease in heat production by inhibiting shivering and chemical thermogenesis.

Temperature Increasing Mechanisms:

1. Skin vasoconstriction throughout the body; and

2. Increase in heat production by increasing metabolic activity
a. Shivering
   i. 4 to 5 times increase, and
b. Chemical Thermogenesis (brown fat)
   i. Adults 10-15% increase
   ii. Infants 100% increase.

LIR thermography evaluates the infra-red thermal intensity. The microbolometer is a 320×240 pixel array sensor that can acquire the long-wave infrared wavelength (7-14 μm or micron) (NOT near-infrared thermography) and convert the thermal intensity into electrical resistance. The resistance is measured and processed into digital values between 1-254. A digital value represents the long-wave infrared thermal intensity for each of the 76,800 pixels. A grayscale tone is then assigned to the 1-254 thermal intensity digital values. This allows a grayscale image to be developed.

An LIR camera has the ability to detect and display the LIR wavelength in the electromagnetic spectrum. The basis for infrared imaging technology is that any object whose temperature is above 0° K radiates infrared energy. Even very cold objects radiate some infrared energy. Even though the object might be absorbing thermal energy to warm itself, it will still emit some infrared energy that is detectable by sensors. The amount of radiated energy is a function of the object's temperature and its relative efficiency of thermal radiation, known as emissivity.

Emissivity is a measure of a surface's efficiency in transferring infrared energy. It is the ratio of thermal energy emitted by a surface to the energy emitted by a perfect blackbody at the same temperature.

Using LIR thermography is a beneficial device to monitor metabolism and blood flow in a non-invasive test that can be performed bedside with minimal patient and ambient surrounding preparation. The ability to accurately measure the LIR thermal intensity of the human body is made possible because of the skins emissivity (0.98±0.01), which is independent of pigmentation, absorptivity (0.98±0.01), reflectivity (0.02) and transmitability (0.000). The human skin mimics the “Black Body” radiation concept. A perfect blackbody only exists in theory and is an object that absorbs and reemits all of its energy. Human skin is nearly a perfect blackbody as it has an emissivity of 0.98, regardless of actual skin color. These same properties allow temperature degrees to be assigned to the pixel digital value. This is accomplished by calibration utilizing a “Black Body” simulator and an algorithm to account for the above factors plus ambient temperatures. A multi-color palate can be developed by clustering pixel values. There are no industry standards how this should be done so many color presentations are being used by various manufacturers. The use of gray tone values is standardized, consistent and reproducible. Black is considered cold and white is considered hot by the industry.

LIR thermography is a beneficial device to monitor metabolism and blood flow in a non-invasive test that can be performed bedside with minimal patient and ambient surrounding preparation. It uses the scientific principles of energy, heat, temperature and metabolism. Through measurement and interpretation of thermal energy, it produces images that will assist clinicians to make a significant impact on wound care (prevention, early intervention and treatment) through detection.

U.S. Pat. No. 5,803,082 discloses an omnidirectional, multispectral and multimodal sensor/display processor for the screening, examination, detection, and diagnosis of breast cancer. Its capabilities are accomplished through stable vision fusion of the Doppler-like differences of selective radiologic wavelengths, besides X-ray mammograms, e.g., ultraviolet (UV.), visible and infrared (IR), with-vision-computer discrimination of other active and passive observables of electromagnetic fields, and medical data, including the optimum color ratios and 3-dimensional (3D) transformation of multiple imaging modalities, e.g., ultrasound, nuclear computed tomography (CT), magnetic resonance imaging (MRI), etc., to obtain the “concurrence of evidence” necessary for maximum confidence levels, generated at minimal cost and with minimum false positives, at the earliest possible breast cancer detection point.

U.S. Pat. No. 6,775,397 discloses a user recognition system that utilizes two CCD cameras to obtain two images of the user from two different angles of view. A three-dimensional model of the user's face is created from the obtained images in addition. The generated model and an additional facial texture image of the user are compared with a stored user profile. When the obtained 3D model and facial texture information matches the stored profile of the user, access is granted to the system.

U.S. Pat. No. 7,365,330 discloses a computer-implemented method for automated thermal computed tomography includes providing an input of heat, for example, with a flash lamp, onto the surface of a sample. The amount of heat and the temperature rise necessary are dependent on the thermal conductivity and the thickness of the sample being inspected. An infrared camera takes a rapid series of thermal images of the surface of the article, at a selected rate, which can vary from 100 to 2000 frames per second. Each infrared frame tracks the thermal energy as it passes from the surface through the material. Once the infrared data is collected, a data acquisition and control computer processes the collected infrared data to form a three-dimensional (3D) thermal effusivity image.

U.S. Pat. No. 7,436,988 discloses an approach for automatic human face authentication. Taking a 3D triangular facial mesh as input, the approach first automatically extracts the bilateral symmetry plane of the face surface. The intersection between the symmetry plane and the facial surface, namely the Symmetry Profile, is then computed. By using both the mean curvature plot of the facial surface and the curvature plot of the symmetry profile curve, three essential points of the nose on the symmetry profile are automatically extracted. The three essential points uniquely determine a Face Intrinsic Coordinate System (FICS). Different faces are aligned based on the FICS. The Symmetry Profile, together with two transversal profiles, namely the Forehead Profile and the Cheek Profile compose a compact representation, called the SFC representation, of a 3D face surface. The face authentication and recognition steps are finally performed by comparing the SFC representation of the faces.

U.S. Pat. No. 7,605,924 discloses an inspection system is provided to examine internal structures of a target material. This inspection system combines an ultrasonic inspection system and a thermographic inspection system. The thermographic inspection system is attached to ultrasonic inspection and modified to enable thermographic inspection of target materials at distances compatible with laser ultrasonic inspection. Quantitative information is obtained using depth infrared (IR) imaging on the target material. The IR imaging and laser-ultrasound results are combined and projected on a 3D projection of complex shape composites. The thermographic results complement the laser-ultrasound results and yield information about the target material's internal structure that is more complete and more reliable, especially when the target materials are thin composite parts.

U.S. Pat. No. 7,660,444 discloses a user recognition system that utilizes two CCD cameras to obtain two images of the user from two different angles of view. A three-dimensional model of the user's face is created from the obtained images in addition. The generated model and an additional facial texture image of the user are compared with a stored user profile. When the obtained 3D model and facial texture information matches the stored profile of the user, access is granted to the system.

U.S. Pat. No. 7,995,191 discloses a scannerless 3-D imaging apparatus is disclosed which utilizes an amplitude modulated cw light source to illuminate a field of view containing a target of interest. Backscattered light from the target is passed through one or more loss modulators which are modulated at the same frequency as the light source, but with a phase delay δ which can be fixed or variable. The backscattered light is demodulated by the loss modulator and detected with a CCD, CMOS or focal plane array (FPA) detector to construct a 3-D image of the target. The scannerless 3-D imaging apparatus, which can operate in the eye-safe wavelength region 1.4-1.7 μm and which can be constructed as a flash LADAR, has applications for vehicle collision avoidance, autonomous rendezvous and docking, robotic vision, industrial inspection and measurement, 3-D cameras, and facial recognition.

U.S. Pat. No. 8,090,160 discloses a method and system for 3D-aided-2D face recognition under large pose and illumination variations. The method and system includes enrolling a face of a subject into a gallery database using raw 3D data. The method also includes verifying and/or identifying a target face form data produced by a 2D imagining or scanning device. A statistically derived annotated face model is fitted using a subdivision-based deformable model framework to the raw 3D data. The annotated face model is capable of being smoothly deformed into any face so it acts as a universal facial template. During authentication or identification, only a single 2D image is required. The subject specific fitted annotated face model from the gallery is used to lift a texture of a face from a 2D probe image, and a bidirectional relighting algorithm is employed to change the illumination of the gallery texture to match that of the probe. Then, the relit texture is compared to the gallery texture using a view-dependent complex wavelet structural similarity index metric.

U.S. Pat. No. 8,436,006 discloses a calibrated infrared and range imaging sensors used to produce a true-metric three-dimensional (3D) surface model of any body region within the fields of view of both sensors. Curvilinear surface features in both modalities are caused by internal and external anatomical elements. They are extracted to form 3D Feature Maps that are projected onto the skin surface. Skeletonized Feature Maps define subpixel intersections that serve as anatomical landmarks to aggregate multiple images for models of larger regions of the body, and to transform images into precise standard poses. Features are classified by origin, location, and characteristics to produce annotations that are recorded with the images and feature maps in reference image libraries. The system provides an enabling technology for searchable medical image libraries.

U.S. Pat. No. 8,485,668 discloses a technique for utilizing an infrared illuminator, an infrared camera, and a projector to create a virtual 3D model of a real 3D object in real time for users' interaction with the real 3D object.

U.S. Pat. No. 8,659,698 discloses a structured light 3D scanner consisting of a specially designed fixed pattern projector and a camera with a specially designed image sensor is disclosed. A fixed pattern projector has a single fixed pattern mask of sine-like modulated transparency and three infrared LEDs behind the pattern mask; switching between the LEDs shifts the projected patterns. An image sensor has pixels sensitive in the visual band, for acquisition of conventional image and the pixels sensitive in the infrared band, for the depth acquisition.

U.S. Pat. No. 8,836,756 discloses an apparatus and method for acquiring 3D depth information. The apparatus includes a pattern projection unit, an image acquisition unit, and an operation unit. The pattern projection unit projects light, radiated by an infrared light source, into a space in a form of a pattern. The image acquisition unit acquires an image corresponding to the pattern using at least one camera. The operation unit extracts a pattern from the image, analyzes results of the extraction, and calculates information about a 3D distance between objects existing in the space.

U.S. Pat. No. 9,087,233 discloses a method for identifying a person using a mobile communication device, having a camera unit adapted for recording three-dimensional (3D) images, by recording a 3D image of the person's face using the camera unit, performing face recognition on the 2D image data in the recorded 3D image to determine at least two facial points on the 3D image the of person's face, determining a first distance between the at least two facial points in the 2D image data, determining a second distance between the at least two facial points using the depth data of the recorded 3D image, determining a third distance between the at least two facial points using the first distance and the second distance, and identifying the person by comparing the determined third distance to stored distances in a database, wherein each of the stored distances are associated with a person.

U.S. Pat. No. 9,117,105 discloses a 3D face recognition method based on intermediate frequency information in a geometric image as follows: (1) preprocessing a library and test models of 3D faces, including 3D face area cutting, smoothing processing and point cloud thinning, and discarding the lower portion of the face; (2) mapping the remainder of the face to a 2D grid using grid parameters, and performing linear interpolation on the 3D coordinates of the grid top to acquire the 3D coordinate attributes and generating a geometric image of a 3D face model; (3) performing multi-scale filtering with a multi-scale Haar wavelet filter to extract horizontal, vertical, and diagonal intermediate frequency information image images as invariable facial features; (4) calculating the similarity between the test model and the library set model with a wavelet domain structuring similarity algorithm; and (5) judging the test and library set model models with the maximum similarity belong to the same person.

Needed in the art are an apparatus, system, and method for noninvasively capturing a subdermal thermal and 3D dimensional image of wounds for medical diagnostic purposes. The system should automatically: (1) capture visual and thermal images of a wound; (2) trace the perimeter of the wound; (3) calculate the surface area of the wound; (4) calculate the volume of the wound (or report a maximum or average depth of same); and (4) store the images of and data for later clinical evaluation of the wound at a specific time or over time.

### SUMMARY

One embodiment of a method of and/or apparatus for grayscale digital thermographic imaging of abnormalities of the skin and its subcutaneous tissue provides means for increasing and decreasing pixel value brightness by adding a positive or negative offset to the raw pixel value.

Another embodiment of the method of and/or apparatus for grayscale digital thermographic imaging of abnormalities of the skin and its subcutaneous tissue provides means for defining pixel intensity variations of a long wave infrared image by measuring the thermal intensity ratio of the average of all pixel values from a skin abnormality region to the average of all pixel values from unaffected skin regions.

Another embodiment of the method of and/or apparatus for grayscale digital thermographic imaging of abnormalities of the skin and its subcutaneous tissue provides means for maintaining the separation of a thermographic imager from skin at a set distance by converging two light beams emanating from the imager at a point that is the set distance for the imager to be from skin.

Another embodiment of the method of and/or apparatus for grayscale digital thermographic imaging of abnormalities of the skin and its subcutaneous tissue provides means for obtaining the linear length and width measurements of abnormalities and their square area.

Another embodiment of the method of and/or apparatus for grayscale digital thermographic imaging of abnormalities of the skin and its subcutaneous tissue provides means for highlighting the digital thermographic image of an area of skin to be measured and calculating the area of the highlighted portion of the image in square centimeters by determining the total number of pixels highlighted.

Another embodiment of the method of and/or apparatus for grayscale digital thermographic imaging of abnormalities of the skin and its subcutaneous tissue provides means for encircling an area of interest and generating a histogram of the encircled area to project the distribution of pixel values therein.

Another embodiment of the method of and/or apparatus for grayscale digital thermographic imaging of abnormalities of the skin and its subcutaneous tissue provides means for plotting profile lines in or through an area of skin that is of interest and comparing it with a corresponding profile line of normal skin.

One exemplary embodiment according to the present invention provides a combination thermal and visual image capturing device is provided to capture real time thermal and visual images of surface and subsurface biological tissue. The device is a USB peripheral device that includes: a power source, a housing, a long wave infrared microbolometer functionally connected to the power source, a short wave infrared microbolometer functionally connected to the power source, a 3D camera functionally connected to the power source, and a digital camera functionally connected to the power source. The 3D and digital cameras are contained within the housing. The device further includes means for electronically providing combined thermal image information from the microbolometers and visual image information from the digital and 3D cameras to another electronic device or system.

In another exemplary embodiment, the present invention provides a combination thermal and visual image capturing system used to capture, store, and report combined 2D, 3D, thermal and visual images of surface and subsurface biological tissue. The system includes an image capturing device that is a USB peripheral device including: a power source; a housing; a long wave infrared microbolometer functionally connected to the power source; a digital camera functionally connected to the power source; a short wave infrared microbolometer functionally connected to the power source; and a 3D camera functionally connected to the power source. The digital camera and 3D camera are contained within the housing. The device includes means for combining image data into a single or layered visual image; and means for electronically displaying or storing combined thermal image information from the microbolometers and visual image information from the digital 3D cameras.

In another exemplary embodiment, the present invention provides a method for capturing and combining a long wave infrared image, a short wave infrared image, a 3D image, and a 2D image into a single fused image. The method includes the steps of: obtaining a short wave infrared image; obtaining a long wave infrared image; obtaining a 2D color image; and combining the images into a single fused 3D image.

### BRIEF DESCRIPTION OF THE DRAWINGS

The present invention will be understood more fully from the detailed description given hereinafter and from the accompanying drawings of the preferred embodiment of the present invention, which, however, should not be taken to limit the invention, but are for explanation and understanding only.

In the drawings:

FIG. 1 shows a medical long wave infrared (LIR) and visual views compared;

FIG. 2 shows a thermal span with default configuration settings;

FIG. 3 shows an effect of adding a positive offset of the thermal span;

FIG. 4 shows effect of adding negative offset on the thermal span;

FIG. 5 shows a thermal image of a hand taken with default settings;

FIG. 6 shows a thermal image of the hand when a positive offset is added;

FIG. 7 shows a normal and abnormal selections made from a thermal image and the corresponding results;

FIG. 8 shows an original image (left side) and thermal image (right side-zoomed in) with abnormal selections made;

FIG. 9 shows a schematic representing pixel intensity recognition (zoomed);

FIG. 10 shows a diagram of laser lights implementation;

FIG. 11 shows an experimental setup used to determine digital camera and long-wave infrared microbolometer angels of inclination;

FIG. 12 shows an embodiment of laser lights at an 18-inch distance;

FIG. 13 shows a length and width measurements form an area of interest;

FIG. 14 shows a schematic representing pixel intensity recognition (zoomed);

FIG. 15 shows a periwound region including the wound base highlighted as area of interest and the results obtained for the area selected;

FIG. 16 shows an area including normal, periwound and the wound base regions highlighted as area of interest and the corresponding results obtained for the area selected;

FIG. 17 shows wound histograms;

FIG. 18 shows normal histograms;

FIG. 19 shows a profile line showing the variation in the grayscale values along the line drawn over an area of interest;

FIG. 20 shows comparing the Profile Line with the Reference Line;

FIG. 21 shows a thermal Profile Line;

FIG. 22 shows a figure illustrating the formula for calculating area under the curve;

FIG. 23 shows calculating areas above and below the selected normal;

FIG. 24 shows a profile Line drawn through three fingers;

FIG. 25 shows a profile Line plot on a graph;

FIG. 26 shows the WoundVision Scout device;

FIG. 27 shows a first example wound shape;

FIG. 28 shows a second example wound shape;

FIG. 29 is a graph showing percentage difference from true wound area by measurement methodology;

FIGS. 30A, 30B and 30C respectively show graphs of intended use population, within-reader CV %, for three measurement methodologies;

FIGS. 31A, 31B and 31C respectively show graphs of intended use study, between-reader CV %, 5 readers' average, for three measurement methodologies;

FIGS. 32A, 32B and 32C respectively show graphs of within-reader CV %, clinician average, for three measurement methodologies;

FIG. 33 shows overlaying the wound edge trace from the visual image (on left) onto the thermal image (on right);

FIG. 34 illustrates Step 1 of method to achieve visual-to-thermal overlay;

FIG. 35 illustrates Step 2 of method to achieve visual-to-thermal overlay;

FIG. 36 illustrates Step 3 of method to achieve visual-to-thermal overlay;

FIG. 37 illustrates Step 4 of method to achieve visual-to-thermal overlay;

FIG. 38 illustrates Step 5 of method to achieve visual-to-thermal overlay;

FIG. 39 illustrates Step 6 of method to achieve visual-to-thermal overlay;

FIG. 40 shows a thermal image in raw grayscale pixel value (PV);

FIG. 41 shows a color-filtered pixel value (PV) corresponding to FIG. 40;

FIG. 42 is a graph of within-reader CV % for mean temperature averaged across 5 readers;

FIG. 43 is a graph of between-reader CV % for mean temperature averaged across 5 readers;

FIG. 44A shows a grayscale thermal image (no control) example of an initial patient encounter for control area selection;

FIG. 44B shows a relative color image (control) example of the initial patient encounter for control area selection, corresponding to FIG. 44A;

FIG. 45A-1 shows grayscale thermal image (no control) for example longitudinal encounter #1;

FIG. 45A-2 shows a relative color image (control) for example longitudinal encounter #1 corresponding to FIG. 45A-1;

FIG. 45B-1 shows grayscale thermal image (no control) for example longitudinal encounter #2;

FIG. 45B-2 shows a relative color image (control) for example longitudinal encounter #2 corresponding to FIG. 45B-1;

FIG. 45C-1 shows grayscale thermal image (no control) for example longitudinal encounter #3;

FIG. 45C-2 shows a relative color image (control) for example longitudinal encounter #3 corresponding to FIG. 45C-1;

FIG. 46 is a graph of within-reader CV % for mean temperature averaged across 3 readers;

FIG. 47 is a graph of between-reader CV % for mean temperature averaged across 3 readers;

FIG. 48 is a graph of between-reader CV % for mean temperature averaged across 3 readers;

FIG. 49 is a grapy of within- and between-reader average, max, and min difference in mean temperature for methods 1 and 2;

FIG. 50A is a visual image of a suspected deep tissue injury pre-treatment;

FIG. 50B is a thermal image of the suspected deep tissue injury of FIG. 50A;

FIG. 51A is a visual image of a suspected deep tissue injury of FIG. 50A post-treatment;

FIG. 51B is a thermal image of the suspected deep tissue injury of FIG. 51A;

FIG. 52A is a visual image of a surgical site infection pre-treatment;

FIG. 52B is a thermal image of the surgical site infection of FIG. 52A;

FIG. 53A is a visual image of a surgical site infection of FIG. 52A post-treatment;

FIG. 53B is a thermal image of the surgical site infection of FIG. 53A;

FIG. 54A is a visual image of an amputation site at encounter #1, prior to NPWT;

FIG. 54B is a thermal image of the amputation site of FIG. 54A;

FIG. 55A is a visual image of the amputation site at encounter #2, 5 days after continued NPWT;

FIG. 55B is a thermal image of the amputation site of FIG. 55A;

FIG. 56A is a visual image of the amputation site at encounter #3, 17 days after continued NPWT;

FIG. 56B is a thermal image of the amputation site of FIG. 56A;

FIG. 57A is a visual image of post below the knee amputation;

FIG. 57B is a thermal image corresponding to FIG. 57A;

FIG. 58A is a visual image of post above the knee amputation; and

FIG. 58B is a thermal image corresponding to FIG. 58A.

Corresponding reference characters indicate corresponding parts throughout the several views. The exemplary embodiments set forth herein are not to be construed as limiting the scope of the invention in any manner.

### DETAILED DESCRIPTION OF THE EMBODIMENTS

The present invention will be discussed hereinafter in detail in terms of various exemplary embodiments according to the present invention with reference to the accompanying drawings. In the following detailed description, numerous specific details are set forth in order to provide a thorough understanding of the present invention. It will be obvious, however, to those skilled in the art that the present invention may be practiced without these specific details. In other instances, well-known structures are not shown in detail in order to avoid unnecessary obscuring of the present invention.

Thus, all of the implementations described below are exemplary implementations provided to enable persons skilled in the art to make or use the embodiments of the disclosure and are not intended to limit the scope of the disclosure, which is defined by the claims. As used herein, the word “exemplary” or “illustrative” means “serving as an example, instance, or illustration.”

Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Thermal images taken of the skin surface are constructed by passively reading emitted radiant energy formed by the subcutaneous tissue and the skin tissue by detecting wavelengths in the long-wave infrared range (LIR) of 7-14 μm, and then in real time converting these values into pixels within a digital image. The value assigned to the pixel indicates the thermal intensities of a particular area of the skin when imaged. The thermal images in this embodiment are presented in digital unsigned (not having a plus or minus sign) 8-bit grayscale with pixel values ranging from 0-254, however these same techniques work with images of varying color resolutions. These images could be stored in the data bank along with the information about the data the image has captured so that it can be retrieved by a clinician for future review and analysis. Generally, the unaffected skin thermal intensity will be a uniform gray color within a range of +/−3 to 6-pixel values, which is equal to 0.25 to 0.5 degrees centigrade. Abnormally hot areas of the skin will be represented by patches of increasingly white pixels, while abnormally cold areas will be represented by increasingly dark patches of pixels.

The use of LIR (7-14 μm) imaging along with visual digital imaging allows both physiologic (long-wave infrared and visual) and anatomic assessment of skin and subcutaneous tissue abnormalities and or existing open wounds. The gradiency of the thermal intensity, not the absolute amount of intensity, is the important component of the long-wave thermal image analysis that will allow the clinician to evaluate pathophysiologic events. This capability is beneficial to the clinician in the prevention, early intervention and treatment assessments of a developing existing condition caused by, but not exclusively, wounds, infection, trauma, ischemic events and autoimmune activity.

Utilizing temperature values (° F., ° C., and Kelvin) as the numerical values of LIR thermal heat intensity is complicated due to the need to have a controlled environment. This is required since the value of the temperature scales is affected by ambient temperature, convection of air, and humidity. These variables would need to be measured and documented continuously if temperature values were used. Also the emissivity, absorptivity, reflexivity and transmitability of the skin and subcutaneous tissue can be affected by skin moisture, scabbing, slough and/or eschar formation in an open wound.

To address this problem the imager utilizes the raw data captured by the microbolometer. This data is utilized in determining pixel values relating to the intensity of the thermal energy from the long-wave infrared electromagnetic radiation spectrum being emitted by the human body. The pixel gradient intensifies are represented for visualization by the grayscale presentation.

The pixel values in the grayscale thermal images also vary with the varying conditions mentioned above and hence the algorithms proposed in this application use the average pixel value of the unaffected skin region for that patient on the day the image was taken as a reference point for all the calculations.

Combining the above technique with suggested usage of unaffected skin and subcutaneous tissue in the proximity of an abnormality of a skin/subcutaneous tissue location as a real time control helps to minimize the variability and time consuming requirements in utilizing temperature scales.

There is a difference in the LIR thermal intensity regions of the human body. LIR images have a defined pixel intensity range that is based on the specific usage of an LIR image. In the arena of skin and subcutaneous tissue LIR thermal gradiency, the range is within homeostasis requirements to sustain life. The visualization of pixel intensities is accomplished by the use of a standardized 8-bit grayscale. Black defines cold, gray tones define cool and/or warm and white defines hot. When the imager is used for capturing extremely hot or extremely cold regions that fall outside the thermal range of the imager the pixel values reach the saturation point and it becomes extremely difficult for the human eye to differentiate variations in the pixel values.

This situation can be addressed by utilizing a visualization technique that increases the pixel values to create a positive offset to make the image look brighter. In the same manner a negative offset can be used to decrease the pixel values to make the image look darker.

A. Increasing and Decreasing Pixel Value Brightness by Adding a Positive or Negative Offset to the Raw Pixel Value:

The positive and negative offset can be utilized to assist in visualizing the area of the body being imaged. The usage of the offsets can then be documented as being used at the time the image is initially taken. The default gray tone that represents the actual pixel values is the raw data being stored in the data bank so future analysis can be performed by clinicians at a later time and/or in another location. The default grayscale data is accompanied by documentation of the use of either the positive or negative offset process. This allows for enhanced visualization of black and white extremes in the grayscale image. The goal is to visually enhance the image at either the lower or higher side of the thermal intensity range without altering the original image.

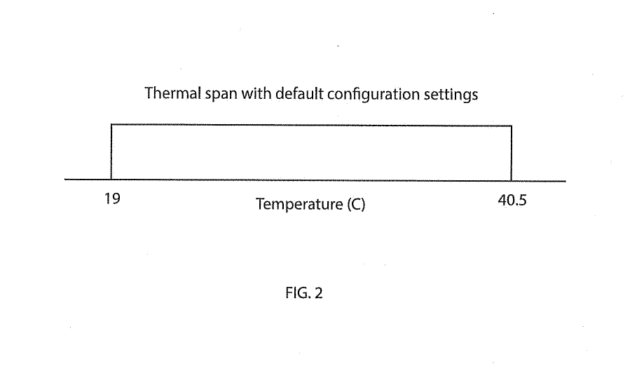

Referring to FIG. 2, the thermal imager could be configured to capture the thermal intensity variation information within a certain range of thermal intensity. Configuration settings were carefully chosen such that they capture all thermal intensity variations between 19° C. (66.2° F.) to 40.5° C. (104.9° F.), which covers most of the human body's physiologic thermal intensity range. When the thermal intensity of an area of interest gets close to 19° C. (66.2° F.), the pixel values in the grayscale thermal image appear darker and reach a low saturation point. When the thermal intensity drops below 19° C. (66.2° F.), the thermal image would still appear dark but would not get any darker as the low saturation point has already been reached. Similarly, as the thermal intensity of an area of interest starts increasing, the thermal image starts looking brighter. As the thermal intensity gets close to 40.5° C. (104.9° F.), the thermal image reaches the high saturation point and the pixel values in the grayscale image reach the maximum value. As the thermal intensity goes beyond 40.5° C. (104.9° F.), even though the thermal intensity of the area of interest is increasing, the thermal image would not appear any brighter as the high saturation point has been reached.

Even though the thermographic imager can pick up thermal intensities as low as 19° C. (66.2° F.) the grayscale thermal image for an area of interest at that thermal intensity would appear too dark. The human eye is not able to visualize the variation of the 254 pixel values included in the standardized grayscale. This might cause problems when thermographic images are taken on areas of the human body with decreased microcirculation, (i.e., the fingers, toes, etc.) or areas with cartilage (i.e., the tip of the nose, ear, etc.). These body locations are usually the coldest on the skin surface thermal intensity and would appear darker in the thermal images.

To solve this problem, a novel technique has been developed to increase or decrease the brightness of the pixel values by adding a positive or negative offset to the raw pixel values. The positive or negative offset allows an enhanced visualization of the black or white extremes in a grayscale image. The goal here is to visually enhance the image at either the lower or higher end of the thermal intensity range without altering the original image.

With default configuration settings and at a room thermal intensity of 22.11° C. (71.8° F.), the thermal intensity range picked up by the thermal imager was as illustrated in FIG. 2.

A low saturation grayscale value of 1 was reached at 19° C. (66.2° F.) and the high saturation grayscale value of 254 was reached at 40.5° C. (104.9° F.), giving a thermal span of 21.5 degrees. The maximum resolution is then 0.0846° C. with in the image.

Thermal Span (Thermal intensity range picked up by an imager)=(Thermal intensity at which the pixels reach the high saturation value)−(Thermal intensity at which the pixels reach the low saturation value): Formula:

$$Maximum resolution = \frac{( \begin{matrix}High saturation temperature - \\ low saturation temperature\end{matrix} )}{Resolution of the gray scale image}$$

For an 8-bit grayscale image the resolution is fixed at 254 parts.

Adding a positive offset (Example of Use):

When a positive offset +20 was added to all the pixels to make the image look brighter the imager reached the low saturation grayscale value of 21 at 19° C. (66.2° F.). Since a value of +20 is added to all the pixels, the grayscale value can only go as low as 21 instead of 1 as obtained with default settings. This lowest grayscale value was obtained at the same thermal intensity (19° C.) as the low saturation thermal intensity obtained with default settings. This indicates that adding an offset will only increase the pixel value making it look brighter so that small variations in the pixel values could be visually seen. This does not enable the thermal imager to pick up thermal intensities lower than what can be read with default settings.

With positive offset added, the image appears brighter and reaches the high saturation value at a thermal intensity lower than the high saturation thermal intensity obtained with default settings. The imager reached the high saturation thermal intensity at 39° C. (102.2° F.) instead of 40.5° C. (104.9° F.), as obtained with default settings.

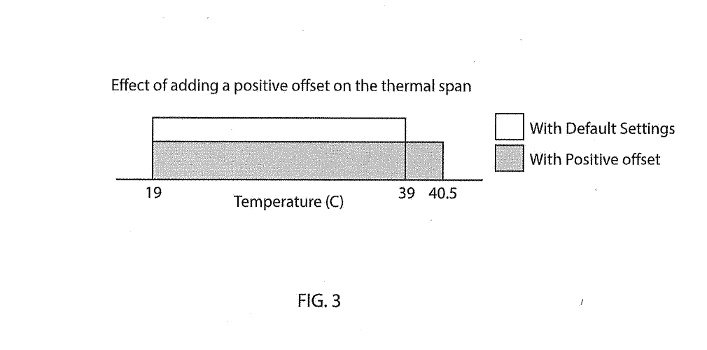

FIG. 3 shows the thermal intensity range that is detected when a positive offset is added to the default pixel value configuration setting.

The thermal span is reduced to 20 degrees instead of 21.5 degrees as obtained with default settings when a positive offset was added. The maximum resolution increased to 0.0855° C. which gives more definition to the pixels within the image.

Adding a negative offset (Example of Use):

Adding a negative offset to the raw signal coming from the imager makes the thermal image look darker, improving the visualization of the hot (bright) areas. When an offset of −20 was added to the original signal the pixel values reached the low saturation value of 1 at 20.5° C. (68.9° F.) instead of 19° C. (66.2° F.). Since the thermal images are saved as unsigned 8-bit grayscale images with pixel values ranging from 1-254, if the values fall outside this range they would be mapped to 1 or 254. So when a negative value is added, pixels with values less than 20 would become negative and were mapped back to 0 so that the pixel values always stay in the range of 1-254. Similarly on the high end the pixel values reached the highest saturation value of 234 at 40.5° C. (104.9° F.). With a negative offset added the highest the pixel values can go up to is 234 instead of 254. This high saturation occurred at the same thermal intensity as obtained with default settings.

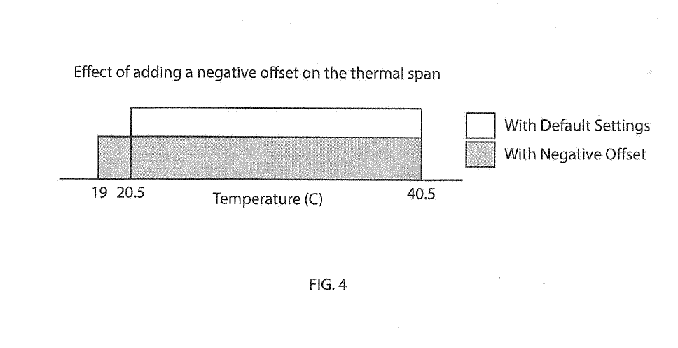

FIG. 4 shows the effect of adding a negative offset on the thermal intensity range that could be picked up by the thermal imager.

The thermal span is reduced to 19 degrees giving a maximum resolution of 0.0855° C. within the image.

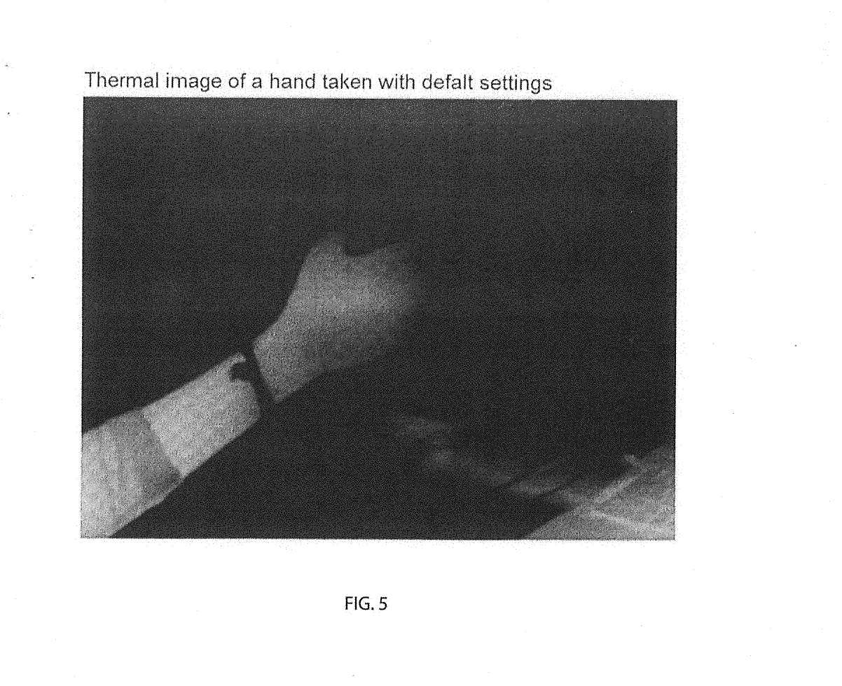

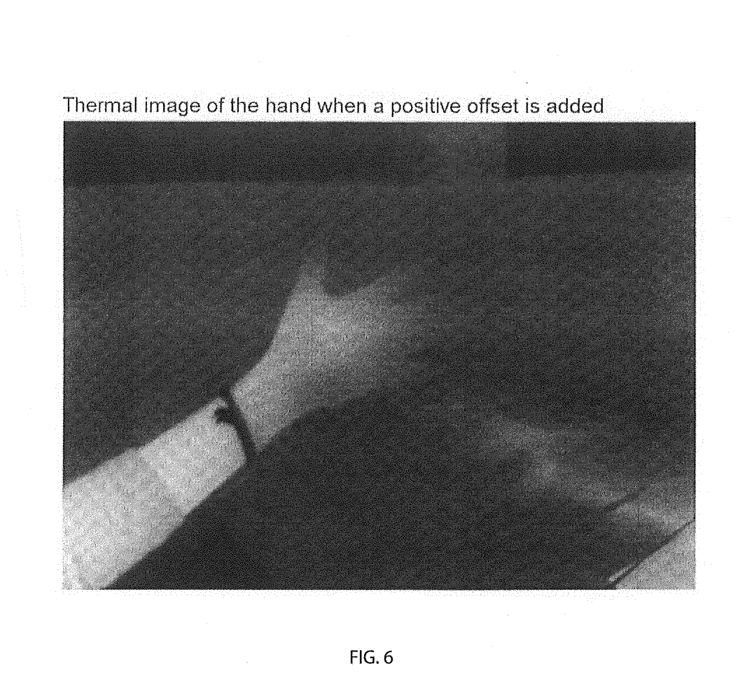

By choosing a suitable offset (positive or negative) value the visualization of an image is enhanced by increasing the resolution within the image. This concept has been implemented and proven by the researched thermal imaging. An offset of 20 was chosen as an example. This could change based on the requirements. FIG. 5 below shows a thermal image of a hand taken with default settings. FIG. 6 below shows an example of the effect on the thermal image when a positive offset is added to the pixel values at default settings to improve the visualization of the image.

B. Defining Pixel Intensity Variations in the Long-Wave Infrared Image:

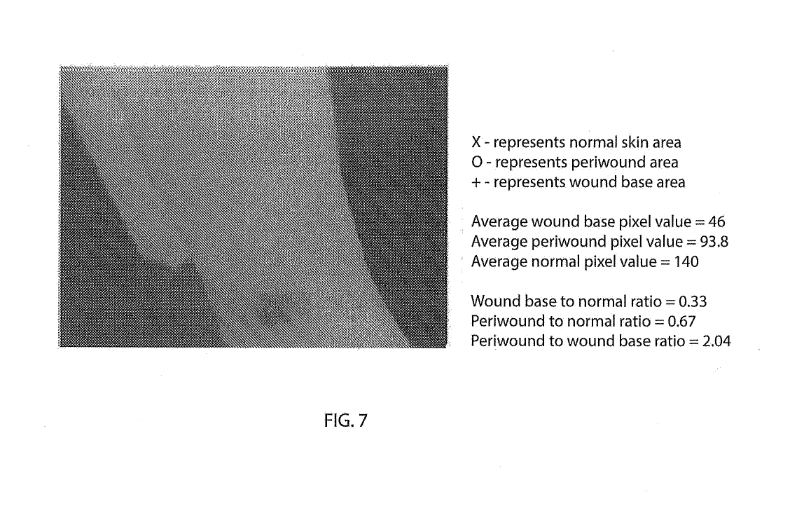

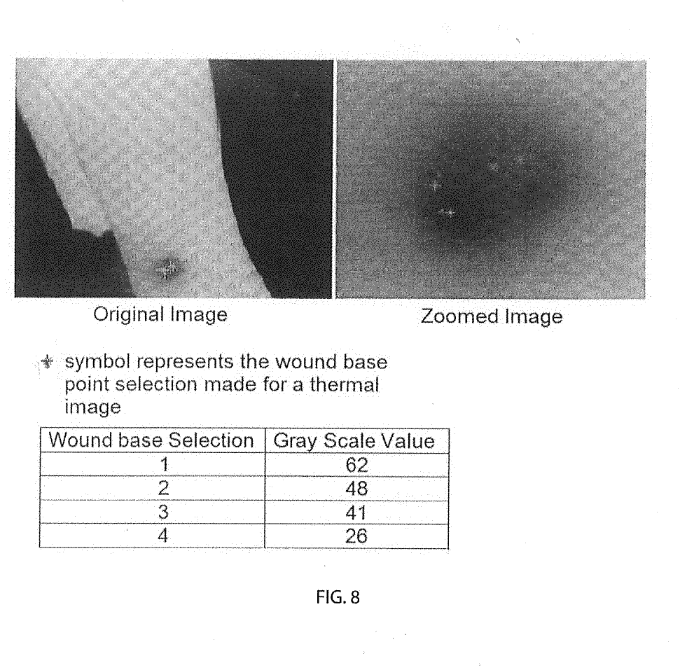

To assist the clinician to define the pixel intensity variations of the long-wave infrared image to see how thermal intensity is varying across the skin area of images taken, as well as previous thermal images of the same location, an inventive technique has been developed that measures the thermal intensity ratio. This gives the clinician the ability to look at the images captured with the thermal imager and choose pixel points in the image utilizing non-zoomed and zoomed presentations of the image that represent skin and subcutaneous tissue surrounding the area of interest. The clinician also has the ability to select the tissue in which an injury/wound exists as shown in FIGS. 7 and 8. The zoomed capability allows the clinician to be very precise in the selection of the pixels used to measure thermal intensity. The zoomed feature is particularly useful because of the complexity of various wound types. For example, the wound base and periwound can be disorganized (acute and chronic condition, etc.), organized (wound resurfacing or repairing, etc.), and/or infected (wound base infection with and without periwound cellulitis, etc.).

FIG. 7 shows a non-zoomed thermal image with unaffected and abnormal selections. The ‘X’ marks represent the unaffected skin, the asterisk symbol represents the wound base and the circle marks represent the periwound.

FIG. 8 shows an original and zoomed thermal image with abnormal selections. The table in the image shows selected points on the thermal image with their corresponding grayscale values.

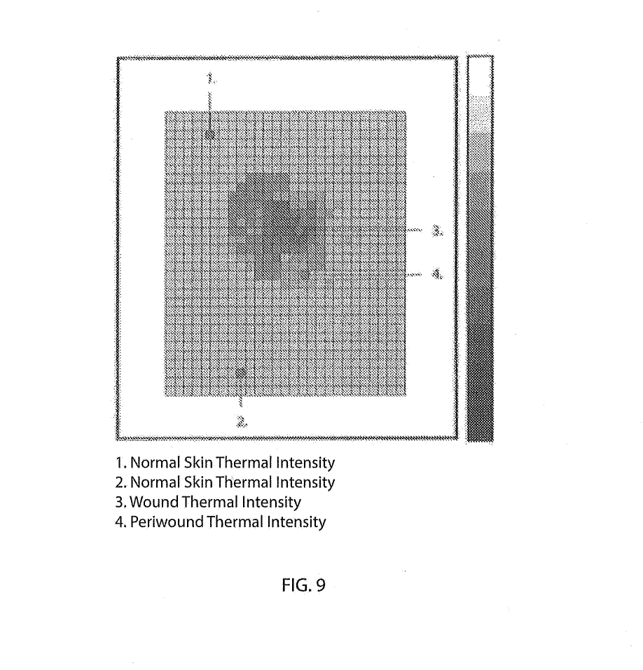

FIG. 9 shows a schematic representing pixel intensity recognition (zoomed).

Pixels with uniform gray color represent the unaffected skin and subcutaneous tissue. If the pixel value is too high, then it can be an indication of an infection developing in that area. The wound base is usually colder than the unaffected skin's thermal intensity and is represented with darker pixels on a thermal image. The pixel values for a periwound area are usually higher than the wound base pixel value and less than the pixel value associated with the unaffected tissue as their thermal intensity falls between the unaffected skin thermal intensity and the wound base thermal intensity.

The display of pixel value associated with each pixel selection made could help a clinician make a decision on whether an area of interest is present. This allows the following calculations to be performed:

Wound Base to Unaffected Ratio:

$$Wound base to unaffected ration = \frac{( \begin{matrix}Average of all the pixel values \\ from the wound base region\end{matrix} )}{( \begin{matrix}Average of all the pixel values \\ from the unaffected region\end{matrix} )}$$

Wound base regions are usually colder than the unaffected skin thermal intensity, causing the pixel values for the wound base regions to be lesser than the pixel values for the unaffected skin regions in an LIR image.

If the wound base to unaffected ratio is less than 1, it is an indication that the wound base is colder than the unaffected regional tissue. If the ratio is greater than 1, it is an indication that the wound base area is hotter than the regions selected as unaffected skin area. In summary, the closer the value gets to 1, the closer the wound base area is getting to unaffected skin.

Periwound to Unaffected Ratio:

$$Periwound to unaffected ratio = \frac{( \begin{matrix}Average of all the pixel values \\ from the periwound region\end{matrix} )}{( \begin{matrix}Average of all the pixel values \\ from the unaffected region\end{matrix} )}$$

If the periwound to unaffected ratio is less than 1, it indicates that the periwound is colder than the unaffected skin area. If the ratio is greater than 1, it is an indication that the periwound area is hotter than the regions selected as unaffected skin area. In summary, the closer the value gets to 1, the closer the periwound area is getting to unaffected skin.

Periwound to Wound Base Ratio:

$$Periwound to wound base ratio = \frac{( \begin{matrix}Average of all the pixel values \\ from the periwound region\end{matrix} )}{( \begin{matrix}Average of all the pixel values \\ from the wound base region\end{matrix} )}$$

The ratio greater than 1 indicates that the periwound region is hotter than the wound base region and the ratio less than 1 indicates that the wound base region is hotter than the periwound region. In summary, the closer the ratio gets to 1, the closer the wound base and periwound values get to each other.

By monitoring these ratios, the clinician could get a better idea on the status of the wound.

C. Maintaining Separation of the Imager from Target:

Long-wave infrared and visual images must be consistently taken at a predetermined distance, typically 18 inches. This capability allows measurements to be obtained by length×width, by linear measurement, and by encirclement of the area of interest and or wound. This information is considered to be the gold standard of the wound care industry in determining the progression or regression of abnormalities.

Thermal and visual cameras are used for capturing images of areas of interest, such as wounds in a real time fashion (i.e., bedside or outpatient clinic). Cameras are built so that they can communicate with computer via a USB connection and capture both visual and thermal images by clicking the trigger button on the camera.

All the images need to be captured at a certain distance from the body part and a standard distance of 18 inches between the camera and the body part was found in testing done to date to be an ideal distance. Several methods were used in order to measure this distance.

As a first attempt an antenna of length 18 inches was placed on the camera core that could be extended out. When the end of the antenna touched the body part the standard distance was known to have been attained, indicating that the camera is ready for capturing images. The adverse effects of using an antenna for measuring the distance were that the antenna would be touching the body part giving rise to possible risk of contamination, and also that the antenna comes into the field of view when the image is being captured causing problems with visualization.

To overcome these problems the antenna method was replaced with a more sophisticated method using ultrasonic sound waves. An ultrasonic transducer placed on the camera core would release ultrasonic sound waves for transmission in the desired path and when these waves hit the target, which would be the body part in our case, and ultra sonic sound waves would be reflected back from the target in the transmission path. The received ultrasonic sound waves can then be converted into an electrical signal that can be processed by a processor to provide distance information. The distance can be computed by using the time period from the middle time value of the received electrical signal to the middle time value of the transmitted signal. Whenever this distance equals the standard distance of 18 inches a reduced audible noise will be generated, indicating that the camera is ready to capture an image.

Even though the ultrasonic sound wave method has been proven to be successful and has been used in various applications to date for measuring the distances, it was never used in the medical field at bedside as a tool for capturing visual and thermal images.

Limitations of using the ultrasonic method included the complexity of wiring and the size of the apparatus used for measuring the distance and then displaying it so that the end user can see how far the camera is from the target. The other major limitation arose with the presence of an object in between the camera and the target. When there is an object in the path, part or all of the waves will be reflected back to the transmitter as an echo and can be detected through the receiver path. It is difficult to make sure that the received ultrasonic sound waves were actually reflected by the target and not by any other object in the path.

The ultrasonic measuring of the distance was replaced with the use of two Class I Laser LED lights. Two Class I A, or of less strength, lasers and/or LED modified lights are used in this method. These lasers emit narrow light beams as opposed to diffused light. They are placed on either side of the camera lens. When the distance between the camera and the target is less than 18 inches the lights coming from these lasers fall on the target as two spots separated by a distance and this distance will keep decreasing as the camera is moved toward from the target. When the distance between the camera and the target equal 18 inches the lights from these two light sources will coincide, indicating that the focus point has been achieved and that the camera is ready for capturing images. The distance between the two light beams starts increasing again when the distance between the camera and the target increases to the standard 18 inches.

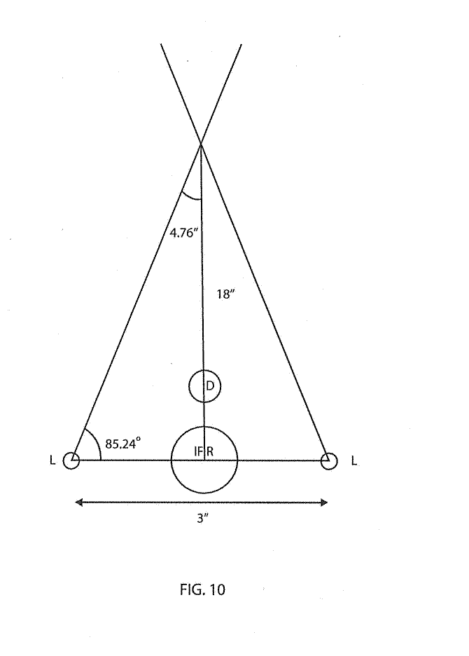

FIG. 10 explains the above embodiment in more detail, where IFR represents the long wave infrared microbolometer and D represents the visual digital camera, and L represents the laser lights.

Depending on how far the laser lights are going to be from the microbolometer and the distance between the microbolometer and the target the angles at which the lasers need to be inclined will change.

The digital camera ‘D’ is also going to be placed at around 1.5 inches away from the long-wave infrared microbolometer and in order to make both the digital and the long-wave infrared microbolometer to have the same focus point and field of view the digital camera needs to be inclined at an angle.

The experimental setup of FIG. 11 that was used in order to determine the angle of inclination is as shown.

FIG. 12 is a representation of an embodiment that uses 18 inches as the desired distance in a clinical setting. By changing the angles of the Class 1 Lasers this distance can be increased or decreased to meet other needs or requirements determined by the clinician.

D. A Consistent Technique to Obtain Wound Measurement Length and Width Linearly Using a Thermal Image:

To assist clinicians with maintaining accuracy and consistency when measuring a wound, a novel technique has also been developed to obtain consistent linear wound measurements (length and width) using a thermal image. It allows a clinician to follow a standard of care to determine the progression and regression of the wound by measuring length and width and area.

To be able to obtain the measurements of a wound from an image the number of pixels available per centimeter or per inch in that image needs to be known. When images are always taken from a standard distance the number of pixels per inch in that image always remain constant, and they change with the change in the separation distance between the imager and the target.

The imager has been designed such that the separation distance between the imager and the target is always maintained at 18 inches. Several techniques like using a measuring tape, using ultrasound and using Class 1 lasers have been tried and tested to date to maintain this standard distance. The final version of the imager makes use of two Class lasers mounted inside the imager at an angle such that the laser beams emitted from these two lasers always converge at 18 inches from the front of the camera.

For an image taken at a distance between the object being imaged and the imager that is exactly 18 inches there would be in the image approximately 40 pixels per inch. This distance can be changed, but at each distance the number of pixels needed to equal 1 cm or 1 inch must be measured and tested. The selected distance must be noted to maintain reproducibility. For the calculation of length and width of the wound, when a line is drawn across the area of interest by measuring the number of pixels covered across this line and using a conversion formula the measurement in pixels could be converted into inches or centimeters. For an image taken at 18 inches from the target, a line that is 40 pixels in length would be approximately 1 inch on the measuring scale and using the inch to centimeter conversion the length could then be converted into centimeters.

Algorithm for measuring length and width of an area of interest (in centimeters):

Draw a line across the image that represents the length or width of the area of interest that needs to be measured.

Note the x and y coordinates of the starting and ending points of this line.

If (x1,y1) represent the x and y coordinates of the starting point of the line and (x2,y2) represent the x and y coordinates of the end point of the line then the distance between these two points (length of the line in pixels) can be measured as:

$$Length ( or width ) in pixels = \sqrt{{( x 2 - x 1 )}^{2} + {( y 2 - y 1 )}^{2}}$$

$$Length ( or width in inches = \frac{Length in pixels}{40} \\ Length ( or width ) in centimeters = \frac{Length in pixels}{15.7480}$$

As per Minimum Data Set (MDS) Version 3.0, it is recommended that the length of a wound is always measured as the longest length drawn from head to toe and width is measured as the widest width drawn side to side perpendicular to the length. The x or y coordinates of the end point of the line representing the length or the width line could be adjusted to make sure the lines are exactly vertical or horizontal which would in turn make them perpendicular to each other.

Using the length and the width measurements (length×width) area could be calculated.

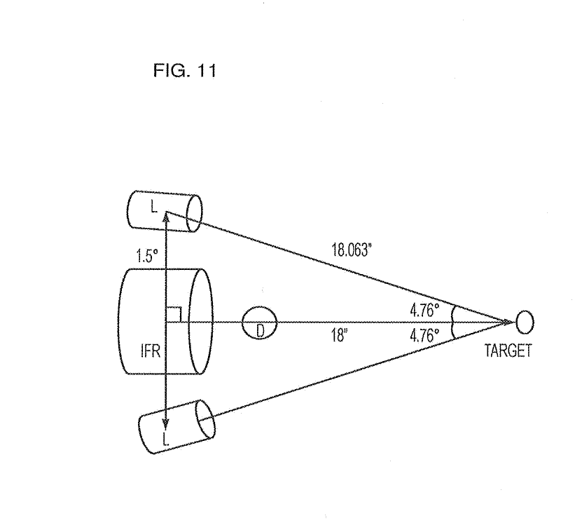

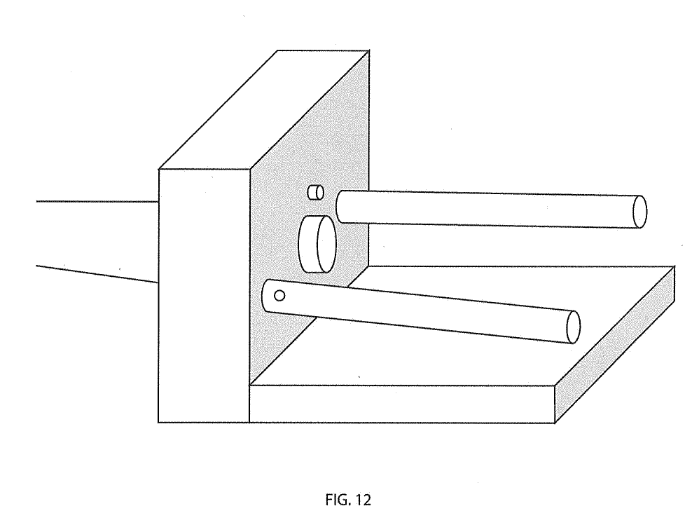

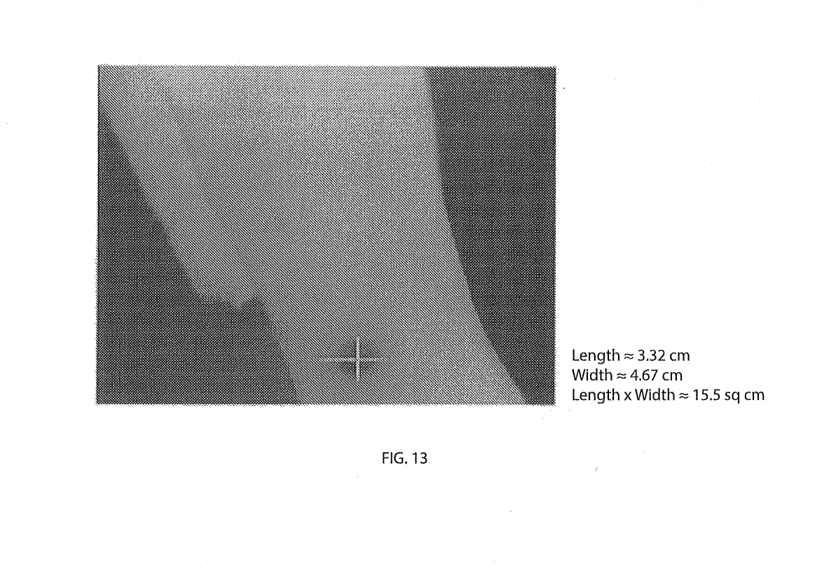

By monitoring the thermal images taken on day to day basis, and by measuring the length and the width for the area of interest each day, the status of the wound could be monitored to see whether there has been a progression or regression in the status. FIG. 13 shows the length and width measurement in centimeters obtained for an image with an area of interest on a heel.

E. Highlighting the Wound Base, Periwound and Unaffected Regions to Measure and Calculate the Square Areas Thereof by Using the Number of Pixels Highlighted:

A novel technique has been developed that gives the clinician the ability to highlight a wound base, periwound or unaffected regions and to measure the area in square centimeters. This will assist the clinician in looking at the overall status of the wound, and evaluating its progression or regression.

The total number of pixels enclosed within the highlighted area could be used for calculating the area of the region selected.

A test target of size 1.5 inch×1.5 inch was used. With the imager at 18 inches from the test target, images were captured.

The area of test target=1.5 inch×1.5 inch=2.25 square inches or 3.81 cm×3.81 cm=14.5161 square cm.

For an image taken at 18 inches from the target there would be approximately 40 pixels per inch. So there would be approximately 60 pixels in 1.5 inches.

The area of the test target obtained from the image=60 pixels×60 pixels=3600 pixels. A total of 3600 pixels were enclosed inside the area of the test target. So 3600 pixels↔14.5161 square cm

For an unknown area of interest, if “Y” is the number of pixels enclosed inside that area then the surface area in square centimeters for that region would be equal to:

$$Area in square centimeters for the highlighted region = \frac{( Y \times 14.5161 )}{3600}$$

For the region highlighted as the wound base, its area in square centimeters and the average of all the pixel values falling inside the highlighted region are calculated and displayed in the picture as shown in FIG. 14.

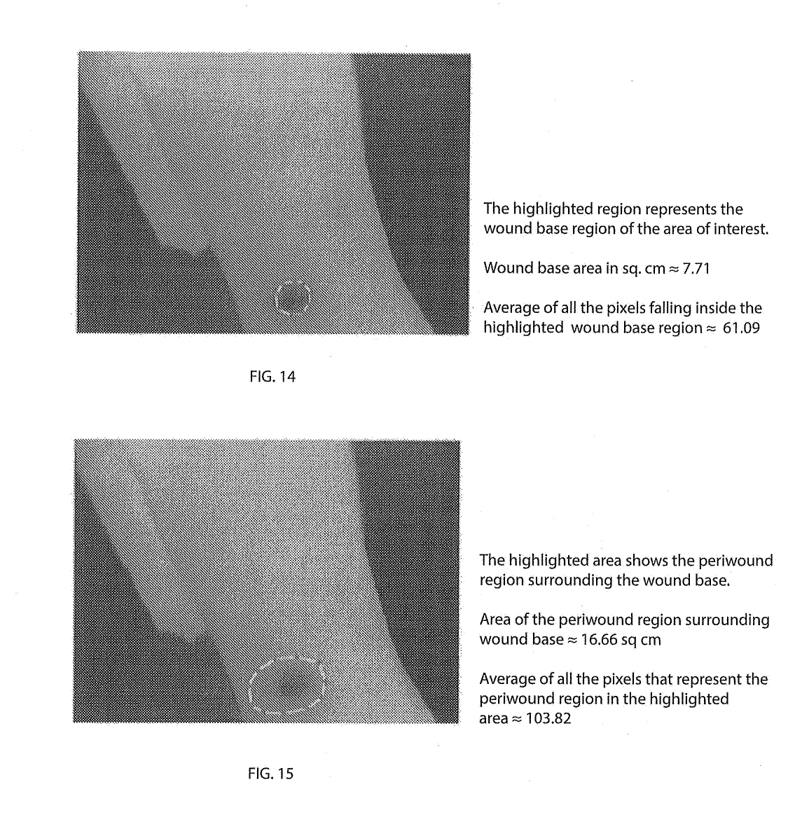

Periwound area represents the area surrounding the wound base. By highlighting the area that includes the wound base and the periwound area surrounding it as shown in FIG. 15, and by counting the number of pixels enclosed in that region, the area of the highlighted region could be calculated in square centimeters. The periwound area could then be obtained by subtracting the wound base area from the area that includes both the periwound and the wound base areas.

By including the unaffected skin and subcutaneous tissue surrounding the wound in the highlighted area of interest, the unaffected area could be calculated in square centimeters. The unaffected area could then be obtained by subtracting the wound base and periwound area from the region selected that includes unaffected, periwound and the wound base areas.

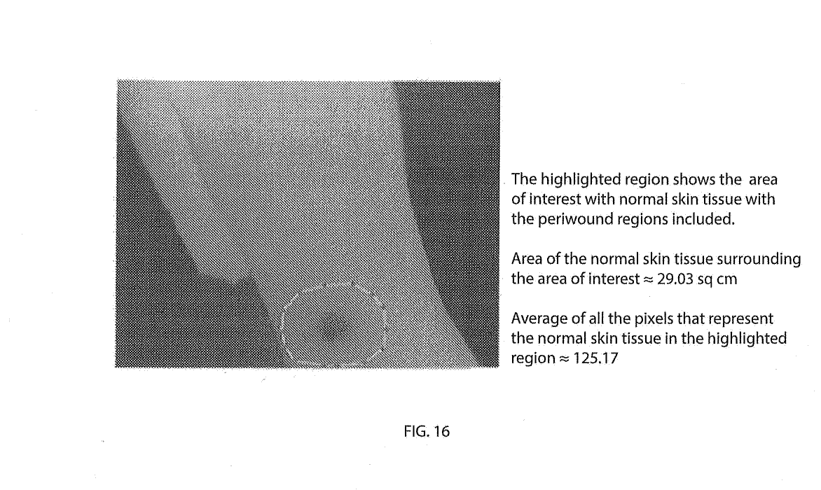

FIG. 16 below shows the calculations displaying the highlighted unaffected area and the various calculations obtained from the highlighted regions.

F. Obtaining the Average Pixel Value and the Plus/Minus Variance by Encircling the Area of Interest/Wound:

By utilizing the novel techniques above, not only can the area be calculated, but simultaneously the average pixel value of each area can be calculated. This will allow the clinician to evaluate the status of the area of interest or wound not only in micro (focused technique, above) but also in the macro using the technique described below. The combination of these two assessments will give a better overall understanding of the areas of interest where the abnormality or wound has been identified. From this average data the ratio concept discussed above can also be used to evaluate the macro (overall) look at an area of interest or wound, specifically if the wound is becoming organized, i.e., is it improving, becoming infected, or regressing (getting worse). See Table 1 below.

TABLE 1
Summary of Results Obtained From the
Highlighted Normal, Periwound, and Wound Base Regions

| | Normal | Periwound | Wound Base |
|---|---|---|---|
| Area in sq. cm | 29.03 | 16.66 | 7.71 |
| Average pixel value | 125.17 | 103.82 | 61.09 |
| Minimum and | [Various range] | [Various range] | [Various range] |
| maximum pixel | | | |
| values | | | |

Some of the other measurements that could be done to keep track of the status of an area of interest include calculating the average, minimum and maximum of all the pixel values falling inside the highlighted area.

$$Average pixel value = \frac{ \begin{matrix}Sum of pixels values for all the pixels \\ that fall inside the highlighted area of interest\end{matrix}}{Total number of pixels falling inside the highlighted area}$$

For a highlighted area of interest a histogram can be generated to provide graphical representation of distribution of pixel values within that area.

Algorithm for Generating Histograms:

Step 1: Highlight the area of interest for which a histogram needs to be generated.

Step 2: Determine the total number of bins/buckets into which the data needs to be divided into. There is no best number of bins, and different bin sizes can reveal different features of the data.

Step 3: Bin size can be calculated as

$$Bin size = \frac{Maximum value - Minimum value}{Total number of bins}$$

For a thermal image the pixel values always range between 0 and 255.

Step 4: Create an empty array of size equal to the total number of bins.

Step 5: Check to see if a pixel falls inside the highlighted area of interest and if it does note the pixel value.

Step 6: The bin number into which this pixel value falls under can be calculated using the formula:

$$Bin number = \frac{Pixel value - Minimum value}{Bin size}$$

Step 7: Increment the value of the array at the index [Bin number—1], since arrays are zero based, by one.

Repeat Steps 5-7 for all the pixels in an image.

After checking all the pixels in an image, plot the array to generate a histogram.

Clinical significance of histograms:

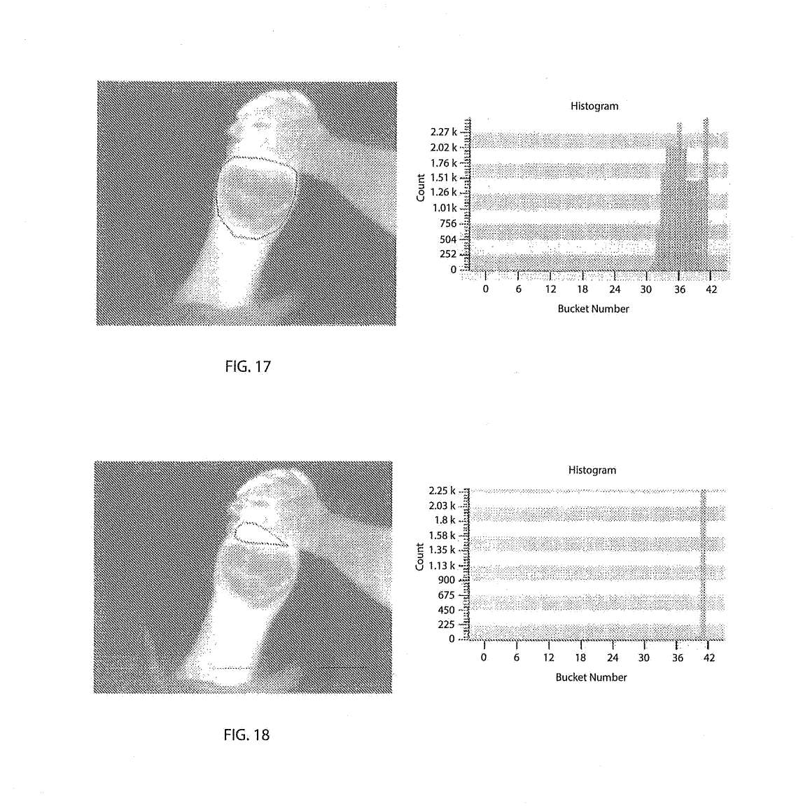

Distribution of pixel values as projected by the histograms for a highlighted area of interest provides more in depth information about the signature of a wound. If the histogram plot is more spread out it indicates there is a large variation in the pixel values and hence temperatures within the highlighted area as shown in FIG. 17. As the plot starts getting more and more narrow it is an indication that all the pixels inside the highlighted portion are getting close to each other and the temperature inside the highlighted portion is starting to get saturated towards a single temperature value. If the saturation occurs at a higher pixel value then it is an indication that all the pixels inside the highlighted portion are getting very hot compared to the selected normal reference point. Similarly if the saturation occurs at a very low pixel value then all the pixels inside the highlighted area are getting very cold. FIG. 17 shows some sample histograms generated for an image with a highlighted area of interest.

G. Creating Profile Lines in and Through an Area of Interest/Wound and Comparing with Profile Lines Trough Reference Areas:

A novel feature has been developed to assist a trained clinician to better track a wound by utilizing the ability to plot profile lines through the wound. These plots show the variation in the pixel values across the wound. Since the thermal intensity is directly related to the grayscale pixel values in an image, these plots can be used to monitor how the thermal intensity is varying across an area of interest or wound. This allows the clinician to dissect the wound in precise fashion, so the pathophysiologic status of the wound can be assessed and quantified.

Profile lines can be plotted by simply drawing a line across the area of interest. FIG. 18 below shows an example of the profile line generated by drawing a line across the wound present on the heel. As seen in the plot there is a huge drop in the pixel value/thermal intensity across the wound base region and the value starts increasing as the line is moving away from the wound base and entering areas with unaffected skin tissue.

As the wound starts healing the difference between the pixel value for the unaffected tissue and the pixel value from the wound base starts decreasing and hence the drop seen in the graph starts decreasing indicating that the wound is healing and is starting to get close to the unaffected skin tissue.

If the drop in the pixel values starts increasing, when plots are generated for images taken on timely basis then it is an indication that the wound is deteriorating and that the clinician needs to turn to strategies to facilitate wound healing.

Algorithm for Generating the Profile Lines:

Draw a line across the area of interest for which the profile lines need to be plotted.

Record the X and Y locations of the starting and end points of the profile line. Let (x1,y1) represent the coordinates of the starting point and (x2,y2) represent the coordinates of the end point.

deltaX=absolute value of (x2−x1); deltaY=absolute value of (y2−y1)

length of the line=L\sqrt{(x2−x1)²+(y2−y1)²}

x_increment=deltaX/L

y_increment=deltaY/L

Round off L to the nearest integer and then increment by 1; L=L+1 Create a new array to hold the pixel values that fall across the profile line. Let us call this array ‘Pixel values’, wherein Pixel_values(1)=pixel value of the image at the location x1, y1. Add the x_increment and y_increment to the original x1 and y1 respectively and use these as new values for x1 and y1. So x1=round (xi+x_increment) y1=round (y1+y_increment).

Create a new counter variable, let us call it ‘i’.
Set i=1;
While (i<L) and (x1,y1 fall within the size of the image),
Pixel_values (i+1)=pixel value of the image at the location x1,y1;
x1=round (x1+x_increment);
y1=round (y1+y_increment);
i=i+1;
End

The array ‘Pixel_values’ should contain values of all the pixels that represent the profile line.

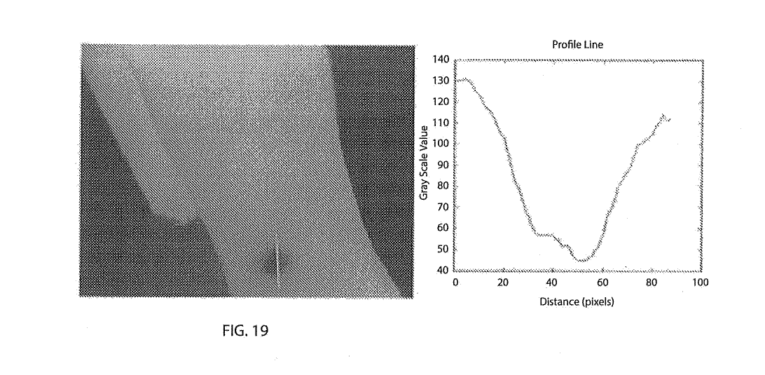

Plotting the values in the array ‘Pixel_values’ gives the plot for the profile line drawn across the area of interest (as shown in FIG. 19).

Images taken using a thermal imaging camera can be analyzed and tracked to monitor the status of wounds.

Profile lines provide a tool for monitoring variations in pixel values and hence the temperatures across the abnormal areas of interest. These variations can be compared against the pixel value representing unaffected region for that patient by selecting a region on the image that represents unaffected skin.

Comparing the profile line with the reference line representing the unaffected skin for that patient:

Comparing the pixel values of the pixels falling across the profile line with the reference pixel value that represents unaffected skin for that patient gives a measure of how close or far away the profile line pixel values are from the selected reference line.

For selecting unaffected regions a circle can be drawn on the image that comprises of only the unaffected pixels and does not include any abnormalities or the background. Once a circle has been drawn representing unaffected skin for the patient, average of all the pixels falling inside the circle can be calculated as follows:

$$Average Normal pixel value = \frac{ \begin{matrix}Sum of all the pixels that fall \\ inside the circle representing Normal\end{matrix}}{Total number of pixels inside the circle}$$

To determine whether a pixel falls inside a circle of radius ‘r’ calculate the distance between the center of the circle and the coordinates of the pixel point using the formula

Distance=\sqrt{(x2−x1)²+(y2−y1)²}

where (x1,y1) represent the X and Y coordinates of the center of the circle and (x2,y2) represent the X and Y coordinates of the pixel.

If the distance is less than the radius of the circle then that pixel falls inside the circle representing unaffected skin area.

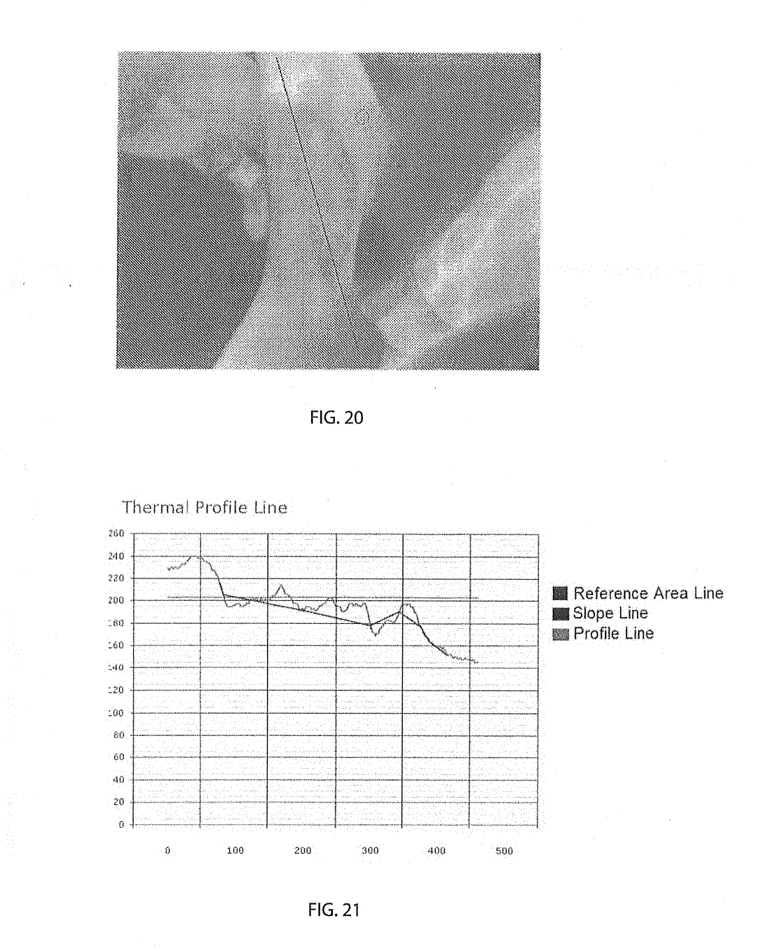

Once the average normal pixel value has been calculated this value can be plotted on the chart along with the profile line as shown in the FIGS. 20 and 21.

By comparing the profile line with the normal line the status of the area of interest can be tracked. As the profile line gets closer to the reference line it indicates that the area of interest is improving and is getting closer to the normal skin characteristics.

The portions below the reference line represent the segments of the profile line where the pixel values are lower (colder) than the selected normal reference point. Similarly the points falling above the reference line represent the portion of the profile that is hotter than the selected normal reference.

Once a normal reference point has been chosen and a profile line has been drawn several parameters can be calculated to compare the profile line signature with the reference line signature. By tracking how these values change on day to day basis the status of the wound could be tracked.

Some of the factors that could be calculated to compare the profile line with the reference line include area above and below the reference line, maximum rise and drop, average rise and drop from the reference line etc.

The area calculations also give a measure of the portion of the profile line that falls above or below the normal reference line. The area that falls above the reference line indicates the regions that have a pixel value higher that the reference point and hence are at a higher temperature. The area below the reference line shows the portion of the profile line that has temperatures lower than the selected reference.

The areas can be calculated using the Trapezoidal Rule of calculating area under the curve.

Calculating Area Above and Below the Reference Line:

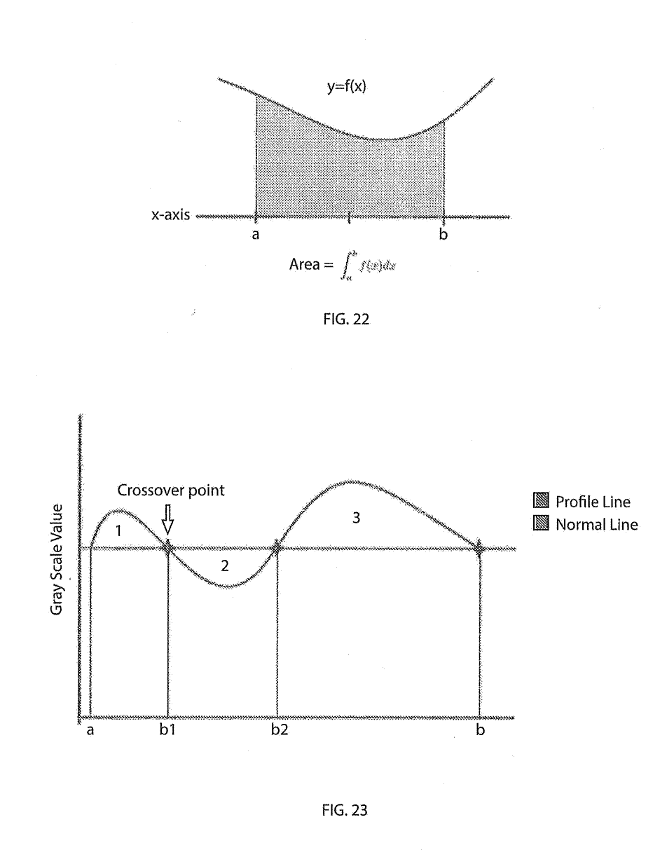

The area between the graph of y=f(x) and the x-axis is given by the definite integral in FIG. 22 (Reference: http://www.mathwords.com/alarea_under_a_curve.htm) This formula gives a positive result for a graph above the x-axis, and a negative result for a graph below the x-axis.

Note: If the graph of y=f(x) is partly above and partly below the x-axis, the formula given below generates the net area. That is, the area above the axis minus the area below the axis.

The Trapezoidal Rule (also known as the Trapezoid Rule or Trapezium Rule) is an approximate technique for calculating the definite integral as follows:

$${\int }_{a}^{b} f ( x ) dx ≅ \frac{Δ x}{2} * ( f ( x 0 ) + f ( xn ) + f ( x 2 ) + … + f ( x ( n - 1 ) ) ) )$$

where

$$Δ x = \frac{( b - a )}{n} ,$$

x0=a,x1=a+Δx,x2=a+2Δx . . . xn=a+nΔx=b and ‘n’ is the number of equal length subintervals into which the region [a, b] is divided.

To calculate area relative to the Normal line, instead of x-axis, pixel values relative to the selected normal need to be calculated which equal the actual pixel value minus the selected normal value.

The relative pixel value being positive indicates that the point falls above the normal line, and being negative indicates that it falls below the normal line. Whenever the relative pixel value across the curve goes from positive to negative or vice versa it is an indication that there has been a crossing of the normal line. The algorithm for computing the area above and below the normal line can be summarized as follows:

1. Calculate relative pixel values;

2. Find out where the crossover points occur;

3. Split the curve into positive and negative regions;

4. Calculate area for each region separately using the Trapezoidal Rule; and

5. Combine all positive areas to obtain area above normal line and all the negative areas to obtain the area below the normal line.

FIG. 23 shows a plot of a sample profile line and a normal line. As shown in the Figure, the sample profile lines crosses the normal line at three points dividing the curve into three regions. Regions 1 and 3 fall above the normal line and have positive relative pixel values, whereas the region 2 falls below the normal line and has negative relative pixel values.

To calculate the area above and below the normal for the sample plot the area for the three regions need to be calculated individually using the Trapezoidal Rule:

$$Area for the region 1 = {\int }_{a}^{b 1} f 1 ( x ) ≅ \frac{Δ x}{2} * ( f 1 ( a ) + f 1 ( b 1 ) + 2 * ( f 1 ( x 1 ) + f 1 ( x 2 ) + … + f 1 ( x ( n - 1 ) ) ) )$$

where f1 (x) defines the curve in region 1,

$$Δ x = \frac{( b 1 - a )}{n} ,$$

x1=a+Δx,x2=a+2Δx . . . xn=a+nΔx=b1, and ‘n’ is the number of equal length subintervals into which the region [a,b1] is divided.

$$Area for the region 2 = {\int }_{b 1}^{b 1} f 2 ( x ) ≅ \frac{Δ x}{2} * ( f 2 ( b 1 ) + f 2 ( b 1 ) + f 2 ( b 2 ) + 2 * ( f 2 ( x 1 ) + f 2 ( x 2 ) + … + f 2 ( x ( n - 1 ) ) ) )$$

where f2(x) defines the curve in region 2,

$$Δ x = \frac{( b 2 - b 1 )}{n} ,$$

x1=b1+Δx,x2=b1+2Δ . . . xn=b1+nΔx=b2, and ‘n’ is the number of equal length subintervals into which the region [b1, b2] is divided. The area for this region would be negative indicating that it falls below the normal line.

The area above the Normal line can be obtained by adding areas under regions 1 and 3. Thus:

Area Above the Normal Line=∫ₐ^{b1}f1(x)+∫_{b2}^{b}f3(x)

The area below the Normal line (i.e., the Area under the region 2)=∫_{b1}^{b2} f2(x)

By counting exactly how many number of pixels fall above or below the reference line the percentage of profile line that falls above or below the profile line can be calculated as follows:

$$Percentage of profile line that falls above the reference line = \frac{( Number of pixels that fall above the reference line ) * 100}{Total number of pixels across the profile line}$$

$$Percentage of profile line that falls below the reference line = \frac{( Number of pixels that fall below the reference line ) * 100}{Total number of pixels across the profile line}$$

$$Percentage of profile line that falls along the reference line = \frac{( Number of pixels that fall on the reference line ) * 100}{Total number of pixels across the profile line}$$

Maximum rise above the reference line gives the maximum positive difference in the pixel values between the profile line and the reference line. A rise in this value indicates that the temperature for some of the pixels along the profile line is getting much hotter than the reference value and decrease in this value indicates that the maximum difference between the profile line pixel values and the reference line pixel values is decreasing and that the profile line is getting closer to the reference line.

Similarly, maximum drop below the reference line can be calculated as the maximum negative difference in the pixel values between the profile line and the reference line. An increase in the maximum drop indicates that the pixels on the profile line are colder than the average reference pixel value.

Average rise and average drop can also be used as factors for comparing the profile lines with the reference line. Formulae for calculating average rise and average drop are as follows:

$$Average rise above the reference line = \frac{Sum of all the pixels that fall above the reference line}{Total number of pixels that fall above the reference line}$$

$$Average fall below the reference line = \frac{Sum of all the pixels that fall below the reference line}{Total number of pixels that fall below the reference line}$$

Slopes: Calculating slopes for the profile lines gives information about how often the temperature varies along the profile line. A slope line can be drawn on the profile line every time there has been a significant change in the pixel value (temperature). A positive slope indicates an increase in temperature and a negative slope indicates a drop in the temperature. The steepness of the slope lines indicates the amount of variation in temperatures. The steeper the lines the larger the variation is temperatures and the more irregular the profile line is.

An algorithm for calculating slopes and generating slope lines across the profile line can be summarized as follows:

Select a suitable value for slope variance, a value which indicates how much of a difference in pixel values between two points on the profile line is considered as a signification change.

Consider the starting point of the profile line as the starting point of the first slope line. Starting from this point and by moving along the profile line calculate the difference between the current pixel value and the pixel value at the starting location. If the difference is greater than or equal to the slope variance, the point at which the difference exceeds the slope variance becomes the end point for the slope line.

Draw a line on the profile line joining these two points.

Slope for this line can be calculated as follows:

If (x1,y1) represents the x and y coordinates of the starting point and (x2,y2) represent the coordinates of the end point of the slope line then the slope for this line can be calculated as:

$$Slope = \frac{( y 2 - y 1 )}{( x 2 - x 1 )}$$

Save this slope value in an array.

Make the end point of the first slope line as the start point for the next slope line to be generated and repeat step 2 to determine the new end point.

Once the start and end points of the slope lines is established plot the slope line on to the profile line and then calculate and save the slope values.

Repeat the process until the end of profile line is reached.

FIG. 21 shows a slope line plotted on to the profile line with a slope variance of 12.

These are some of the factors that can be calculated from the profile line and reference line plots that help define the signature of the area of interest.

All the activity done by the clinician on the images can be recorded and saved in a database. The information can be retrieved on a later date to see which regions were selected as area of interest on that particular day, and to see what changes have occurred and how the results have changed with time. This novel approach will enable a trained clinician to better evaluate the area of interest/wound of the skin and subcutaneous tissue in a standardized and reproducible format.

The benefits related to using this advancement in long-wave infrared thermal imaging spans improvement in potential care, fulfilling regulatory requirements and fiduciary responsibility by reproducible and standardized documentation and cost savings secondary to the ability of clinicians to formulate appropriate individualized care plans for prevention, early intervention and treatment of abnormalities of the skin and subcutaneous tissue.

H. Using the Profile Line Plot to Interpret Wounds:

Once a profile line is drawn on the image across the area of interest a profile line plot can be generated using the algorithm outlined above. The plot can then be used to determine where on the profile line a drop or rise in the pixel value (temperature) occurs. The profile line plot can be made interactive so that when the user clicks on the plot the corresponding location on the image can be highlighted and hence making it easier to interpret. The algorithm for implementing this can be briefly summarized as follows:

1. Generate an interactive plot for profile line using tools like Telerik.

2. Create a chart item click event for the plot so that when the user clicks on the profile line plot the x and y values of the click point are recorded.

3. The X axis value at the click point (saved as ‘index’) shows how far away the point falls from the start point of the profile line. The Y value gives the actual pixel value at the point.

4. To locate this point on the profile line drawn on the image, the actual X and Y coordinates on the image need to be determined. The X and Y coordinates of the click point can be obtained as follows:

5. Calculate the length of the profile line using the start and end coordinates of the profile line.

6. If (X1,Y1) represents the coordinates of the starting point of the profile line on the image and (X2,Y2) represent the end point then the length can be calculated as

7. length of the line=L=\sqrt{(x2−x1)²+(y2−y1)²}

8. deltaX=absolute value of (X2−X1); deltaY=absolute value of (Y2−Y1)

9. x_increment=deltaX/L; y_increment=deltaY/L

10. if (x_increment>0 && y_increment<0) {index=L−index:}

11. The X and Y coordinates of the point that represents the click point can then be obtained as X=X1+(index*x_increment); Y=Y1+(index*y_increment);

12. Draw a string on the image at the X and Y coordinates from the previous step to indicate the click point.

Similar techniques can be used to determine where a point on the image falls on the profile line. The algorithm for doing this can be outlined as follows:

1. Add a Mouse down click event for the image.

2. Note the X and Y coordinates of the point where the user clicked on the image.

3. Check whether this point falls on the profile line

4. If the point falls on the profile line calculate the distance between the start point of the profile line and the point where the user clicked.

5. This distance indicates how far the point falls on the plot from the start point of the graph.

6. Draw on the graph to indicate this point.

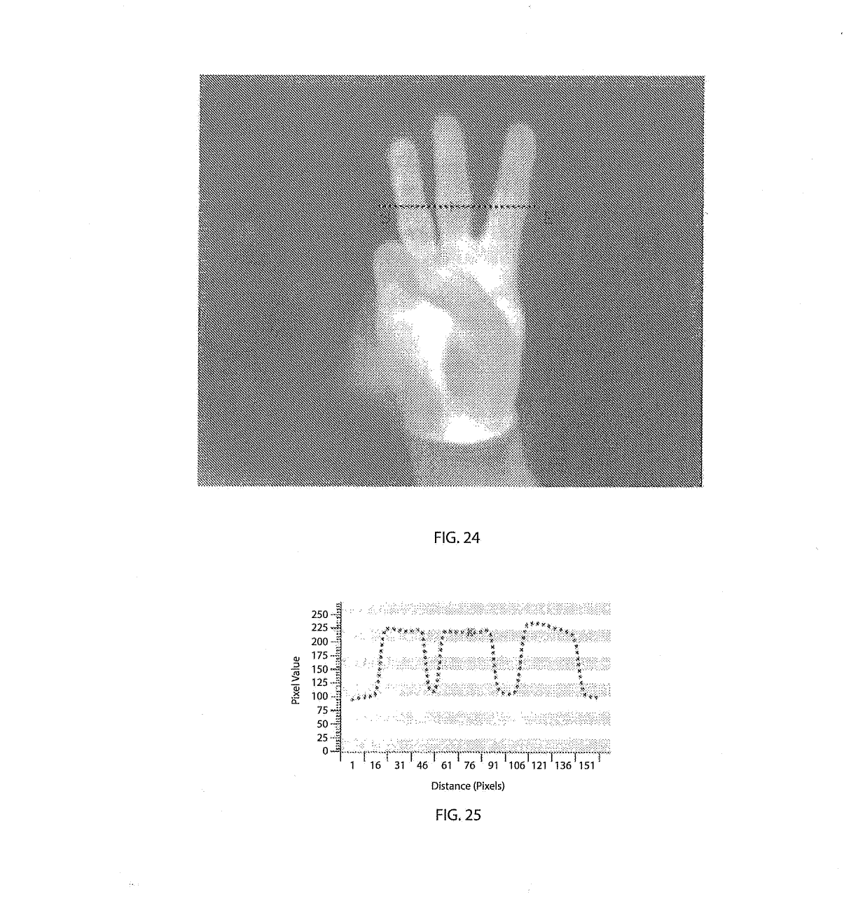

FIG. 24 shows a profile line drawn on the image of a hand and FIG. 25 shows the profile line plot. The X mark on the graph and the image indicates the user's selected point.

I. A Study Regarding Accuracy and Reproducibility of a Wound Shape Measuring and Monitoring System:

The current clinically accepted standard for measuring wound healing is for a clinician to use a hand ruler to measure the wound bed length (head to toe) and width (side to side) perpendicular (90-degree angle) to one another. The area can then be calculated by multiplying length×width (L×W) for area in centimeters squared.

The ruler L×W measurement method is quick and noninvasive; however, the area calculated is often inaccurate, as wounds are rarely squares or rectangles. The deviation from the true area is obviously dependent on multiple factors, including head direction, wound bed size, and shape. Despite the widespread use of this method for wound measurement, studies have documented that wound area calculated via this method overestimates the area by 44% or even greater. The ruler method does yield reproducible results from measurement to measurement, albeit highly inaccurate measurements.

Measurement of wounds from digital photographs and tracing of wound edges directly on acetate are other methods available to determine wound area. Although they are more complex to do at the bedside, they have been shown to provide more accurate measurements of wound length, width, and area than manual ruler measurements.

Thermal (infrared) and visual wound imaging can be accomplished using the WoundVision Scout. The Scout is a medical imaging device designed to photograph and measure area of a wound. The Scout is a clinical tool in the wound care arena to monitor change in wound size over time. It is a handheld, easy-to-use device that is portable and can collect digital visual (color) and digital long-wave thermal (infrared) images of external wounds and surrounding unaffected skin surfaces. Thermal and visual images are captured simultaneously for side-by-side comparisons. Once the images are captured, the digital visual image can be used to calculate wound L×W area and measure wound perimeter.

The thermal digital images captured by the Scout have a resolution of 640×480 pixels with 8-bit thermal intensity data per pixel. The resultant thermal images are displayed as a grayscale where thermal intensity values range from 1 (black) to 254 (white). The Scout thermal sensor can detect variations in temperature between 22° C. and 42° C., ideal for evaluating thermal changes of the body surface. WoundVision Scout does not provide absolute temperature values; rather, it provides relative thermal intensity values for evaluation. This is due to factors such as variation in ambient room temperature, environmental effects, and variation in clinically normal and abnormal body temperature among individuals.

The Scout has two, red class 1 range-finding lasers. The two projected red “dots” are designed to overlap to become a single point when the device is 18 inches from the body surface. This provides the software measurements with a known reference distance for comparability of images. The imager can be moved closer or farther away from the body surface, provided a reference object of known size (e.g., a disposable paper ruler) is placed within the field of view of the visual image. The Scout is a noncontact, noninvasive, non-radiating device that is considered safe to use for taking thermal and visual images for both the patient and the user.

The Scout ImageReview application runs on a standard personal or laptop computer. Only images that have been previously acquired and archived in the Scout database may be analyzed with this software. The visual function allows measurement and documentation of the wound size in the software by taking the longest length (head-to-toe) by width (90-degree angle to length) to calculate area (in centimeters squared) of the wound bed. This visual function emulates the criterion standard of current practice of measuring the external wound bed by the ruler method and allows the user to trace the external wound bed edges and measure both area (in centimeters squared) and perimeter (in centimeters) of the wound. The thermal function measures relative thermal intensity of a specified area. (The tracing and thermal image properties are not included in this study.) The user may overlay the visually traced external wound bed perimeter onto the thermal image to measure the relative thermal intensity variation data of the wound bed.

Completed ImageReview sessions included the original unaltered images, any tracing or graphical overlays, calculated results, and the basic identifying patient information, all of which is stored in a database to allow for later review. The completed work session information can also be exported to a portable document format report.

Precise and accurate wound measurement is critical to objectively evaluate healing, to determine if progress is being made toward closure, and to determine if the treatments are appropriate or need to be modified. The objectives of this study were to (1) demonstrate whether the Scout L×W methodology was equivalent to the criterion-standard ruler technique for measuring area in centimeters squared; (2) compare the accuracy of three methods of area measurements (ruler L×W, the Scout L×W, and the Scout tracing method); and (3) compare intrarater and interrater reliability of measurements taken of known sized shapes.

A prospective design was used to conduct this study. The study included both a shape measurement and shape imaging portion. A single shape assessment (measurement) and imaging session was executed for each preassigned head direction for all 19 shapes.

Development of Shapes:

Nineteen different shapes were cut from aluminum using a computer numerical control machine to achieve exact predetermined size for comparison to test for accuracy. Each of the 19 shapes was placed into its own shape matching Styrofoam frame. The Styrofoam frames were spray painted black to reduce glare. One side of the Styrofoam frame had three different straight lines drawn 45 degrees apart and marked 1, 2, and 3, respectively, to indicate the three different head directions of the shape to be measured and imaged for a total of 57 unique figures. Head directions were placed and marked identically on each frame of Styrofoam, and participants were directed to always point the indicated head direction straight up. A circle was drawn on each Styrofoam front, close to the shape as a target for the lasers.

Images were excluded if any of the following existed: (1) laser dots obscuring shape edge in digital visual images; (2) blurred digital image; (3) image could not be confirmed to be at 18 inches (lasers powered off or not overlapping); (4) images were not taken at approximately 90 degrees perpendicular to the shape; (5) digital visual images were too bright (shape edges cannot be seen); (6) digital visual images were too dark (shape edges cannot be seen); and/or (7) digital image did not upload (archive) properly.

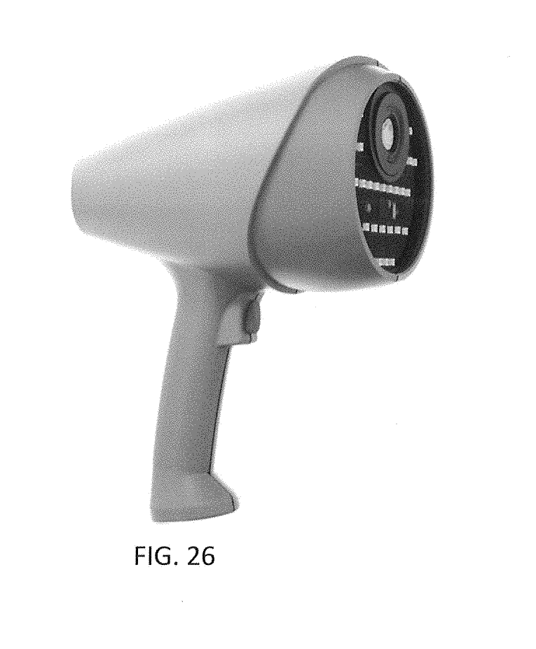

Participants had to be familiar with wound care and the Wound Monitoring and Measurement System. The three nurse participants received the same training on the shape tracing techniques of the visual images. A PowerPoint slide series was provided to familiarize them with study protocol and ImageReview software prior to commencing the study. Sample visual images of shapes were included in the PowerPoint illustrating how to measure the L×W using a ruler, the Scout ImageReview L×W, trace the outer edge of the shape, and how to take an image. A question-and-answer session and practice time were provided before the first data were collected to allow the participants to become familiar with the data collection process and Scout ImageReview software. This session focused on how to trace the outer edge of the shape. Participants were instructed to identify and then trace the outer edge of the black metal shape serving as the “wound.” The study monitor was available to answer questions on the training PowerPoint and prepared practice images. The participants were paid a small stipend for their time. FIG. 26 illustrates the device.

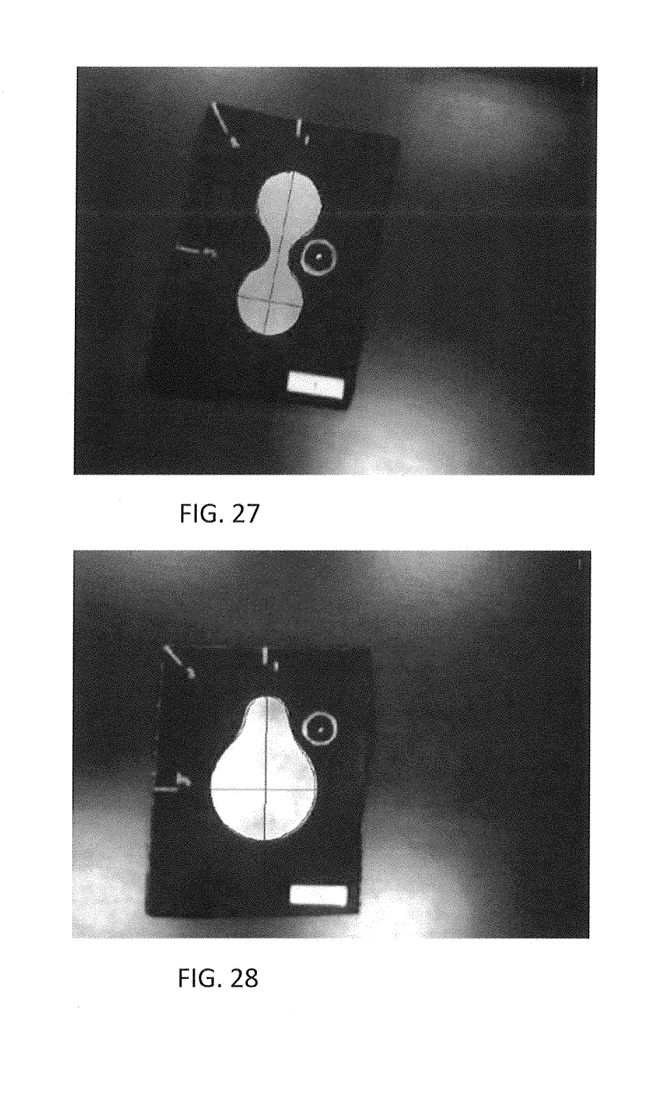

Three nurse clinician participants completed the study; all performed measurements on the same set of 19 shapes previously described (FIGS. 27 and 28). A randomization sequence was utilized for the evaluation order of the study images. Each shape was measured six times: twice with a ruler, twice with the WoundVision Scout L×W measure, and twice with the Scout tracing method. The shape assessment case report form (CRF) required the participant to perform manual measurements according to the indicated head directions. It also required the operation of the Scout device to obtain images and use the Scout ImageReview v1.1 software to measure L×W and the Scout tracing method. The shape assessment CRF was used as an original document where the data were first recorded.

Shape Assessment:

The shapes were placed flat on a table to emulate a supine patient. The handheld Scout device has no attachments. Participants used the current practice criterion standard of a ruler to measure the longest length (head to toe) first followed by the widest width perpendicular (90-degree angle) to the length. “Blinded” rulers without marked increments were used so that a number could not be recalled as a means of minimizing carryover memory and resultant bias.

After the “blinded” measurements were completed, the respective lengths and widths marked on the “blinded” rulers were measured in centimeters. The “blinded” rulers were labeled by the participant with the shape identification and head direction, and length 1, width 1, length 2, and width 2. The first set of “blinded” measurements was completed before taking the second set of “blinded” measurements.

Shape Imaging:

Participants, using the Wound Monitoring and Measurement System, obtained two visual images for each of the preassigned head directions of the 19 shapes. Starting with the first visual image, the participants used the Scout to measure the shape's L×W, emulating the criterion-standard technique used in the shape assessment portion. The participants then used the Scout to trace the outer edge of the shape. Using the second visual image, the participants then repeated the steps of measuring the shape's L×W and tracing the outer edge. This sequence of events was followed for each of the 57 unique figures.

Specific Instructions for Measurements Using the Scout Imager:

Both the imager control pad and indicated head direction were to face the same direction. The imager has a double-action trigger mechanism. A half-pull of the trigger turns on the light ring and the two class 1 lasers prior to capturing an image. With a full pull of the trigger, both a visual and thermal image is captured. When the two laser beams intersect, the thermal camera is 18 inches from the skin/shape and at the optimal distance for imaging. The lasers were not to be pointed directly on the shape as this impaired optimal image visualization. Rather, the participants pointed and imaged the laser beams in the circle provided next to the shape. A correct image occurred when the lasers intersected in the circle outside the shape (image angle is approximately 90 degrees perpendicular to the shape).

On the images, the participants were able to measure the longest length (head to toe), followed by the longest width (side to side) and perpendicular to length. This was done by using the cursor and making a single left click in the center of the shape. A red-tipped compass appeared. The red tip of the compass was pointed toward the indicated head direction by “left clicking” (holding the left click down) on the red-tipped pointer and dialing it until the red tip was pointed parallel to the axis of the head direction. The “left click” was then released, and the compass moved to the upper-right-hand corner as a reference. Dialing the compass parallel to the head direction allowed the longest length to be drawn and saved only at +10 or −10 degrees to the compass head direction. The longest length (head to toe) was then drawn by clicking on the edge of the shape closest to the head direction and then clicking again at the farthest edge opposite the head and releasing. The widest width could be drawn and saved only at +10 or −10 degrees perpendicular to the longest length. To draw the widest width (perpendicular to the longest length), the participants clicked on one side of the shape's edge and then clicked again at the opposite side and released. When the L×W lines were green, they were angled within the necessary+10 or −10 degrees, and if not, the L×W lines were red and could not be saved. Once the participants finished the L×W of each shape, the participants clicked on the “Trace Area of Interest” button, and then, they traced the perimeter of the shape's visual image. By “left clicking” the mouse, the pen-shaped cursor was used to anchor the trace of the perimeter edge of the shape. The participants were to “left click” more frequently to anchor the tracings for edges that were not straight. A double “left click” of the mouse joined the end of the tracing to the beginning. The participants viewed the image's L×W and tracing before uploading and saving their work.

A Case Report Form (“CRF”) was developed for use in this study. The CRF and the Scout database were the two source documents for this study. A paper data collection form was utilized by nurse clinician participants to capture shape assessment/measurement data. The study monitor validated the completed CRFs and transfer of data into electronic format. Clinician participants signed the CRF after completing their data collection to confirm completion of the image evaluation steps as directed.

Intrarater reliability for each nurse clinician participant was assessed via the repeatability coefficient at an a of 0.05% (95% confidence interval). The Coefficient of Individual Agreement (CIA) was used to test for equivalence of methods via the mean-squared deviation disagreement function. The coefficients of individual agreement, which are based on the ratio of the intrareader and inter-reader disagreement, provide a general approach for evaluating agreement between two fixed methods of measurements or human observers. Bland-Altman plots were constructed to assess level of agreement between methods (objective 1). The CIA was used to test for agreement between participants' tracings (interrater reliability). Participant measurements were compared with known objects measurements via a t test for (1) L×W manual measurement and the actual known shape areas; (2) L×W manual measurements and the L×W software measurements from the visual images; and (3) the visual image area measurements by tracing and the known shape areas. The squared SD indicates the variance or range in the data.

Mean Area Measurements. The mean of the true area is the actual mean of all shapes “true” area. The mean areas demonstrate that both the reference standard ruler L×W and the Scout L×W have a tendency to overestimate the true area of the shape. The Scout tracing measurements are the closest to the true area (Table 2). Four of the shapes are circles that should have the same area when rotated.

TABLE 2
MEAN AREAS (cm²) AS DETERMINED BY THE RULER AND
SCOUT METHODOLOGIES, FOR THE COMPLETE DATA SET

| Methodology | n | Mean | SE | 95% CI |
|---|---|---|---|---|
| True area | 19^{a} | 9.65 | 4.59 | 10.02-29.29 |
| Ruler L × W area | 342^{b} | 26.42 | 1.40 | 23.67-29.17 |
| Scout L × W area | 342 | 27.10 | 1.47 | 24.21-29.98 |
| Scout tracing | 342 | 20.70 | 1.11 | 18.52-22.88 |

^{a}Each shape regardless of orientation has only one (1) true area.
^{b}Total equals three (3) orientations of 19 shapes, all three (3) participants, two (2) measurements each (3 × 19 × 3 × 2 = 342).
Abbreviations:
CI, confidence interval;
L × W, length × width.

To determine if inclusion of those circles influenced the results, the analyses were completed with and without the rotated measures for these circles. Table 3 includes the results utilizing only one orientation of the four circles.

TABLE 3
MEAN AREAS (cm²) AS DETERMINED BY THE RULER
AND SCOUT METHODOLOGIES, EXCLUDING THE
CIRCLES AT TWO (2) ORIENTATIONS

| Methodology | n | Mean | SE | 95% CI |
|---|---|---|---|---|
| Ruler L × W area | 294^{a} | 28.43 | 1.55 | 25.39-31.48 |
| Scout L × W area | 294 | 29.20 | 1.63 | 26.00-32.41 |
| Scout tracing | 294 | 22.29 | 1.23 | 19.87-24.71 |

^{a}Each shape other than circles (n = 15), all three (3) shape orientations, all three (3) participants two (2) measurements each (3 × 15 × 3 × 2 = 270); plus four (4) circles, one (1) orientation, three (3) participants, two (2) measurements each (n = 24), for a total of 294 measurements.
Abbreviations:
CI, confidence interval;
L × W, length × width.

When comparing the data in Tables 2 and 3, the results are similar. The mean area measurements and the variability are slightly greater when the duplicate circle measurements are removed. However, this observed difference was about the same across the three methods. Therefore, inclusion of the circles at each orientation should not impact the comparison between the methodologies.

Mean Area by User. The mean area as determined by the individual nurse participants has a similar pattern across the participants (Table 4). The overestimation of true area by the L×W area measurement methodologies was observed consistently. The ruler and Scout L×W area measurements are similar, whereas the Scout tracing area is closer to the true area regardless of participants making the assessment.

TABLE 4
MEAN AREAS (cm²) BY USER

| | | Nurse 1 Mean | Nurse 2 Mean | Nurse 3 Mean |
|---|---|---|---|---|
| Methodology | n | (95% CI) | (95% CI) | (95% CI) |
| Ruler L × W | 114^{a} | 26.78 | 25.76 | 26.72 |
| | | (21.95-31.61) | (20.99-30.53) | (21.90-31.54) |
| Scout L × W | 114 | 27.21 | 27.13 | 26.96 |
| | | (22.10-32.32) | (22.10-32.15) | (21.94-31.97) |
| Scout tracing | 114 | 20.47 | 20.76 | 20.87 |
| | | (16.65-24.30) | (16.95-24.56) | (17.05-24.70) |

^{a}Each shape and each orientation measured twice for each nurse participant separately. Measurements were made against the “true area” of 19.65.
Abbreviations:
CI, confidence interval;
L × W, length × width.-

Accuracy. A comparison was made between the “true mean” and the mean as measured by the ruler L×W, the Scout L×W, and perimeter tracing. This was done to test which method yielded the best estimate of absolute or true area. Two measures were used: the absolute difference from the true area and the percent difference from the true area (Tables 5 and 6). Absolute difference is a measure of the absolute variation or error in the measurements, whereas percent difference from the true area multiplies the actual error by 100% as another way of expressing error.

TABLE 5
ABSOLUTE DIFFERENCE FROM TRUE AREA

| | | Absolute Difference From True Area (cm²) | | |
|---|---|---|---|---|
| Methodoloy | n | Mean | SE | 95% CI |
| Ruler L × W | 342^{a} | 6.85 | 0.42 | 6.02-7.68 |
| Scout L × W | 342 | 7.98 | 0.49 | 7.00-8.96 |
| Scout tracing | 342 | 1.12 | 0.73 | 0.97-1.26 |

^{a}Each shape regardless of orientation has only one (1) true area.
Total equals three (3) orientations of 19 shapes, all three (3) participants, two (2) measurements each (3 × 19 × 3 × 2 = 342)L × W.
Abbreviations:
CI, confidence interval;
L × W, length × width.

TABLE 6
PERCENT DIFFERENCE FROM TRUE AREA

| | | Percent Difference From True Area | | |
|---|---|---|---|---|
| Methodology | n | Mean | SE | 95% CI |
| Ruler L × W | 342^{a} | 39.97 | 1.64 | 36.74-43.20 |
| Scout L × W | 342 | 37.17 | 1.53 | 34.16-40.19 |
| Scout tracing | 342 | 4.36 | 0.32 | 3.73-4.99 |

^{a}Each shape regardless of orientation has only one (1) true area.
Total equals three (3) orientations of 19 shapes, all three (3) participants, two (2) measurements each (3 × 19 × 3 × 2 = 342).
Abbreviations:
CI, confidence interval;
L × W, length × width.

The mean percent difference from the true area clearly shows that the Scout tracing methodology yields an area estimate closer to the true area (4.36%). The percent differences for the ruler L×W and Scout L×W area are similar (39.97% and 37.17%, respectively) (Table 6). The results are similar to those seen when the circles at two orientations are excluded (Table 7).

TABLE 7
MEAN PERCENT DIFFERENCE FROM TRUE AREA AS
DETERMINED BY THE RULER AND SCOUT METHODOLOGIES,
EXCLUDING CIRCLES AT TWO (2) ORIENTATIONS

| | | Percent Difference From True Area | | |
|---|---|---|---|---|
| Methodology | n | Mean | SE | 95% CI |
| Ruler L × W | 294^{a} | 39.83 | 1.80 | 36.28-43.38 |
| Scout L × W | 294 | 37.98 | 1.66 | 34.72-41.24 |
| Scout tracing | 294 | 4.14 | 0.33 | 3.48-4.80 |

^{a}Each shape other than circles (n = 15), all three (3) shape orientations, all three (3) participants, two (2) measurements each (3 × 15 × 3 × 2 = 270); plus four (4) circles, one (1) orientation, three (3) participants, two (2) measurements each (n = 24), for a total of 294 measurements.
Abbreviations:
CI, confidence interval;
L × W, length × width.

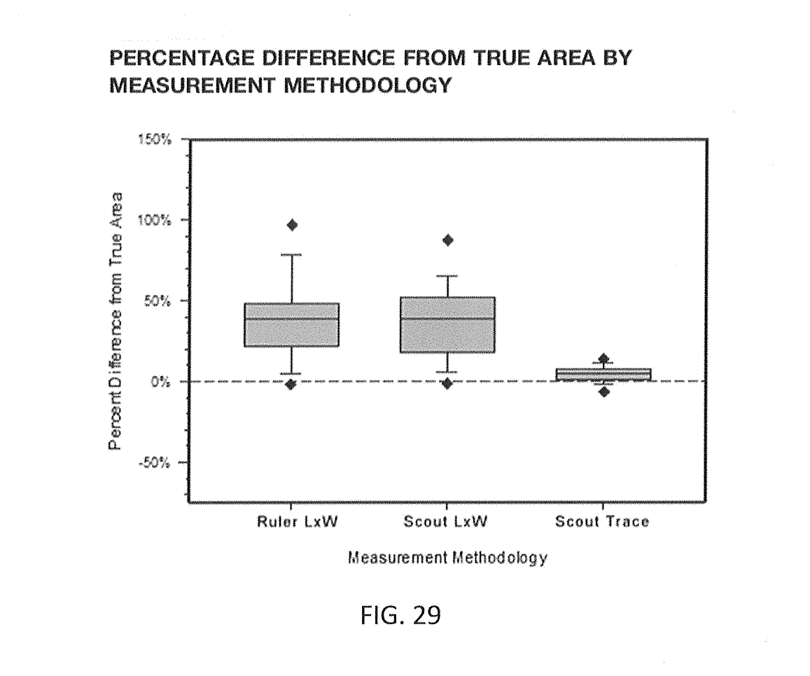

The Scout tracing method was the most accurate measure of area when compared with the true area, with an estimate on average approximately 4% to 5% different than the true area (true area 19.65) (FIG. 29). Both the ruler and the Scout L×W measurements tend to overestimate the true area by a significant margin (37% to 40%) (FIG. 29).

Comparison of Ruler and Scout Area Measurements:

When the CIA methodology was used to compare the Scout L×W methodology with the reference standard ruler L×W methodology, the percent difference in area was utilized. The methodologies were determined to be equivalent with the 95% CI, including 1.

Comparison of Overall Intrareader (Nurse Participant) Measurements. Table 8 illustrates the percent difference between the first and second measurements (intrareader reliability) for each nurse participant for each method (intrareader). For example, the mean percent difference in measurements 1 and 2 for the ruler method for participant 1 was 0.25%. The percent difference in measurements for the Scout L×W methodology for participant 1, although higher than the ruler methodology, was only 1.44%.

TABLE 8
THE PERCENT DIFFERENCE BETWEEN THE
TWO (2) MEASUREMENTS MADE BY EACH
NURSE PARTICIPANT FOR EACH METHOD

| | Ruler L × W Area | | Scout L × W Area | | Scout Tracing | |
|---|---|---|---|---|---|---|
| | Mean | | Mean | | Mean | |
| | (SE) | 95% CI | (SE) | 95% CI | (SE) | 95% CI |
| Participant 1 | 0.25 | (−1.70 to | −1.44 | (−4.02 to | −0.15 | (−1.65 to |
| | (0.97) | 2.20) | (1.29) | 1.15) | (0.75) | 1.35) |
| Participant 2 | −0.63 | (−1.76 to | −2.98 | (−5.36 to | −1.12 | (−2.25 to |
| | (0.56) | 0.49) | (1.19) | 0.61) | (0.56) | 0.007) |
| Participant 3 | −0.30 | (−1.98 to | 0.24 | (−2.22 to | −0.19 | (−1.29 to |
| | (0.84) | 1.39) | (1.23) | 2.69) | (0.56) | 0.90) |
| Overall | −0.23 | (−1.15 to | −1.39 | (−2.81 to | −0.49 | (−1.20 to |
| percent | (0.47) | 0.69) | (0.72) | 0.02) | (0.36) | 0.22) |
| difference | | | | | | |

Abbreviations: CI, confidence interval. L × W, length × width.

The 95% CIs almost all include 0, thus demonstrating good consistency from nurse participant to nurse participant for all three methods. All of the Est_Psi_N values are greater than 1.

For all three methods (ruler L×W, Scout L×W, and Scout perimeter trace), the intrareader measurements are acceptable. The CIA Psi_Nvalues are greater than 0.8. Thus, all three methods reliably measure area from measurement to measurement.

Comparison of Nurse Participants. Some nurse participants showed agreement for each methodology, but none of the methodologies were performed consistently by all participants. All three methods gave reproducible results on repeat measurements. For all methods, the variability from participant to participant was greater than that on repeat measures made by the same nurse participant (indicating if one or more nurse readers were consistently accurate or inaccurate in their readings). The CIA (Est_Psi_R) is 0.77, demonstrating that the reference standard ruler/caliper surface measurement method is equivalent to the Scout L×W surface measurement method (FIG. 29).

Study Conclusions:

Precise and accurate wound measurement is critical to objectively evaluate healing. Previously, precise and accurate wound measurement was not always the easiest to accomplish. Based on the results of this study, there is a new, accurate, and clinically feasible method for wound measurement.

It is recognized that the aluminum shapes used in this study to simulate wounds have a sharp border, leaving less discrepancy in identifying wound borders as compared with actual wounds. This methodology was used to first establish accuracy of the WoundVision device for wound measurement. Because actual wound area is difficult at best to determine, using the device on shapes with known areas was necessary. The subsequent study is testing the device on a variety of actual wounds in clinical settings and will be reported in the future.

Based on the established CIs in this study (CIA), the Scout L×W methodology was shown to be equivalent to the criterion-standard ruler L×W area measurement (Psi_R=0.77; 95% CI, 0.528 to 1.016), yet both methods tended to overestimate true area of the shapes. For all three methods, the variability from nurse participant to nurse participant (interrater reliability) was greater than that on repeat measures by the same nurse participant (intrarater reliability). None of the three methodologies were consistently performed by all nurse clinician participants.

All three measurement methods yielded reproducible results on repeat measurements. All three nurse participant measurements were equivalent based on the CIA results (Table 6). Utilization of the Scout provides both an equivalent measure of L×W area to the criterion standard and the ability to more accurately measure true area of the shape (wound) by utilizing the tracing method. The deviation from the true area is obviously dependent on multiple factors, including head direction, wound bed size, and shape.

Wounds are rarely, if ever, a square or rectangle. Using the ruler L×W measurement method results in an area measurement often greatly exceeding that of actual or true area. In reality, this method provides measurements that are consistently variable and wholly inaccurate. In practice, clinicians using the ruler L×W measurement tend to subjectively measure the longest length and then the widest width, not necessarily perpendicular to one another. When the head-to-toe length and wide-to-side width ruler method, perpendicular to one another, is used, there is greater chance of some consistency between measurers. Wounds rarely heal symmetrically; therefore, a tracing of the perimeter of the wound would provide the most accurate estimation/measurement of true area. In addition, having one consistent method that is consistently used from clinician to clinician provides the greatest chance of comparability of measurements over time.

In practice, it can be challenging to achieve patient adherence to plan of care. Having a numerical and/or pictorial printout of the decrease (or increase) in wound area over time to share with a patient has been found to serve as a motivator for adherence.

Future research using the WoundVision technology on actual wounds would add to the scientific body of knowledge on precise, accurate, and clinically feasible wound measurement techniques. For use of the technology of the thermal camera, further research on capturing what is actually occurring in and around the wound could provide valuable pathophysiological data for diagnosing wound etiology and assessing the effectiveness of treatment interventions.

J. A Study Regarding Comparison of Standardized Clinical Evaluation of Wounds Using Ruler Length by Width and Scout Length by Width Measure and Scout Perimeter Trace:

Currently, the accepted standard for wound measurement is to use a hand ruler to measure wound size. Although there are several variations on the ruler method, a common practice is outlined by the National Pressure Ulcer Advisory Panel (NPUAP) on its website and in the NPUAP Pressure Ulcer Scale for Healing (PUSH) Tool version 3.0. The PUSH Tool is designed to document data on a complete pressure ulcer assessment, which is then tabulated for a total score. Clinicians can use the PUSH Tool to document healing or wound deterioration over time. The website and PUSH Tool instruct clinicians to measure the longest length of the wound head to toe and then the longest width of the wound, taking the width measurements perpendicular to the length measurement. This technique resulted in the least overestimation of wound area discussed in the study described above in Section I.

The manual ruler method is quick and noninvasive, but the area measurements are almost always inaccurate as the length×width (L×W) technique assumes a square or rectangular wound shape. The deviation from the true area is dependent on multiple factors, including wound bed size and shape, which are easily distorted by body position. Studies have shown wound area calculations using the L×W ruler method can overestimate area by 44%, especially for wounds with irregular edges. Although highly inaccurate, the ruler method yields fairly reproducible results from measurement to measurement. Therefore, measurement of wound size over time provides a fairly reliable measure of change in wound status.

Other devices on the market for measuring wound size involve tracing wound edges directly on acetate and using digital photographs. Measuring wounds from digital photographs, although more complex to use bedside, has been shown to provide more accurate wound measurements than the ruler method.

This study measured 40 patient wounds to demonstrate the performance of an instrument new to the market, called the Scout device, on actual wounds in the intended clinical population.

The Food and Drug Administration-approved Scout device (WoundVision, LLC, Indianapolis, Ind.), previously known as the Wound Measurement and Monitoring System, has two (2) main components: the Scout ImageCapture and the Scout ImageReview software. The ImageCapture is a combination digital camera and long-wave infrared camera. The digital camera is indicated for the use of capturing visual images of a part of the body or two body surfaces. The long-wave infrared camera is indicated for the use of capturing thermal images. The ImageReview software allows for measurement of the diameter, surface area, and perimeter of wound images and the thermal intensity variation data of a part of the body or two body surfaces.

Intended for qualified healthcare personnel who are trained in its use, the Scout is a noncontact, noninvasive, non-radiating device. The Scout is considered safe to use (for both patient and user) for capturing both visual and thermal images.

This study was institutional review board-approved and was conducted in compliance with the protocol, good clinical practices, and all applicable regulatory requirements. All investigational staff members were trained on the protocol and the proper use of the Scout ImageReview. There was no anticipated benefit to the study subjects who participated in this study. However, the images collected may lead to the improved care in the future.

A prospective design was used to retrospectively analyze collected images of actual patient wounds from 40 patient subjects from both an inpatient and an outpatient setting.

The study objectives were to (1) compare the L×W ruler method and wound area calculation to the Scout L×W method and the perimeter trace method of visual wound area measurement and (2) to establish within and between reader agreement of the Scout L×W, Scout trace area, and Scout trace perimeter (measurements of trace area and perimeter).

Following institutional review board approval, 40 actual patient wounds were imaged at an inpatient and an outpatient clinical site in Indiana to represent feasibility of the Scout in both inpatient and outpatient clinical settings. The 40 patient wounds were of various etiologies, representing those commonly seen on an inpatient and outpatient basis (e.g., venous, neuropathic, arterial, and pressure ulcers). This study used both experts in clinical wound care (n=3) and nonexpert readers (n=2). The five study readers included a physician, a registered nurse, a licensed practical nurse, and two readers familiar with the device but not experts in clinical wound assessment. The expert readers were clinicians trained in wound care and in the appropriate use of the Scout system. Previous study data of the researchers, as well as other peer-reviewed literature, suggest that variation in qualitative wound characteristics (wound edge) exists not only between readers of different experience levels and training, but also between readers of similar specialized training and experience.

Multiple clinicians measuring wounds in a clinical setting with a ruler multiple times was a patient safety concern from the standpoint of potential wound bed contamination, as well as patient comfort. Therefore, only the Scout device measurements had replicate measurements completed. During the conduct of this study, the five readers made three replicate measures for each of the Scout measurements, Scout L×W, and Trace, for each image. Therefore, three replicate measurements are available for each reader for the Scout L×W area, Scout trace area, and Scout trace perimeter.

The readers were trained on the operation of the Scout prior to completing these measurements. Then, each reader completed the Scout L×W and External Wound Trace for each image three times. The Scout L×W is designed to emulate the reference standard ruler technique by taking the greatest length head to toe by greatest width at a 90-degree angle to length. The head orientation was indicated at the time of image capture. When measuring the image, the reader placed the cursor at the head or toe wound edge and drew to the opposing wound edge. The width of the wound was then drawn. The readers were able to use the compass feature of Scout ImageReview to ensure alignment with head orientation relative to each wound.

The External Wound Trace utilizes software to allow the user to visually trace the wound edge. The software then calculates trace area and trace perimeter. Both the Scout L×W and External Wound Trace were completed on the same image.

Wounds were selected from the library of images that met the study criteria. Individual written consent was provided for each wound from each adult 18 years or older. Wounds were excluded if the edges were obscured in any way, if the image was blurred, or if images were not recorded at an 18-inch distance or not at an angle of 90 degrees perpendicular to the external wound. All 40 wounds selected were evaluated for performance on the study device.

To control for carryover, the 40 wound images were randomized. Each reader measured the L×W area, trace area, and trace perimeter for the first set of 40 wound images one time. The reader was then provided with a second set of 40 randomized wound images, with which the reader performed the second set of measurements. This process was repeated for the third set of measurements. A separate randomization was completed for each of the three replicates. The same randomization was used for each of the five readers.

The primary end points for this study were (1) length measure of the wound using the Scout ImageReview software, (2) width measure of the wound using the Scout ImageReview software, (3) calculated square area of the wound using L×W measure of the Scout ImageReview software, (4) surface area of the wound using the External Wound Trace feature, and (5) perimeter of the wound using the External Wound Trace feature.

Data were handled according to the WoundVision, LLC data management procedures. Descriptive statistics included the mean, median, maximum, and minimum for the Scout L×W area and perimeter trace area. Measurements of precision included intrareader and interreader reliability (repeatability), as well as total variability. The CV % was calculated as the SD divided by the mean times 100 for within- and between-readers for each individual wound for the repeatability (reliability) measure. An analysis of variance was completed using a random-effects model with reader and wound in the model as random factors for each measurement method. In addition, the model was rerun including the interaction term as a random factor. The within- and between-reader precision was recalculated separately for the two groups of readers, the three clinical experts, and the two nonexperts. SAS software (SAS Institute, Cary, N.C.) was utilized for statistical analysis.

Objective 1 could not be completed because repeat measurements with the standard of care ruler were impractical. All of the results in this section address objective 2.

Data from all 40 wound images for each of the five readers, with measurements (Scout L×W area and Scout trace area) for each end point completed three times per wound were utilized in analyses. Descriptive statistics are as follows: the average area for the Scout L×W calculation was 20.07 (SD, 1.51) cm² (95% confidence interval, 19.23-20.91 cm²), and the Scout trace area was 16.28 (SD, 1.17) cm² (95% confidence interval, 19.23-20.91 cm²).

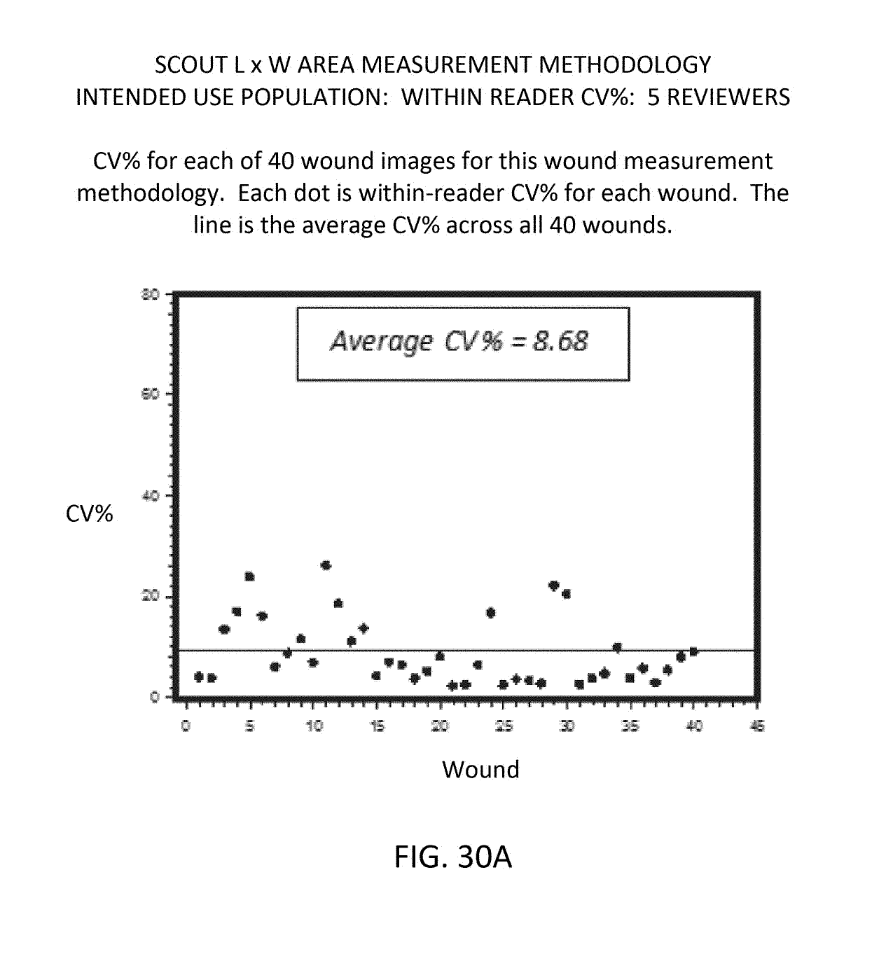

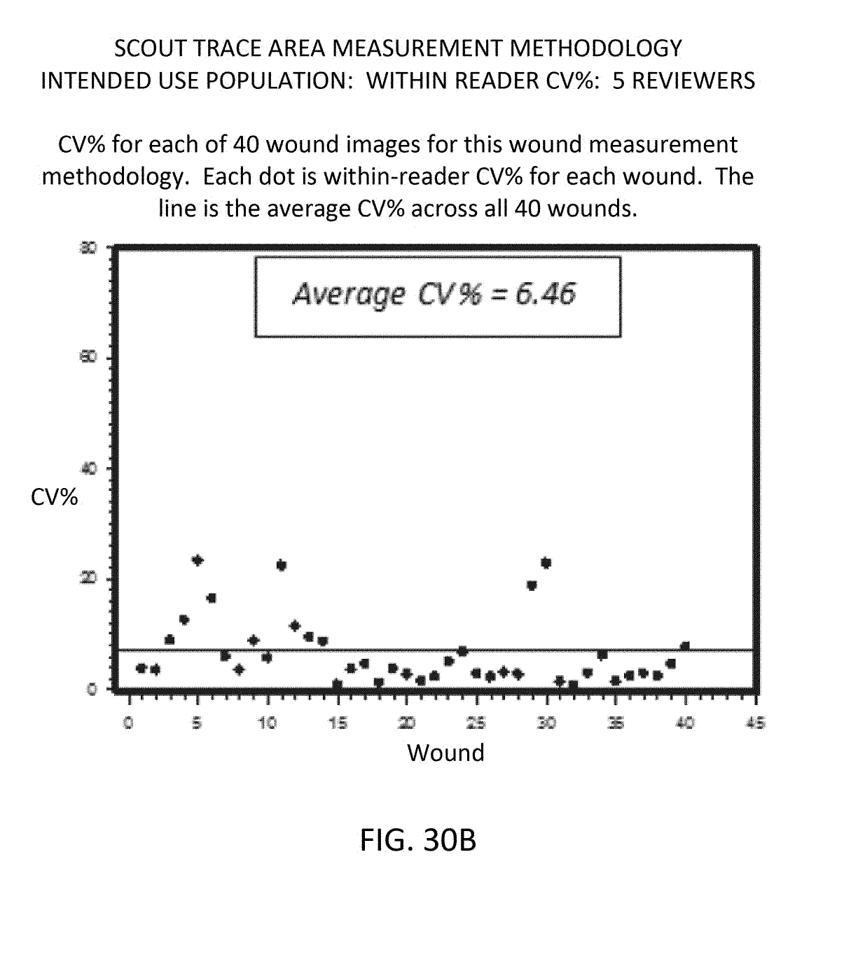

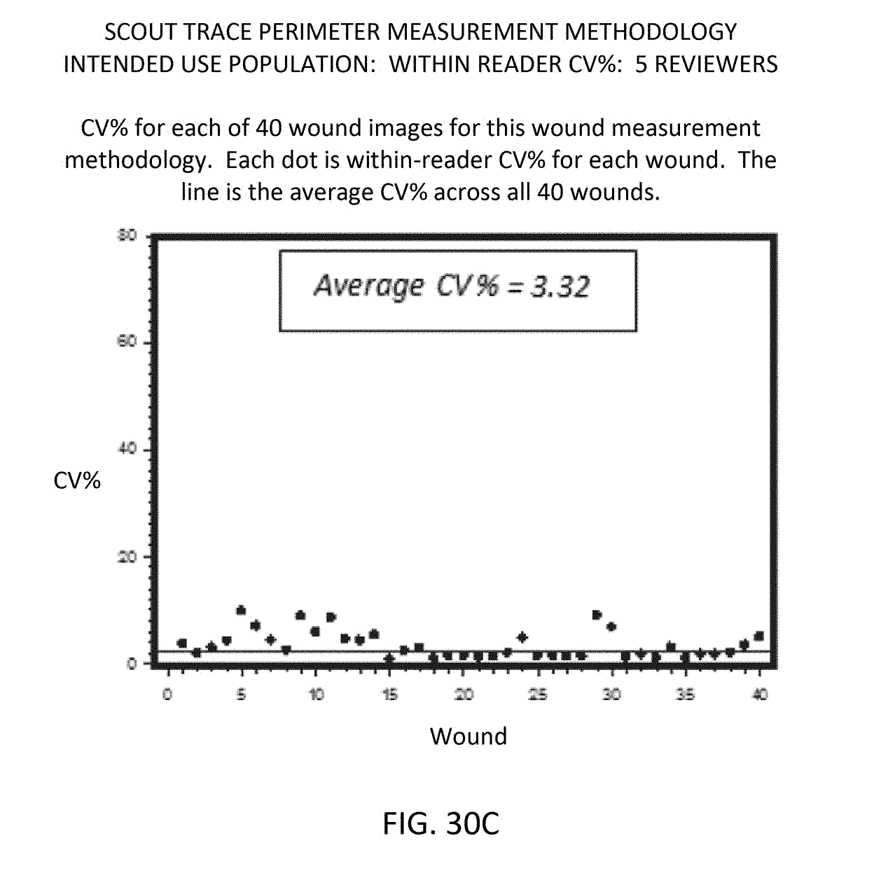

The within-reader precision was calculated for each individual wound and averaged across the five readers for each of the Scout measurement methodologies (FIGS. 30A, 30B and 30C). The average CV % across all 40 wounds was less than 10% for each of the measurement methodologies, with the CV % lowest for the Scout trace perimeter. This suggests that regardless of the measurement used a reader can perform multiple measurements of the same wound with acceptable variation.

RE FIGS. 30A, 30B and 30C: The CV % for each of the 40 wound images for each of the Scout measurement methodologies. Each dot is the within reader CV % for each wound. The line is the average CV % across all 40 wounds for each methodology. Scout L×W area average CV %=8.68; Scout trace area average CV %=6.46, and Scout trace perimeter average CV %=3.32.

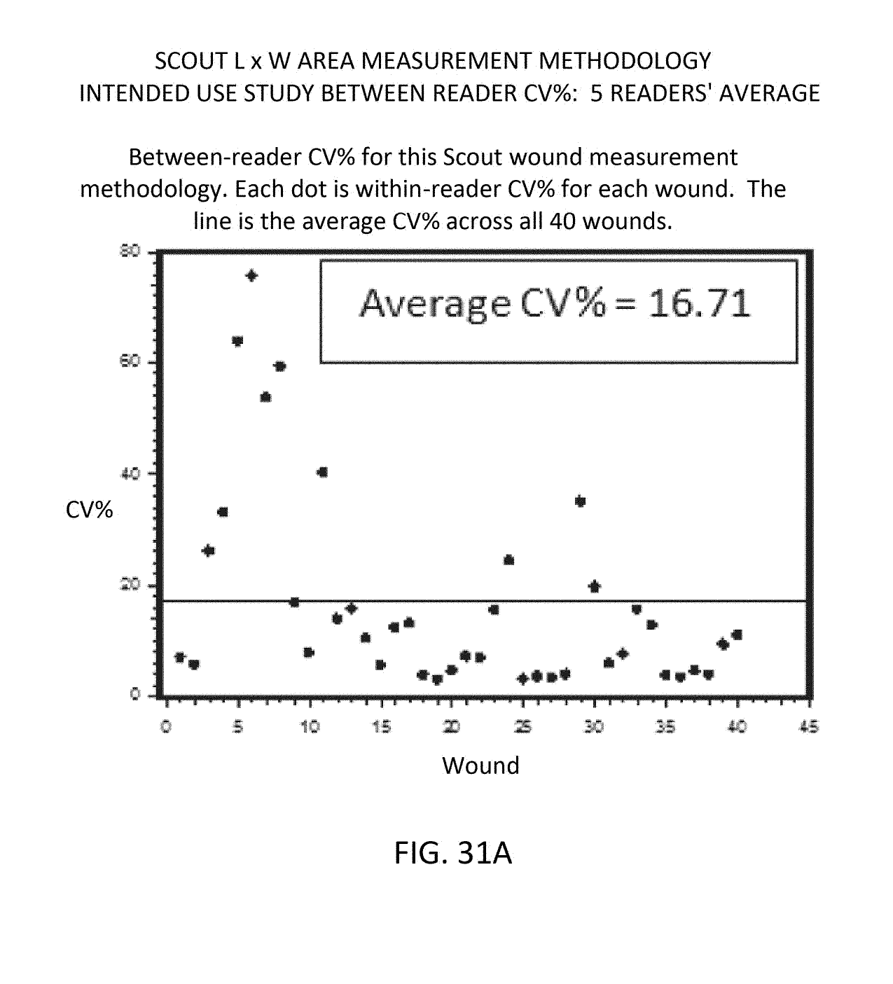

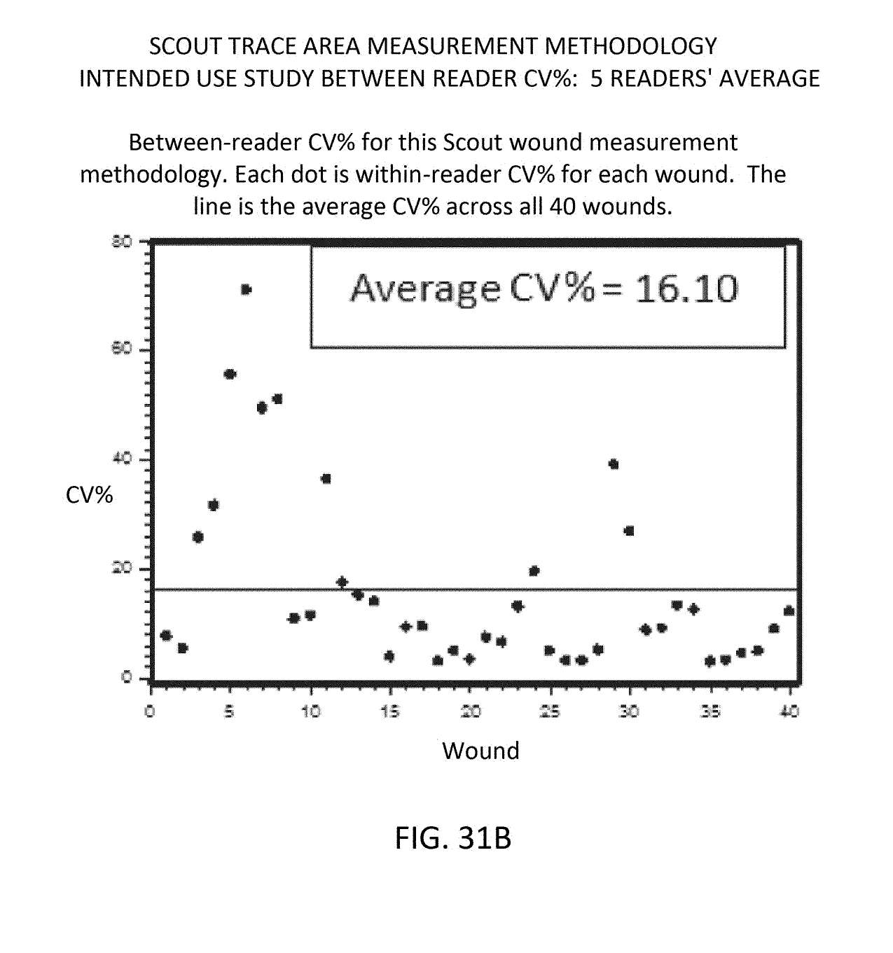

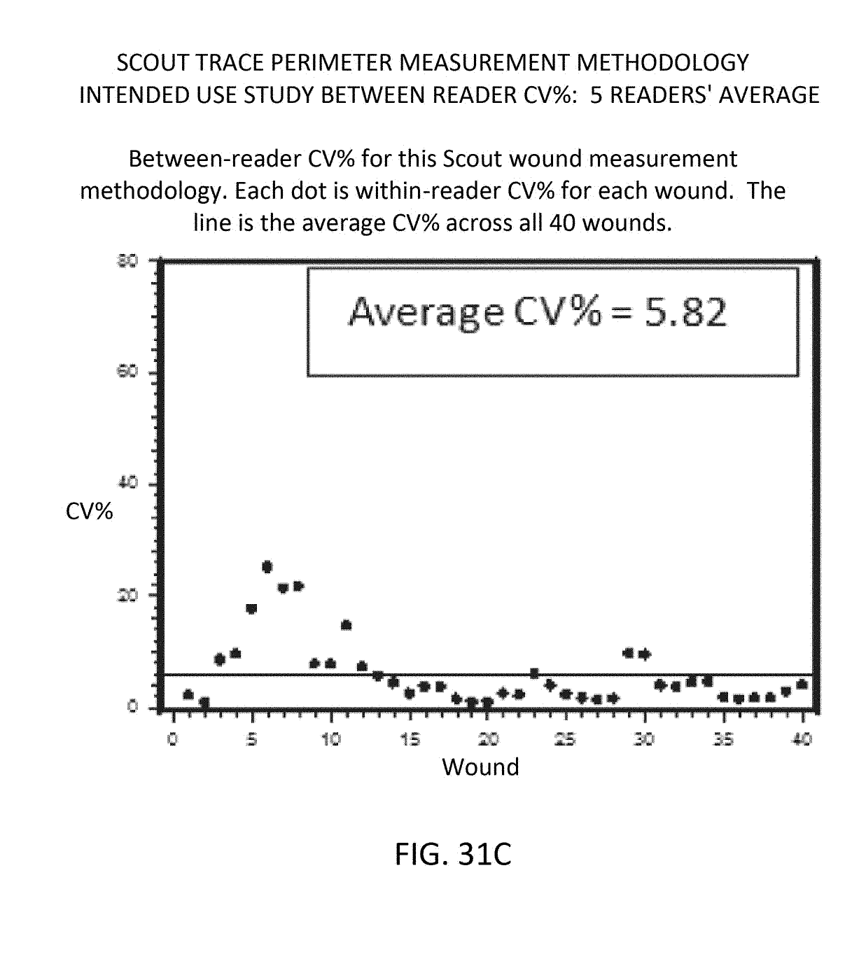

The between-reader precision for each individual wound for each of the Scout measurement methodologies was on average less than 20% CV. Similar to both the within-reader precision and that from a previous study, the average CV % is smallest for the perimeter measurements (FIGS. 31A, 31B and 31C).

RE FIGS. 31A, 31B and 31C: Between-reader CV % for each of the Scout measurements. Each dot is the within reader CV % for each wound. The line is the average CV % across all 40 wounds for each methodology. Scout L×W area average CV %=16.71; Scout trace area average CV %=16.10, and Scout trace perimeter average CV %=5.82.

Data from the study described above in Section I and other literature suggest that when measuring shapes of known size with a defined edge the between-reader agreement shows acceptable variation regardless of measurement technique. The results of this study using actual wounds suggest that regardless of the measurement used, readers differ in how they define the wound's border. The source of this variation may lie within the subjective perception of qualitative wound characteristics. Therefore, from the previous study measuring shapes of known size, in this study measuring actual wounds, as well as the literature, it can be concluded that the differences that exist between readers in wound measurement are not necessarily due to the measurement technique, but rather the judgment of the reader performing the measurement.

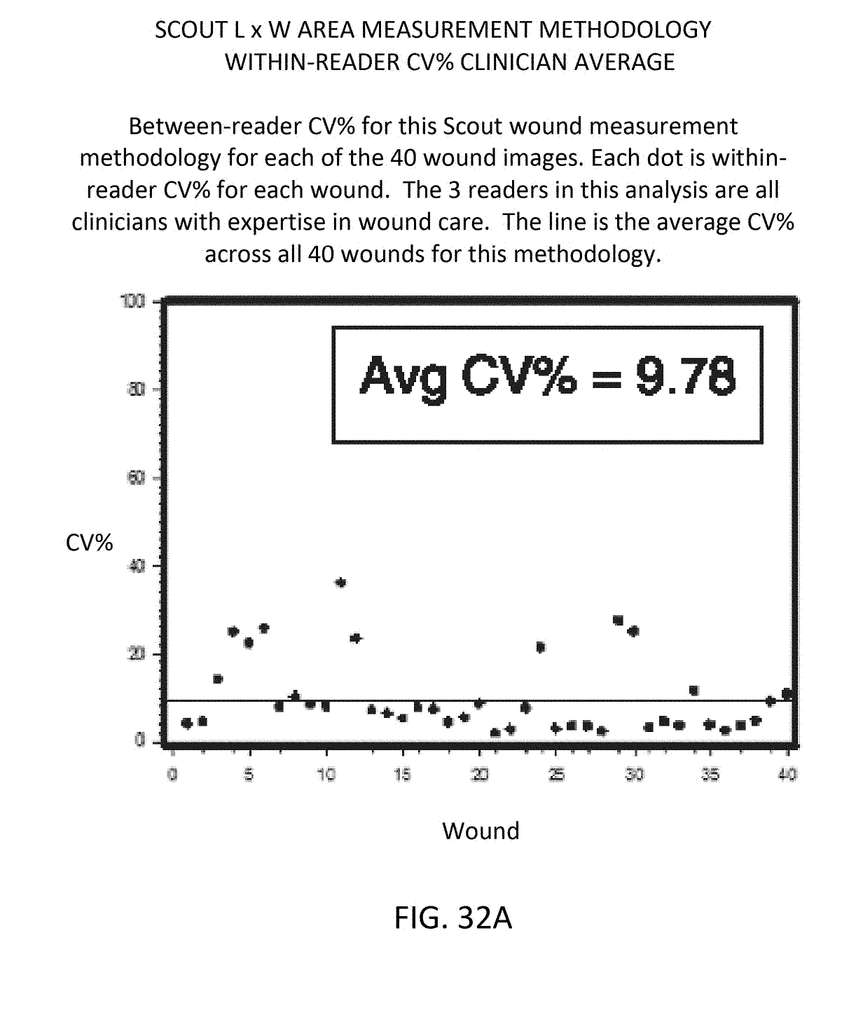

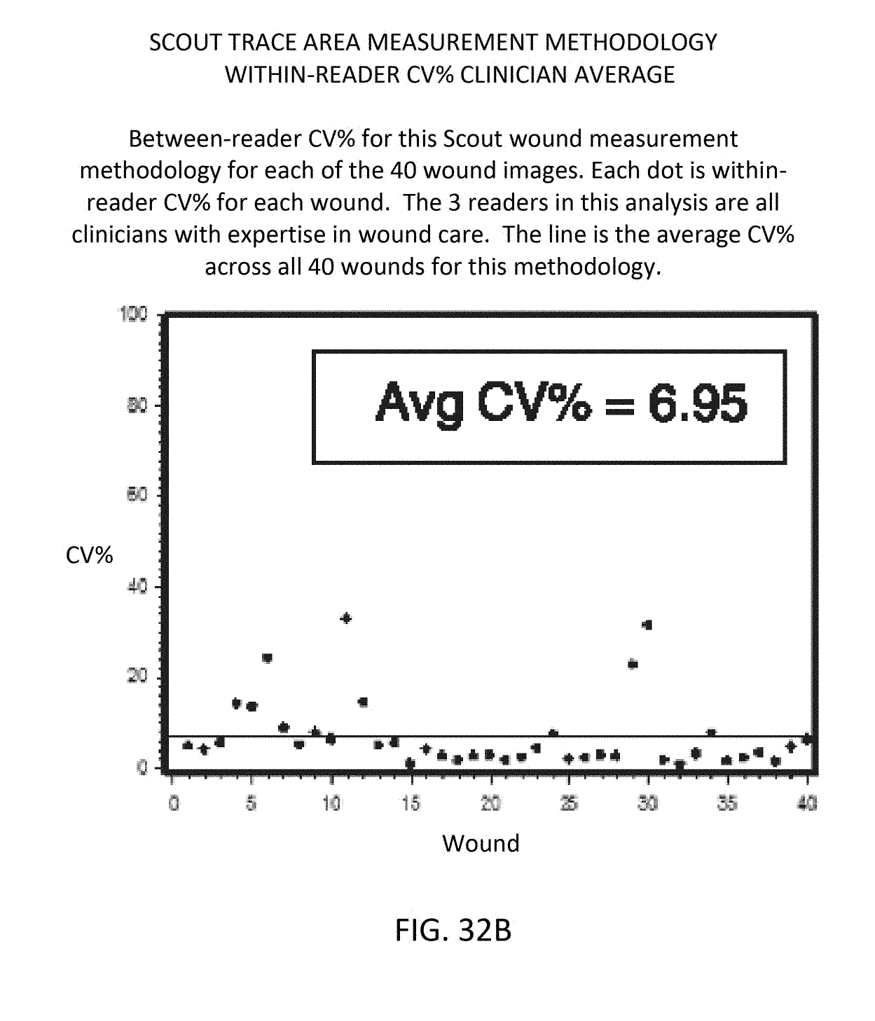

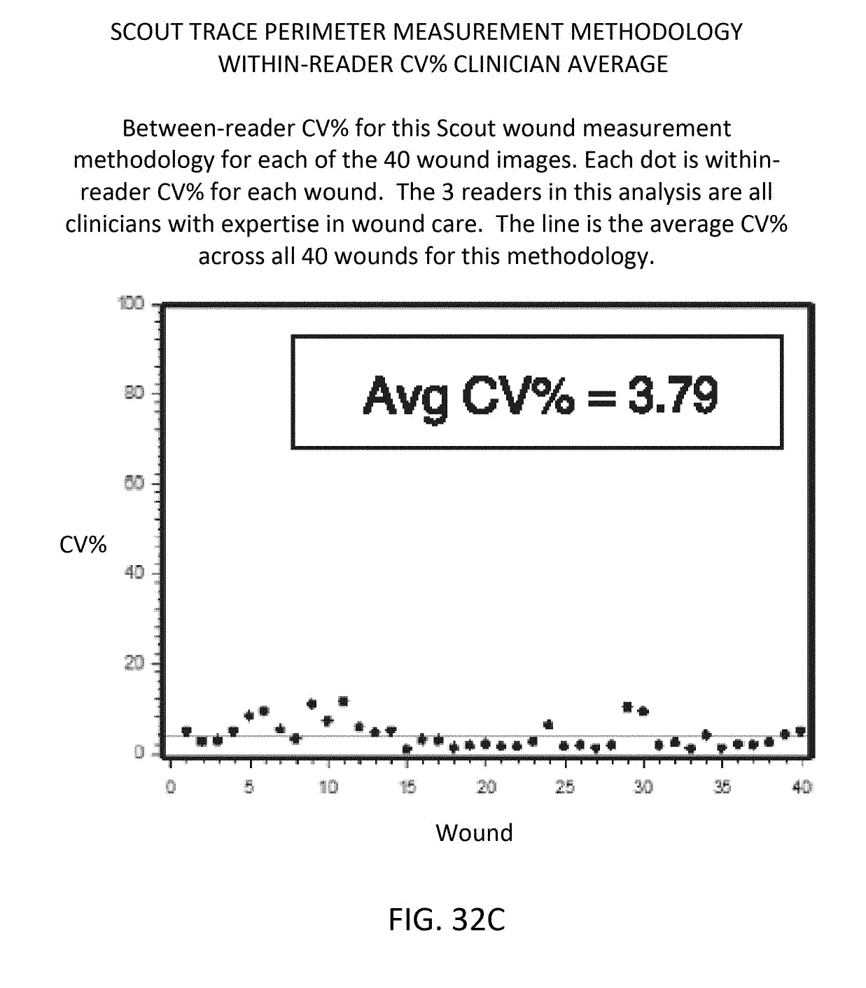

This study used both experts in clinical wound care (n=3) and nonexpert readers (n=2). The within- and between-reader precision for each of the reader types yields similar results (FIGS. 32A, 32B and 32C). These results support that the Scout device can be utilized by a variety of individuals in the clinical setting yielding similar results.

RE FIGS. 32A, 32B and 32C: The within-reader CV % for each of the 40 wound images for each of the Scout measurement methodologies. Each dot is the within-reader CV % for each wound. The line is the average CV % across all 40 wounds for each methodology. Scout L×W area average CV %=9.78; Scout trace area average CV %=6.95, and Scout trace perimeter average CV %=3.79. The three readers in this analysis are all clinicians with expertise in wound care.

Study data suggest that a single reader can measure the same wound multiple times yielding similar results. And as expected, multiple readers do not measure the same wound as well as a single reader. The variation that exists between readers in wound measurement is not necessarily due to the measurement technique but rather the judgment of the reader in determining the wound edges performing the measurement.

The within- and between-reader precision is similar for the Scout trace area (within 6.46 CV % and between 16.10 CV %) and the Scout L×W (within 8.68 CV % and between 16.71 CV %). Perimeter measurement is more precise than both traced area and Scout L×W (within 3.32 CV % and between 5.82 CV %). For all measurements, the within-reader precision is better than the between-reader. For the Scout L×W area, within-reader precision was 8.68 CV % and between 16.71 CV %. For the traced area, within-reader precision was 6.46 CV % and between 16.10 CV %; and for the perimeter, the within-reader precision was 3.32 CV % and between 5.82 CV %.

On analysis of variance when the interaction term was included, there was a significant interaction between wound and reader. However, the wound data are not normally distributed and the within- and between-reader precision is not similar across all wound shapes; therefore, the results of the analysis of variance are not valid.

Study Conclusions:

The within-reader precision was acceptable (CV %<10) for all three measurements (Scout trace perimeter 3.32 CV %, Scout trace area 6.46 CV %, Scout L×W area 8.68 CV %). Although the between-reader variability was larger than the within-reader variability, it still averaged less than 20% for all measurements (perimeter 5.82 CV %, traced surface area 16.10 CV %, and Scout L×W area 16.71 CV %), making it an acceptable technique. This finding suggests that the differences in subjective perception of qualitative wound characteristics, particularly wound edge, can influence wound assessment agreement, consistent with previous literature. The within-reader results using actual wounds in this study are consistent with a previous study on simulated wounds (CV % 3.32-6.68 vs. CV % 2.33-5.39, respectively), demonstrating reliable results from the Scout device in the clinical setting for repeat measurements by the same reader.

Using actual wounds in this study, the between-reader results were greater than those on simulated wounds (CV % 5.82-16.71 vs. CV % 2.75-6.47, respectively) in a previous study. The study described above in Section I used metal objects, obviously enabling a cleaner determination of wound shape or wound edge compared with actual wounds. This finding is consistent with previous research demonstrating that between-reader differences exist less unrelated to measurement technique and more related to the reader judgment of wound edge.

The Scout device provides accurate and reliable measurements of actual wounds. It is most accurate in measuring wound perimeter, even between readers. The current standard of measuring wounds is the L×W area calculation, which is known to have large variability, in fact up to 44%. The Scout device can be used by individuals with varied backgrounds and provides similar results when clinical experts and non-clinicians utilize the device.

The Scout device is able to accurately measure wound perimeter, which is a reliable measurement of wound area. As wounds heal from the bottom up followed by the edges inward, it is a good measure of serial reporting for indications of healing. The device is noncontact; therefore, patient comfort is a nonissue. The Scout device showed 3% variability in the wound shape study, whereas it was only 5% in actual wounds in this current study. The Scout device is reliable in measuring small as well as large wounds.

Techniques for wound measurement that are most desirable are those that are accurate, safe for patients, and easy to learn and use clinically. The technique must also be valid and reliable and sensitive enough to document change over time for clinical as well as research purposes. The noncontact, Food and Drug Administration-approved Scout device meets all these requirements. Although the Scout device is more expensive than a paper ruler, it is far more accurate in documenting progress toward improvement or deterioration of a wound.

K. A Study Regarding Multi-Modality Imaging and Software System for Combining an Anatomical and Physiological Assessment of Skin and Underlying Tissue Conditions;

Timely and accurate assessment of skin and underlying tissue is crucial for making informed decisions relating to wound development and existing wounds. Unfortunately, many drawbacks and limitations are associated with the current, clinically accepted methods for assessment. Current gold standard methods combine a visual assessment of the intact skin at risk or the wound site (wound bed and periwound) with a patient's history and physical. There can never be a replacement for a comprehensive history and physical. However, in order for clinicians to keep pace with the growing burden of wounds, they must adopt new and innovative technologies and techniques to overcome the limitations of the current visual assessment standard, which unfortunately is mostly limited to what clinicians are able to see and do. Visual assessment places clinicians in a difficult situation. Many of the early signs and symptoms associated with wound development and healing present with characteristics that are (a) not visually identifiable until manifestation has occurred, or (b) are difficult to assess with techniques that are largely subjective in nature. Based on new technology, clinicians have the opportunity to take part in a paradigm shift from reactive visual assessment techniques of the past and look ahead to more proactive visual assessment techniques afforded to them by modern day technologies.

This study aims to demonstrate the importance of and limitations to the visual assessment, as well as an emerging technology which can be harnessed to minimize these limitations. In doing so, the assessment of the characteristics relating to wound development and healing is better understood by separating the characteristics into two categories: anatomical and physiological. In regard to wound care, the word assessment can be easily interchanged with the word measurement for the reason that when a clinician is assessing a characteristic they are measuring that characteristic. Fundamentally speaking, by assessing, a clinician is ultimately measuring the presence or absence of a characteristic and/or that characteristic's change over time.

Anatomical assessment is best described as a visual measurement of the structural existence and proportion of features and configurations associated with the disease or injury; i.e. the assessment of a gross anatomy topographic characteristic such as discoloration which is visible to the naked eye.

Physiological assessment is best described as a non-visual measurement of the functional change and development of processes and mechanisms associated with the disease or injury; i.e. the assessment of a thermodynamic characteristic such as temperature which is not visible to the naked eye.

As discussed above, anatomical assessment is limited to what the clinician can see in the visible spectrum, in essence this means clinical recognition and measurement is possible with the naked eye. The visible characteristics include wound size, wound edge definition, tissue type, exudate type and amount, discoloration, and undermining/tunneling. Methods for identification and measurement of these characteristics can be subjective but more importantly they are often times a reflection of what has already happened, leaving clinicians with little or no room for early intervention. Another perspective is to consider it as a measure of the effect from a prior event (cause and effect). An example of this is the ability to identify and measure discoloration/erythema as it relates to suspected deep tissue injury (sDTI) of intact skin and/or the periwound tissue relating to an existing wound, especially in individuals with darkly pigmented skin. Because evolution of sDTI may be rapid and the damage to underlying tissues can manifest before discoloration becomes visually recognizable (topographically present), the identification and measurement of the structural existence and proportion of deep tissue injury via anatomical assessment is impossible. It is imperative that pre-clinical changes such as these are recognized and pressure is relieved before progressing to further damage.

As also discussed above, physiological assessment is limited to what the clinician can touch, smell or hear (from the patient) and is not recognizable in the visible spectrum, in other words meaning clinical recognition and measurement is not possible with the naked eye. These characteristics include temperature, texture, blanchable/non-blanchable erythema, moisture, odor, edema, and pain. All of these characteristics can serve as valuable pre-clinical indicators for the development of non-desirable outcomes before they manifest further (i.e. microperfusion, circulatory impairment, infection or ischemia). Unfortunately, the methods for identification and measurement are not only subjective but inherent difficulties remain in the clinician's ability to identify these characteristics in the first place, making it somewhat of a guessing game. An example of one such limitation is the evaluation of temperature (inflammation or lack thereof) by the method of manual palpation. This method has been shown to be a non-objective means of temperature assessment, even in controlled environments. This method also presents concerns related to cross contamination from continuous contact between a clinician's hand and a patient's body surface.

Although the above examples of anatomical and physiological assessment highlight the limitations of current techniques, all of the methods remain important and serve a purpose. And until easier, more objective methods are developed clinicians must continue to utilize those techniques to the best of their ability. In the interim, it's important that clinicians continue to look for new ways to overcome these shortcomings and embrace new tools and technologies.

It has been shown that anatomical structural imaging (anatomical) combined with analytic software tools can help to decrease the subjectivity and limitations of the anatomical assessment. One such method is improvement of the way in which wound size is measured.

The study described above in Section I stated that a desirable wound measurement technique must not only be accurate, safe, and easy to use but also valid, reliable, and sensitive enough to document change over time. That study was based on the Food & Drug Administration-Cleared Scout device (WoundVision LLC, Indianapolis, Ind.) which met all of the desirable characteristics described above. Study results showed that the Scout device could emulate the L×W measurement with an equal amount of undesired variability (44%). However, the advantage of the Scout device was its ability measure the perimeter of an open wound with very limited variability (5%). The Scout device's precision and accuracy relating to anatomical imaging sets the stage for this study which combines a congruent, functional imaging (physiological) modality with long-wave infrared thermography (LWIT). This allows clinicians to combine an anatomical and physiological imaging tool into their current assessment practices can help to strengthen and empower them with knowledge that is objective, quantitative and otherwise unattainable by current clinical standards. Thermography as a tool for physiological assessment of the skin and underlying tissue is supported by a number of prior studies which suggest that the measurement of temperature can provide a timely and accurate method for monitoring ongoing wound status and could also serve as a useful predictor of wound healing. In regard to wound development, other research has shown that that temperature measurement can assist in the detection of underlying skin necrosis as well as an objective, non-invasive and quantitative means of early deep tissue injury diagnosis.

This study evaluates three aspects of the Scout device's reliability: homogeneity, intrarater reliability, and interrater reliability in an effort to confirm the device's ability to provide clinicians with consistent preclinical and physiologic information that can be incorporated into the current assessment practices.

The Food and Drug Administration-cleared Scout device (WoundVision LLC, Indianapolis, Ind.), previously known as the Wound Measurement and Monitoring System, is a combination digital camera and long-wave infrared camera. The clinician simultaneously captures a visual and infrared image that can be uploaded and stored with a patient's electronic medical record where body surface size and thermal intensity data can be measured and recorded. The digital camera captures the visible light wavelengths from the electromagnetic spectrum which are visible to the human eye. The infrared camera captures the long-wave infrared radiation emitted by the human body from the electromagnetic spectrum (7-14 μm) which is not visible to the human eye.

The Scout's digital camera is indicated for the use of capturing visual images to measure the diameter, surface area, and perimeter of a part of the body or two body surfaces (depth can be acquired manually by the clinician and recorded in the software to calculate to volumetric measurements). The long-wave infrared camera is indicated for capturing thermal images to aid in the measurement of thermal intensity data of a part of the body or two body surfaces. Both components of the Scout are non-contact with respect to the patient and provide an adjunctive tool to help a trained and qualified health care professional measure and record external wound and body surface data. The Scout is considered safe to use (for both patient and user) for capturing both visual and thermal images.

Institutional review board approval was obtained for this study and it was conducted in compliance with the protocol, good clinical practices, and all applicable regulatory requirements. All investigators were trained on the protocol and the proper use of the device and software. There was no anticipated benefit to the study subjects who participated in this study. However, the images collected and results may lead to the improved care in the future.

At the request of the Food and Drug Administration, a number bench tests were required in order to fulfill the Scout device's 510(k) approval. These tests focused on the consistency and sensitivity of the thermographic temperature data provided to clinicians. The bench tests performed are described below.

Bench Test #1: Accuracy of Thermal Image Data Utilizing Scout at Varied Angles

Thermographic images were acquired at multiple angle variances while focused on a calibrated blackbody target. Baseline temperature of the blackbody target was captured at an X, Y coordinate of 0°, 0°. After a baseline was determined, temperature measurements were then captured at eight different angle variances of +30°, 0°; +45°, 0°; −30°, 0°; −45°, 0° and 0°, +30°; 0°, +45°; 0°, −30°; 0°, −45°. The temperature measurements of the multiple angle variances were then compared to baseline to formulate a temperature differential.

There was a minor variation in the thermographic data measured. The average temperature differential across three devices, at all angles was 0.15° C. The largest average variation was seen at angles of 0°, +30° and 0°, −30° which resulted in a 0.22° C. in temperature variation. Since users are instructed to acquire images approximately 90° perpendicular to the body's surface, the data of this bench test suggests that variation of the angle in which users capture data do not affect the sensitivity of the device's thermographic data.

Bench Test #2: Accuracy of Thermal Image Data for Different Infrared Cameras

Three different sample devices acquired one thermographic image every 60 seconds for at least the first 15 minutes and then images can be captured every five minutes. Images were captured for a 45-minute period. This process was repeated three times with the blackbody box set to three different temperatures (26° C., 32° C., and 38° C.) to show that the trend pattern occurs similarly across multiple recorded target temperatures. Minimum, maximum, and mean thermal intensity values were recorded and then plotted to change over time.

The results showed reliability of the Scout to record similar trends between devices. However, due to environmental influences such as room temperature and internal temperature of the device, it was shown that the device cannot accurately capture absolute temperature. The outcome of this test confirms need for the use of relative temperature.

Bench Test #3: Validation of the Scout Device's Conversion of Pixel Value to Celsius

The Scout device can capture up to 254 unique temperature values, also called pixel values. To avoid confusing clinicians with pixel value units, it was important to use a temperature unit most are familiar with. Thus, it was determined converting pixel value to Celsius would be more appropriate.

To validate the accuracy of the Scout's conversion of pixel value into Celsius within a 22-42° C. range, a calibrated blackbody box was set to seven different temperatures within the 20° C. window. The Scout measured these different temperatures in pixel value, converted them to Celsius, and showed a difference in pixel value between each degree ° C. was 12.7 (+/−2 pixels or +/−0.16° C.) throughout the temperature range. Calibrated into a 22-42° C. range, the Scout is sensitive to changes in temperature down to 0.08° C.

Bench Test #4: Effect of Room Temperature on the Scout Device's Thermal Image Data

To determine how environmental temperature affects Scout temperature measurement of a calibrated and unchanging target, the Scout was used in Multiple environments. Temperature measurement was affected by environmental temperature when the image was captured and the amount of the effect could not be conclusively confirmed from the data collected. The outcome of this test confirms need for the use of relative temperature.

Bench Test #5: Accuracy of Thermal Image Data Utilizing Scout at Varied Distances

To determine how distance affects the temperature measurement of a calibrated and unchanging target, a distance test was performed to capture temperature at the suggested distance of 18″ as well as 12″ and 24″. The temperature variation was not greater than a +/−0.5° C. per 6″ of distance change. Further, the largest variation recorded during the test was +0.24° C.

A prospective design was used to retrospectively analyze 40 visual and infrared image pairs of 22 independent wounds. Some of the 40 visual and infrared image pairs were the same wound measured on the same subject at different time points and different stages of healing. Thus, the data set included 22 independent wounds. Because the visual and infrared image pairs of “replicate wounds” were taken at different stages of healing they were deemed independent wounds.

The study objective was to determine within- and between-reader agreement of Scout Visual-to-Thermal Overlay placement (moving the wound edge trace from the visual image onto the wound edge signature of the infrared image).

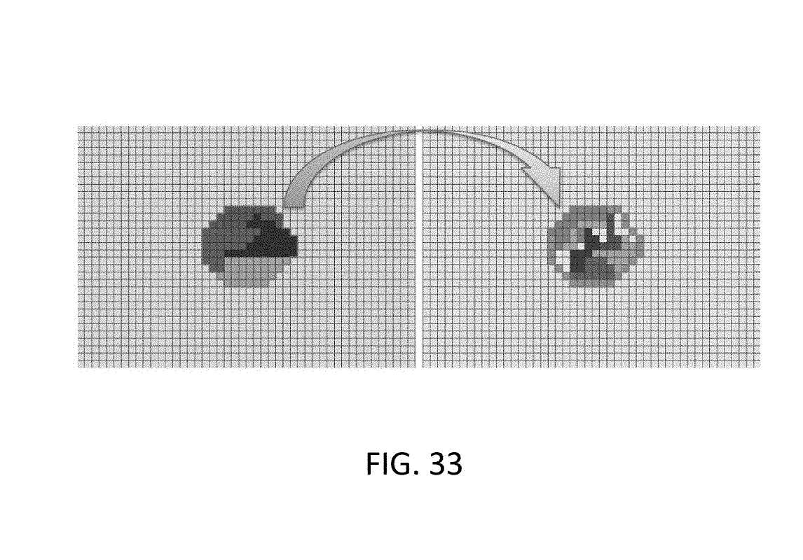

For establishing within- and between-reader agreement of the Scout Visual-to-Thermal Overlay feature, five different readers (two Scout software experts and three wound care experts) overlaid a wound edge trace from the visual image and placed it onto the congruent thermal representation of the wound on a thermal image three independent times (see an illustrative example of a Visual-to-Thermal Overlay in FIG. 33.

RE FIG. 33: Overlaying the wound edge trace from the visual image onto the thermal image the Scout provides a congruent anatomical and physiological measurement of a defined area. By accomplishing this, clinicians have the ability to obtain a measurement of size and temperature that allows them to compare future data with past data.

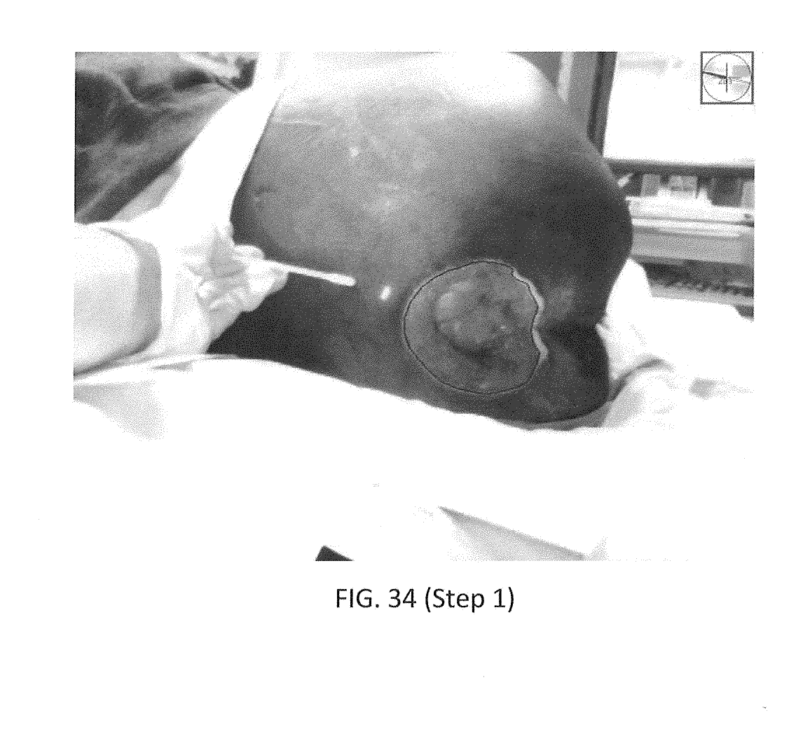

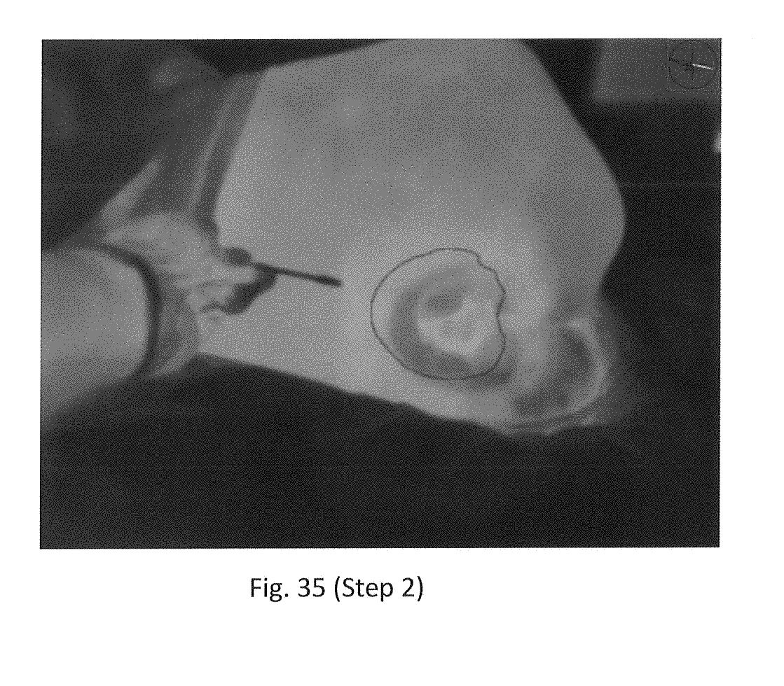

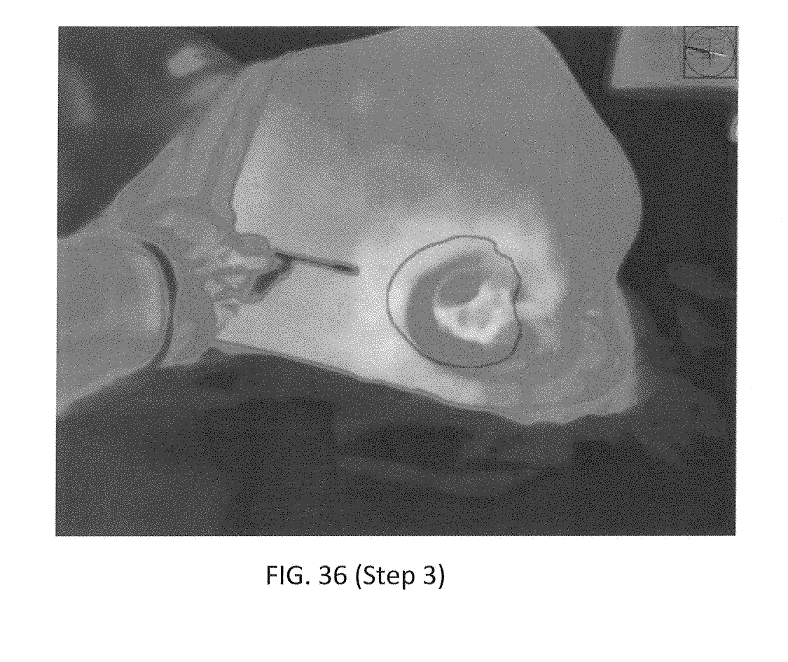

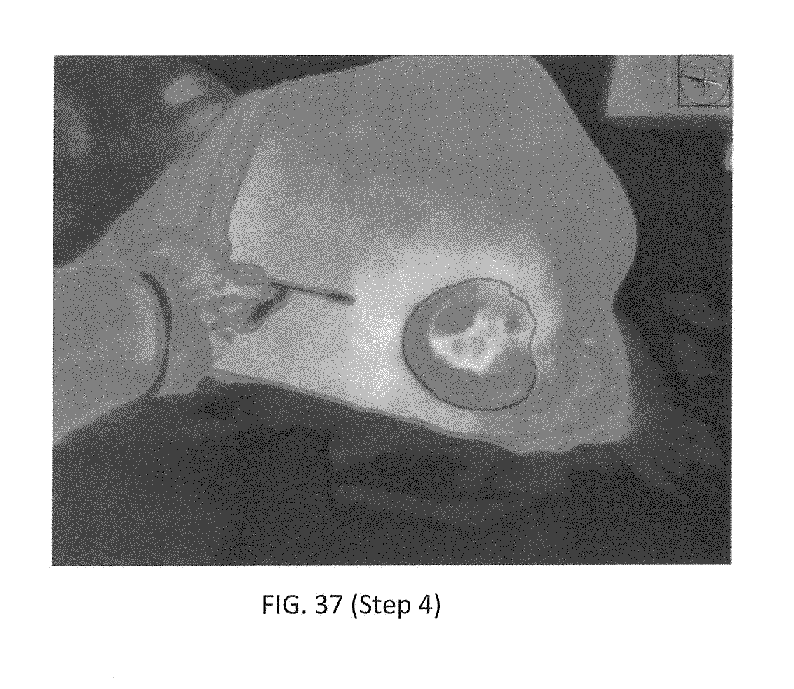

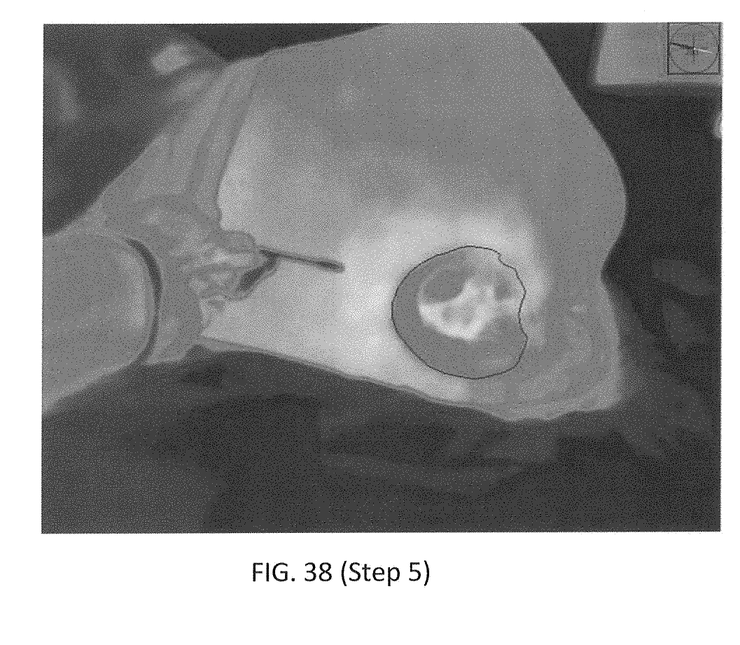

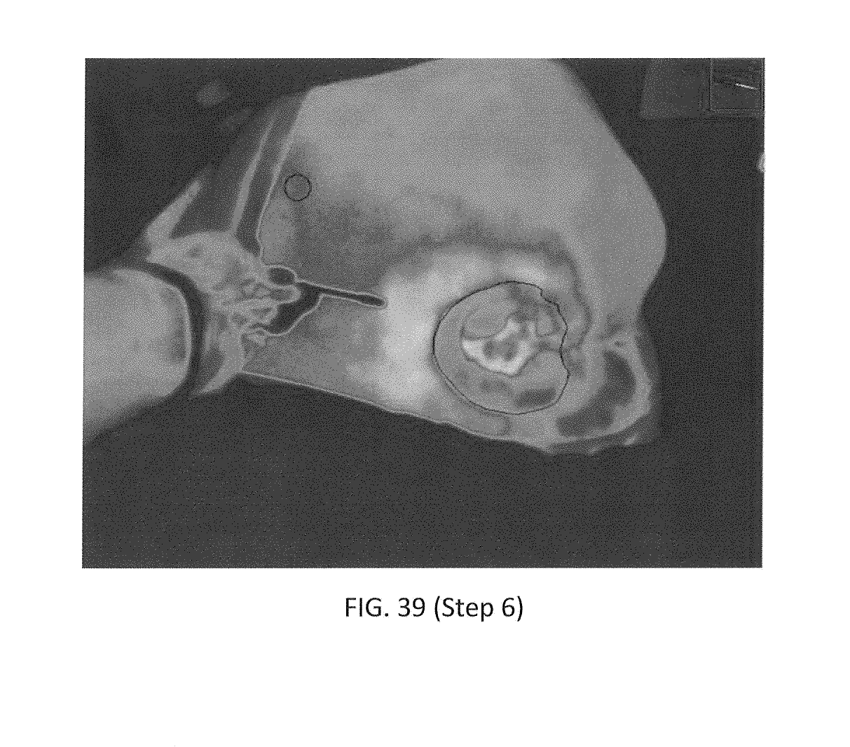

Forty different wound image pairs were evaluated by each reader. Some of the 40 wounds were the same wound measured on the same subject at different time points and different stages of healing. Thus, the data set included 22 completely independent wounds. However, since the “replicate images” were taken at different stages of healing they were considered independent wounds. The wounds were evaluated in a random order both for each user and for each of the three measurements. The step-by-step method for the Visual-to-Thermal Overlay is shown in FIGS. 34, 35, 36, 37, 38 and 39.

Step 1: After a wound edge trace has been completed on the visual image, readers click the Overlay button to superimpose the trace onto the thermal image.

Step 2: The Overlay of the wound edge trace is placed in the center of the GSV thermal image.

Step 3: Readers can toggle to the Color Filter to provide a clearer distinction of the wound edge's signature.

Step 4: The reader drags the Overlay onto the thermal signature of the wound edge.

Step 5: Once satisfied with the position of the Overlay, the reader double-clicks the mouse to place the Overlay. Once the Overlay is placed, the wound edge trace will turn from red to blue and the thermal intensity data can be extracted.

Step 6: While not used in this study, the next logical step in the Scout software process would be to select a Control Area (small circle proximal to wound). This allows for a relative temperature visualization and data extraction.

All readers were trained by the same trainer on the operation of the Scout prior to using the software features. The Scout Visual-to-Thermal Overlay feature is designed to allow clinicians to use an anatomical measurement of the wound on the visual image (area and perimeter) to extract a congruent physiological measurement of the wound on the thermal image (thermal intensity variation data). This is done by taking the wound edge trace from the visual image and overlaying it onto the corresponding thermal signature of the same wound edge. In order to limit the introduction of variability, all three readers overlaid the same wound edge trace. This wound edge trace was completed by one expert Scout software user.

Once an overlay is placed, the software calculates the thermal intensity mean, maximum, and minimum values as well as the total differential (difference between maximum and minimum values). Thermal intensity is calculated in the form of a Pixel Value (PV) from a Grayscale Value (GSV) index which has a range of 1-254. The GSV is a measurement index of thermal intensity which quantifies and visualizes the temperatures differences of the body surface. Darker colors reveal a decrease in the passage of thermal intensity through the tissue (cooler) and lighter colors reveal an increase in the passage of thermal intensity through the tissue (warmer). Each PV represents a percentage of a relative degree in Celsius. A pixel value of 1 is the coolest and a pixel value of 254 is the warmest. The Scout device's thermographic imager is calibrated to identify temperature (thermal intensity) within a calibrated range of 22-42° C. This captures both extremes of the human body's temperature spectrum. PV is to be interpreted as a relative temperature index and it cannot be used as a substitute or comparison to a systemic, absolute measure of temperature.

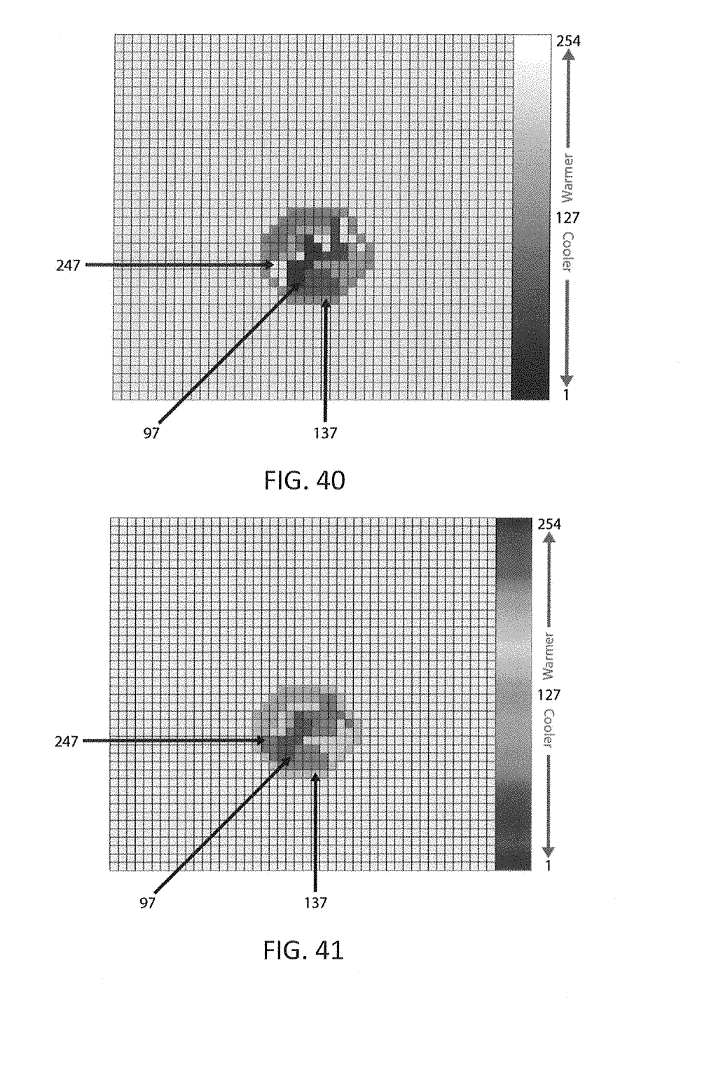

In GSV, a PV of 1 is totally black, a PV of 127/128 is a standard gray (halfway between total black and total white), and a PV of 254 is totally white. Because it is difficult for the human eye to distinguish between 254 shades of gray, the Scout software allows users to apply a Color Filter to the GSV thermal image. Readers had the ability to use this option for easier discernment of the wound's thermal signature (changing the filter doesn't alter the raw PV data). When calculating the thermal intensity data of the Overlay, every single pixel and their respective PVs are factored into the equation. The illustrative example below (FIGS. 40 and 41) highlights three of the 113 pixels and their respective PVs from within the overlay. All of the pixels and their PVs within the Overlay are factored into calculating the end points described in the following section.

RE FIGS. 40 and 41: This illustration shows a thermal image in Raw Grayscale PV (FIG. 40) and Color Filtered PV FIG. 41.

The primary end points are (1) Mean Temperature (the average of all pixel values within the Overlay), (2) Minimum Temperature (the lowest pixel value within the Overlay), (3) Maximum Temperature (the highest pixel value within the Overlay), and (4) Temperature Differential (the difference in pixel value between the high and the low pixel values within the Overlay). These calculations are provided in both PV and Celsius (there are 12.7 pixels per one degree Celsius).

Data were handled according to WoundVision, LLC data management procedures. The statistical analyses were focused on describing the observed within- and between-reader variability for the identification of the Visual-to-Thermal Overlay. Descriptive statistics for all of the outcome measures were completed. In addition, analyses for the data set of 40 wounds and for the subset of the 22 independent wounds were completed. Analyses were also completed for subgroups of the expert readers and non-expert readers.

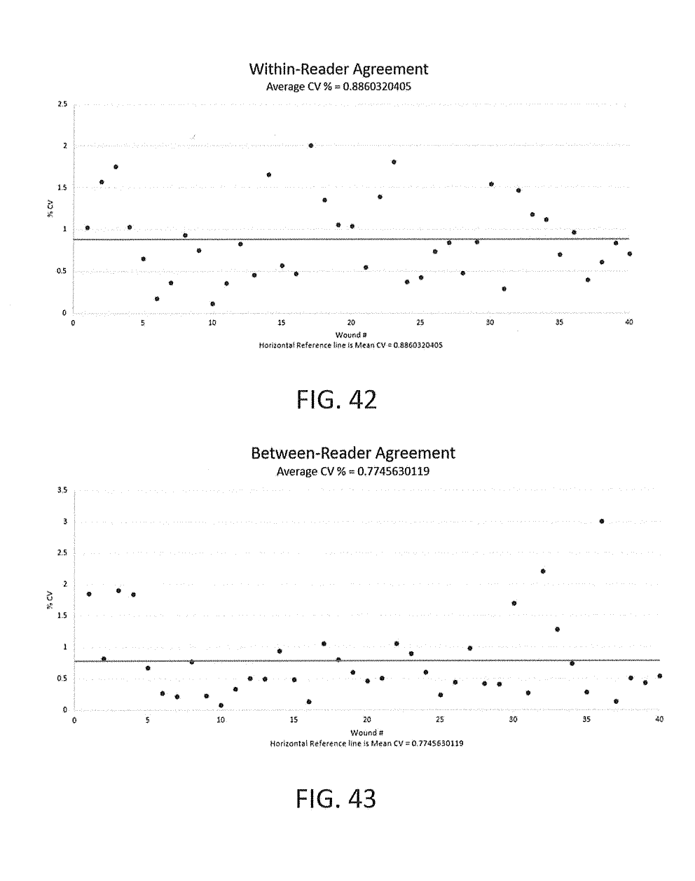

The results are very similar both within and between readers. The coefficient of variation (CV) for the Mean PV both within- and between-readers averages less than 1%, 0.89 and 0.77 respectively (FIG. 42 and FIG. 43). When examined individually, the minimum within-reader percent coefficient of variation was Wound #10, which had a % CV of 0.11. The maximum within-reader percent coefficient of variation was Wound #17, which had a % CV of 2.00.

RE FIG. 42: Within-reader percent coefficient of variation for Mean Temperature averaged across all five readers are shown.

For between-reader, the minimum percent coefficient of variation was Wound #10, which had a % CV of 0.08. The maximum between-reader percent coefficient of variation was Wound #36, which had a % CV of 3.00 (FIG. 43).

RE FIG 43: Between-reader percent coefficient of variation for Mean Temperature averaged across all five readers are shown.

Across all readers and all 40 wounds, the within-reader Mean Temperature was <1 Pixel Value (or 0.08° C.) and % CV was <1.0. Similarly, Maximum Temperature was <1 Pixel Value (or 0.08° C.) and the % CV was <2%.

When converted into degrees Celsius, across all five readers and all three wound replicates the average Temperature Differential is 0.28° C. The largest difference observed was 0.63° C. and the smallest difference observed was 0.04° C.

The Scout software's the Visual-to-Thermal Overlay procedure as implemented in this study, is very precise. All reader measurements were similar and are reproducible both within- and between-readers with a coefficient of variation well below 5%.

The within- and between-reader precision of Mean Temperature measurements are very similar, reflected by an average percent coefficient of variation of 0.89% and 0.77% respectively. The Maximum Temperature average had a coefficient of variation within-reader of 1.68% and between-reader of 1.52%. The Minimum Temperature average had a within-reader coefficient of variation of 0.52% and a between-reader coefficient of variation of 0.35%. The Temperature Differential had a within-reader coefficient of variation of 5.67% and a between-reader coefficient of variation of 5.88%.

No wound measurement varied from minimal to maximum measurements by more than 0.63° C., with the smallest difference observed being only 0.04° C. between the maximum and minimum measurements across all five readers, all three replicates. Across all readers and all wounds, the largest average temperature difference was 0.28° C.

This study demonstrates that the thermal signature of wounds may be delineated repeatedly by the same operator and reproducibly by different operators. Thus, clinicians can integrate a gold standard visual (anatomical) assessment with a congruent physiological assessment to provide them with knowledge relating to presence or absence of blood flow, perfusion, and metabolic activity in the wound, periwound, and wound site.

Study Conclusions:

Temperature is an important albeit underappreciated characteristic in the assessment of wound development and wound evaluation. This under appreciation can be largely attributed to a clinician's inability to identify temperature with ease, accuracy and precision. This study shows how these limiting factors have been overcome and allows for clinicians to harness this data in a way never before possible. The ability to harness temperature data as it relates to the physiology of skin and underlying tissue may offer healthcare providers with a valuable tool for identifying pre-clinical changes associated with wound development and wound healing.

For example, using temperature to assess pressure ulcer development begins with the identification of suspected deep tissue injury (sDTI). sDTI results from the combination of pressure, frictional and shear forces leading to tissue damage. These forces cause soft tissue distortion that leads to reduction of blood flow to an area (ischemia, cell distortion, impaired lymphatic drainage, impaired interstitial fluid flow and reperfusion injury). These pathophysiological changes lead to changes (increase or decrease) of the temperature of the affected tissue, which causes changes of the body surface (skin) temperature. Prior studies suggest that temperature measurement can assist in the detection of underlying skin necrosis and as an objective, non-invasive and quantitative means of early DTI diagnosis.

In regard to temperature and pressure ulcer evaluation, all wound healing is dependent upon vascularization. This translates to perfusion which in turn translates to metabolic activity, ultimately increasing temperature. This increase in temperature is manifested in the form of inflammation or in some situations infection, which can be a barrier to healing. Conversely, when there is no vascularization there can be no perfusion and metabolic activity which ultimately results in a decrease in temperature. This decrease in temperature is manifested in the form of inadequate tissue perfusion or in some situations ischemia, which can also be a barrier to healing as well as tissue necrosis.

The Scout software's ability to provide accurate and reliable quantitative measurements of size (as well as qualitative documentation) through anatomical structural imaging (visual image) is the foundation for obtaining a congruent measurement of temperature through physiologic functional imaging (long-wave infrared thermography). Clinicians now have the option to rely on more than just paper rulers and their naked eye with technologies such as the Scout. By combining the repeatability and reproducibility of the Scout's visual and thermal software measurements, clinicians can now combine clinical judgment with quantitative and objective documentation.

The Scout software application could open the door to a telemedicine approach to wound care. With the number of people age 65 or greater continuing to increase, providers will need to think outside of the box for ways to approach wound care. The ability for clinicians to remotely evaluate skin and wounds using the Scout's visual and thermal images has been proven to provide accurate and repeatable measurements of size and temperature. This quantitative and objective data is also combined with qualitative documentation of skin and wound appearance. The ability for one wound care expert to oversee operations at one or more facilities could not only increase efficiency but also the scope and effectiveness of care that providers can offer.

L. Regarding a Reliability Study Using Long-Wave Infrared Imaging to Identify Relative Tissue Temperature Aberrations of the Body Surface and Underlying Tissue:

Long-Wave Infrared Thermography (LWIT) is a measurement technique that visualizes the thermal energy emitted by the human body surface (also called thermal imaging). Thermal images taken of the skin surface are constructed by passively reading emitted radiant energy formed by the skin and underlying tissue by detecting electromagnetic wavelengths in the long-wave infrared range of 7-14 μm, and then in real time converting these values into pixels within a digital image. The use of LWIT imaging along with visual digital imaging allows both physiologic and anatomic assessment of skin and subcutaneous tissue abnormalities and/or existing open wounds. The physiologic principles assessed by LWIT are based on the body heat produced by cellular metabolism and its distribution by blood to the rest of the body, and particularly to the overlying skin, for loss by radiation and convection. In cases where blood supply is impaired, the impaired areas will show temperature loss due to stunted cellular metabolism. Accordingly, when an area experiences increased or decreased blood supply it will show an increase or decrease of thermal energy which can then be measured by LWIT. The thermal energy being measured by LWIT is converted to a thermal image, from which temperature can be measured.

The importance of LWIT measurement in the assessment of skin and underlying tissues is temperature's direct correlation to the physiological processes of circulation, microperfusion and ultimately metabolic activity. In a healthy human being these physiological processes are regulated to maintain a homeostatic balance. When a stimulus such as a disease mechanism occurs (i.e., ischemia or infection), the body's physiological processes are disrupted, causing them to become pathophysiological in nature. The combination of: a) disturbances caused by the disease mechanism, and b) the body's attempt to control these mechanisms, results in impairment and irregularity thus causing a homeostatic imbalance. The homeostatic imbalance is reflected in aberrations of the desired functions of circulation, microperfusion and metabolic activity which ultimately manifest in the form of changes and irregularities in temperature. Because the changes from the disease mechanisms cannot be seen with the naked eye, temperature measurement (or LWIT) becomes a very important parameter in the physiological assessment of the skin and underlying tissue.

The 2014 International Prevention and Treatment of Pressure Ulcers: Clinical Practice Guideline recommends including assessment of skin temperature in every skin assessment, and particularly so for individuals with darkly pigmented skin. “Localized heat, edema and change in tissue consistency in relation to surrounding tissue (e.g., induration/hardness) have all been identified as warning signs for pressure ulcer development.” An independent review of this guideline revealed that in total there were 822 references to perfusion and circulation, ischemia and necrosis, capillary perfusion and occlusion, oxygenation and hypoxia, and infection and osteomyelitis; all of which have a direct pathophysiological correlation to temperature.

Temperature measurement does have its limitations. In some medical applications, having a single, absolute value for temperature measurement is very useful (for example, a mercury thermometer to measure core temperature). However, when using LWIT to measure temperature of the skin and underlying tissue, clinical application should not focus on absolute temperature value, due to the many intrinsic and extrinsic variables that can affect the ability to capture thermal energy emissivity with 100% accuracy. For example, the intrinsic variables include the normal cycle of thermal production, age, comorbidities, body region, medications, core temperature and others. Extrinsic variables include the ambient temperature, humidity, air convection, climate adaptation of the tissue, configuration of the body surface, substrate temperature of the microbolometer and others.

Because of these variables, a method was developed to identify the quantitative temperature differences that exist in and around a pathophysiological aberration (area of interest being an existing wound or suspected wound) and assess how these temperature differences change over time. In order to achieve this, the aforementioned variables must be minimized. The concept of minimizing these intrinsic and extrinsic variables is referred to as relative temperature differential (RTD). To quantify and achieve RTD measurement, a control area must be selected. A control area, in this example, is defined as a regional, adjacent area of intact tissue (or of similar proximity on the contralateral body region) believed to be least affected by a pathophysiological aberration. The purpose of RTD and a control area selection is to provide clinicians with repeatable and reproducible data to assess circulation, microperfusion and metabolic activity of a pathophysiological aberration relative to an unaffected control area.

For example, a clinician wishes to assess a patient's lower extremity wound using LWIT in an attempt to identify an increase or decrease in perfusion and blood flow in response to a treatment. Comparing absolute temperature measurements of the lower extremity wound at a baseline encounter and a follow-up encounter would provide the clinician with incomparable and unreliable data. This is because there is no way to minimize the variables that could have an effect on the wound's temperature on any given day (for example, on the day of the follow-up the room could be warmer as compared to the day of baseline). However, by selecting a control area the data can be normalized and compared from one moment in time to another. This is because the control is exposed to the same intrinsic and extrinsic variables as the wound, thus providing the clinician with an RTD measurement. By utilizing RTD, all intrinsic and extrinsic variables can be accounted for and the clinician can longitudinally compare RTD change through ratio analyses and other normalization algorithms that account for the variables present a given moment in time.

Achieving RTD in a repeatable and reliable fashion is imperative. Thus, it is important that clinicians utilizing LWIT are able properly select a control area. This study evaluates two aspects of the LWIT device's reliability: (1) Within and Between-reader Agreement of Initial Patient Encounter Images; and (2) Between-Reader Agreement of Follow-Up Encounter Images. Achieving RTD via selection of a control area through a reliable methodology can provide clinicians with valuable data that they otherwise would have no ability to obtain when assessing suspected wounds and the status of existing wounds. By demonstrating the reliability of RTD measurement using the FDA-cleared visual and LWIT imaging device and software analysis tool called the Scout (WoundVision LLC, Indianapolis, Ind.) this study will help confirm its ability to provide clinicians with a reliable and reproducible tool to incorporate into clinical assessment.

The Food and Drug Administration-approved Scout device (WoundVision LLC, Indianapolis, Ind.), known as the Scout, is a combination digital camera and long-wave infrared camera. The Scout enables the clinician simultaneously to capture a visual and infrared image that can be uploaded and stored with a patient's electronic medical record. Body surface size and thermal intensity data can be measured and recorded. The digital camera captures the visible light wavelengths from the electromagnetic spectrum which are visible to the human eye. The infrared camera captures the infrared radiation emitted by the human body from the electromagnetic spectrum which is not visible to the human eye.

The Scout's digital camera is indicated for the use of capturing visual images to measure the diameter, surface area, perimeter, and volume of a part of the body or two body surfaces. The long-wave infrared camera is indicated for the use of capturing thermal images to measure the thermal intensity data of a part of the body or two body surfaces. Both components of the Scout are non-contact with respect to the patient and provide an adjunctive tool to assist a trained and qualified health care professional in measuring and recording external wound and body surface data. The FDA-approved Scout is safe to use (for both patient and user) for capturing both visual and thermal images.

This study was Institutional Review Board approved and was conducted in compliance with the protocol, good clinical practices, and all applicable regulatory requirements. All investigational staff members were trained on the protocol and the proper use of the device and software. There was no anticipated benefit to the study subjects who participated in this study. However, the images collected may lead to the improved care in the future.

The accuracy and reproducibility of the Scout's ability to enables clinicians to assess wounds and wound development from an anatomic and physiologic perspective has been examined in previous studies. When measuring wound size through the visual images (anatomic assessment), the device examined was proven to be accurate, clinically feasible, safe for patients and easy to learn and use clinically. These wound measurement techniques (Length by Width, Surface Area and Perimeter) were also proven to be valid and reliable and sensitive enough to document change over time for clinical as well as research purposes. In an attempt to combine two separate imaging modalities, a separate study examined the ability to mirror the precision and accuracy of the visual imaging modality's measurement of size with the LWIT imaging modality's (physiologic assessment) measurement of temperature in a congruent fashion. This study proved the device is very precise in measuring temperature via a method of combing both of these modalities. This method is reproducible both within and between-readers.

The studies mentioned above prove the device's ability to combine the visual and LWIT measurements (anatomic and physiologic assessment). This allows clinicians to combine clinical judgment with quantitative and objective documentation of wound size and temperature. And by incorporating an anatomical and physiological imaging tool into current assessment practices it can help to strengthen and empower clinicians with knowledge that is objective, quantitative and otherwise unattainable by current clinical standards.

A prospective design was used to retrospectively analyze 102 previously collected visual and infrared image sets of 26 completely independent wounds. The 102 visual and infrared image sets consisted of 26 image sets collected from an initial patient encounter and 76 image sets from follow-up encounters for longitudinal evaluation. Each of the 76 follow-up images were taken at a different point in healing. Thus, the 102 image sets created 26 unique wound encounters and 26 longitudinal wound evaluations.

This study had two primary objectives. The first objective was to establish within and between-reader agreement of the Scout's Control Area placement (selection of adjacent, intact area of tissue on the thermal image to convert raw temperature data to relative temperature data) on a thermal image from an initial patient encounter (no Control Area selection is available for view to the reader). The second objective was to establish between-reader agreement of the Scout's Control Area placement (selection of adjacent, intact area of tissue on the thermal image to convert raw temperature data to relative temperature data) on a thermal image from follow-up patient encounters (Control Area selection from the previous patient encounter is available for view to the reader).

Within and Between-Reader Agreement of Initial Patient Encounter Images:

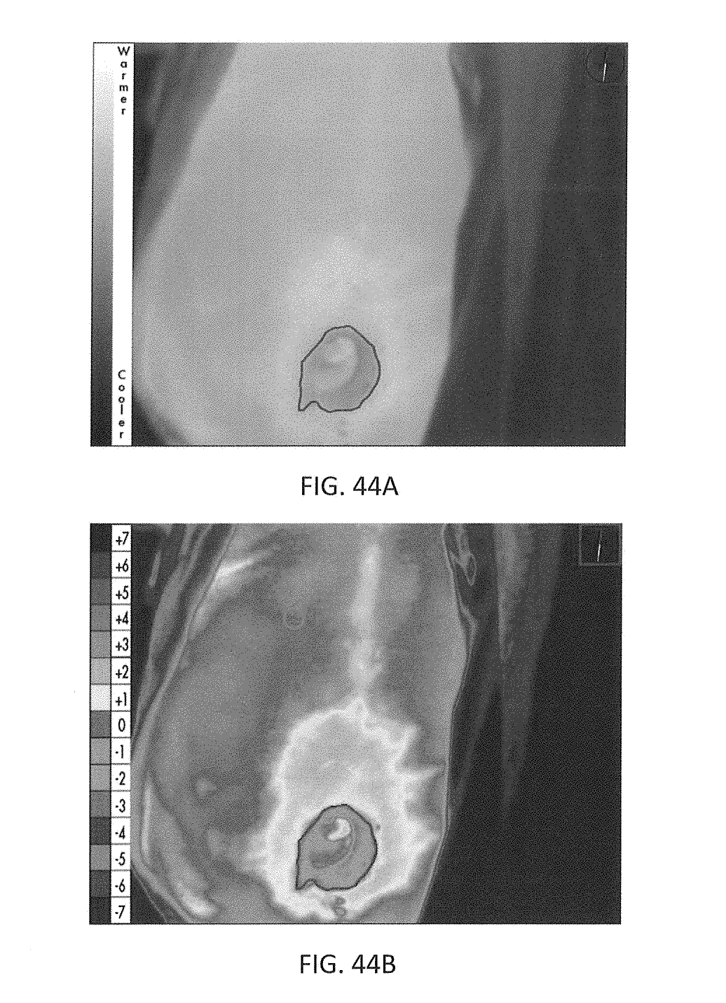

For establishing (A) within-reader agreement (intrarater reliability) of the Scout's Control Area placement from an initial patient encounter, three different readers were asked to place a Control Area on each of the 26 independent wound image sets three separate times for a total of 78 independent placements. For establishing (B) between-reader agreement (interrater reliability) of the Scout's Control Area placement from an initial patient encounter, three different readers were asked to place a Control Area on each of the 26 independent wound image sets for a total of 26 independent placements. FIGS. 44A and 44B shows an exemplary image on which readers were to select Control Area placement.

RE FIGS. 44A and 44B: Shown is an example of initial patient encounter for Control Area Selection. The grayscale thermal image (absolute temperature) of FIG. 44A is exemplary of the 26 images that readers were presented with in order to choose a Control Area. The color thermal image (relative temperature) of FIG. 44B is a result of Control Area selection and a transition from absolute temperature to relative temperature.

Between-Reader Agreement of Follow-Up Encounter Images:

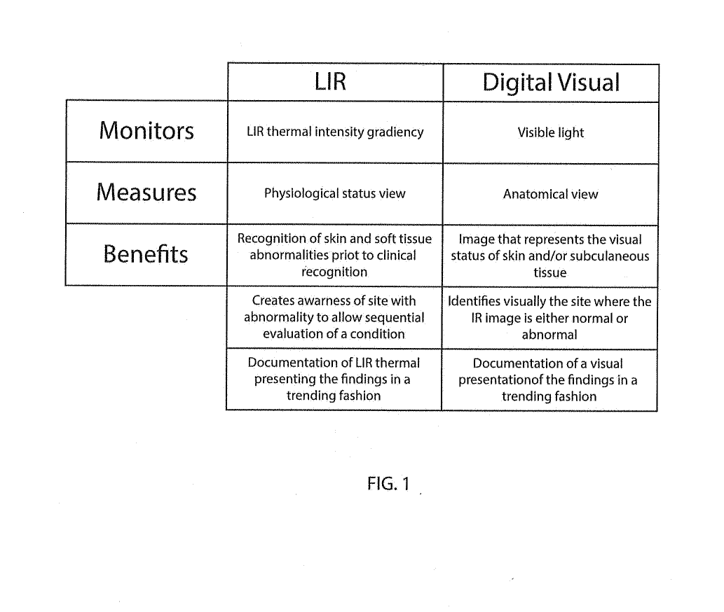

And to establish (C) between-reader agreement (intrarater reliability) of the Scout's Control Area placement from follow-up patient encounters, three different readers were asked to place a Control Area on each of the 76 follow-up image sets for a total 26 longitudinal wound evaluations. FIGS. 45A-1, 45A-2; 45B-1, 45B-2; and 45C-1, 45C-2 show an exemplary longitudinal image set from three patient encounters where readers were asked to place a Control Area based on their selection in the prior encounter. The increased ease-of-use for interpreting thermal images by switching from an absolute image to a relative image can also be seen in this example.

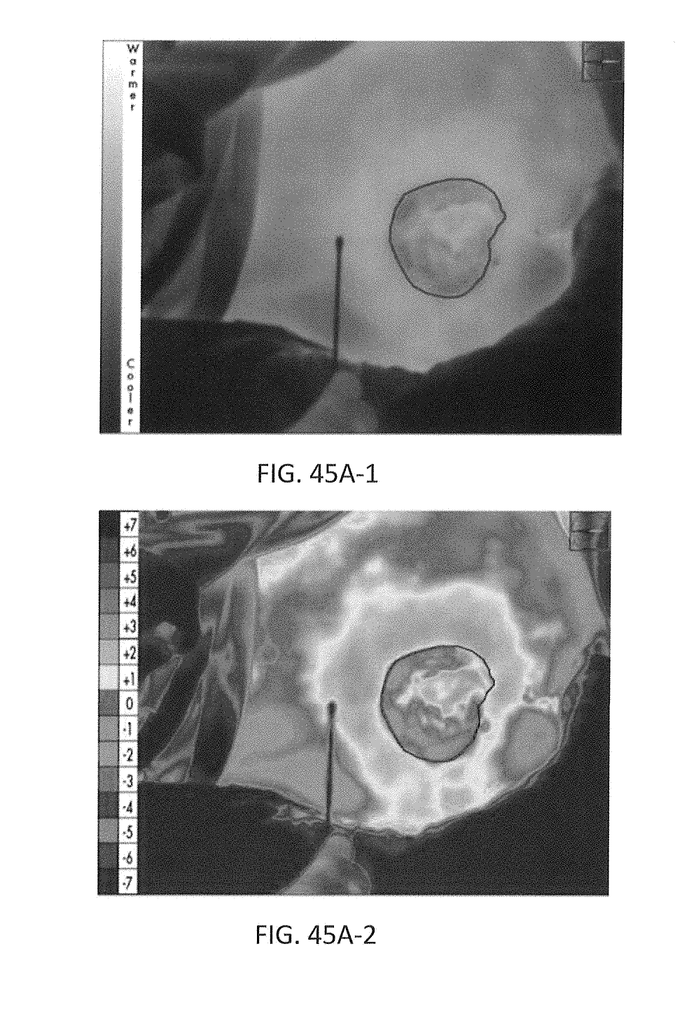

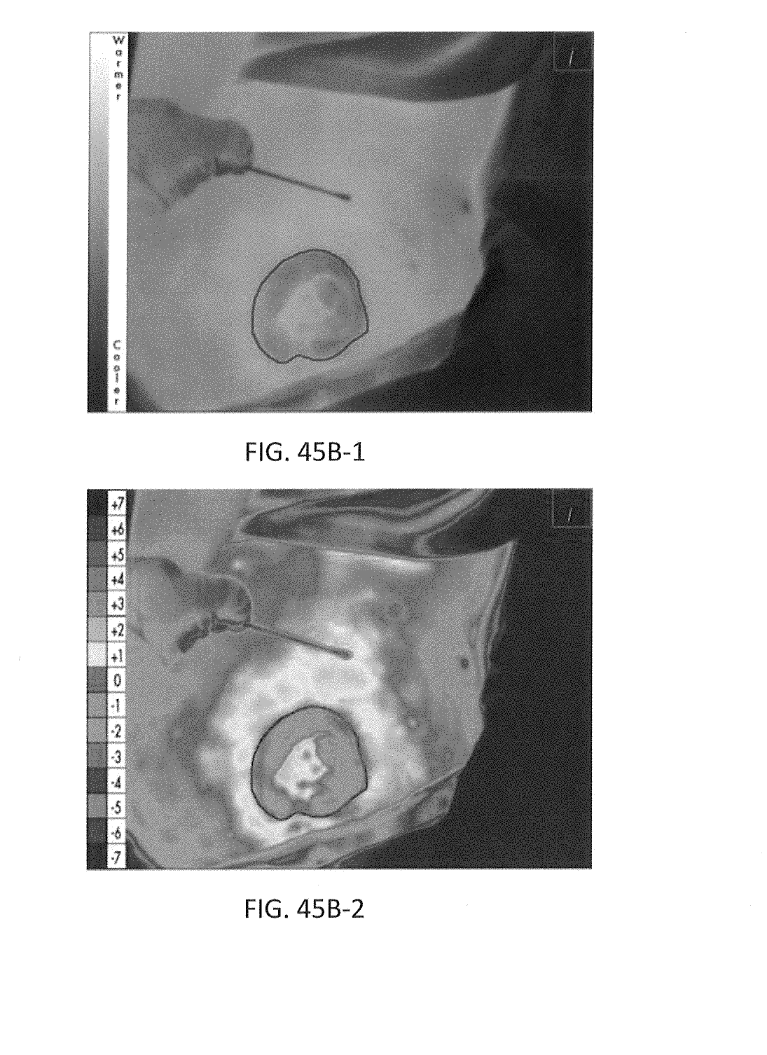

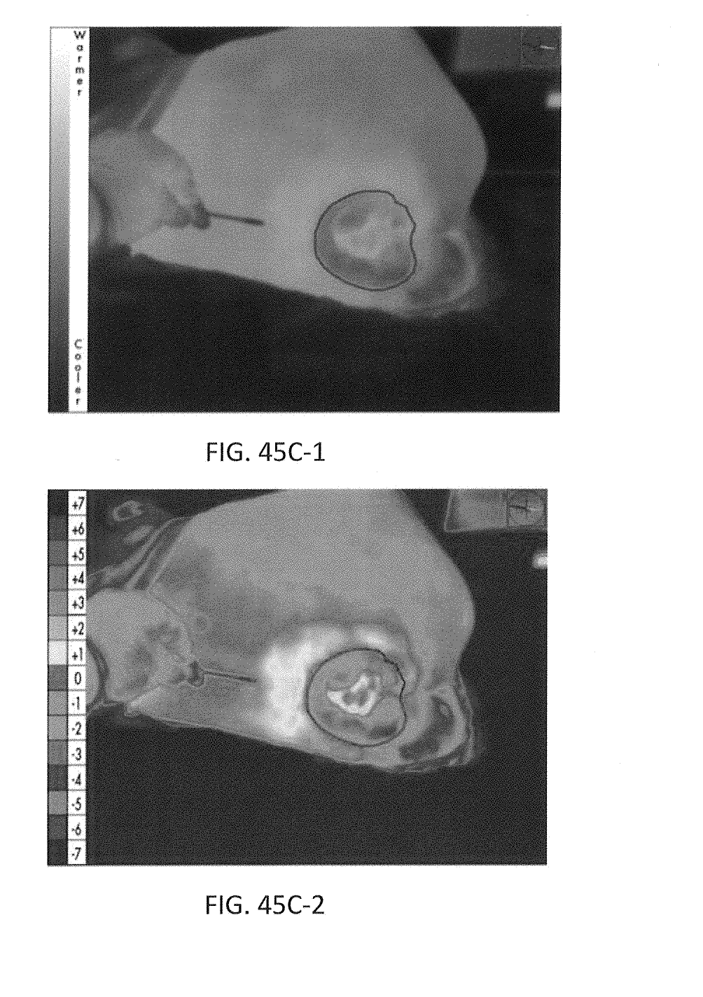

RE FIGS. 45A-1, 45A-2; 45B-1, 45B-2; and 45C-1, 45C-2: The grayscale thermal images (absolute temperature) of FIGS. 45A-1, 45B-1 and 45C-1, are images that readers were presented with before choosing a Control Area. The color thermal images (relative temperature) of FIGS. 45A-2, 45B-2; and 45C-2 are the respective results of Control Area selection and transition from absolute temperature to RTD.

All readers were trained on the operation of the Scout prior to using the software features. The Scout Control Area feature is designed to provide users with relative temperature data as an alternative to absolute temperature data. Users were trained on proper selection of a Control Area which is defined as the selection of adjacent tissue (or in some cases contralateral tissue) on the thermal image that does not show signs of wounding. Adjacent tissue is defined as tissue that does not show signs of wounding but is in the same anatomical region as the wound and periwound. In other words, a Control Area is selected on adjacent, intact tissue to create a baseline that compares the viable tissue to the vulnerable tissue (healthy (good) vs. unhealthy (bad)). To select a Control Area, a user places a small circle (Control Area) onto the tissue they believe is representative of the best comparator. The size of the Control Area is a 438 pixel circle (approximately a 1.5 centimeter diameter). After selection of a Control Area, a mean temperature value is calculated based on the 438 pixels within the circle.

Selection of a Control Area accomplishes two important things. First, it makes interpretation of the thermal image easier through the creation of more defined distinctions and a simpler color palette. Secondly, it minimizes the intrinsic and extrinsic temperature variables associated with absolute temperature. Intrinsic variables include the normal cycle of thermal production, age, comorbidities, body region, medications, core temperature, etc. Extrinsic variables include the ambient temperature, humidity, air convection, climate adaptation of the tissue, configuration of the body surface, substrate temperature of the microbolometer, etc. Eliminating these variables and shifting from absolute to relative temperature allows for longitudinal comparison of the area of interest in the form of images, graphs and quantitative data. A longitudinal comparison allows clinicians to assess circulation, perfusion, and metabolic activity change over time and adjunctively incorporate it into their clinical decision making.

The primary endpoint is Mean Temperature (Pixel Value or Degree Celsius Value), which is defined as the average of all pixel's temperature value within the Control Area; and 12.7 pixels values is equivalent to 1 degree Celsius.

Data were handled according to WoundVision, LLC data management procedures and the statistical package used was SAS. The statistical analyses were focused on describing the variability observed within and between users for the identification of the Control Area placement on the thermal image. For establishing (A) within-reader and (B) between-reader agreement of Scout's Control Area placement from an initial patient encounter, descriptive statistics were used. The descriptive statistics included mean, variance, standard deviation, and percent coefficient of variation over all the wounds and by operator for each wound.

Within-reader agreement is defined as each wound measured three times for each operator independently. Between-reader agreement is defined as the average for each operator compared to the other operators for each wound. For establishing (C) between-reader agreement of Scout's Control Area placement from follow-up patient encounters, descriptive statistics were used. The descriptive statistics included mean, variance, standard deviation, and percent coefficient of variation over all the wounds and by operator. Between-reader agreement is defined as the Mean Pixel Value/Degree Celsius for each operator compared to the other operators for each wound.

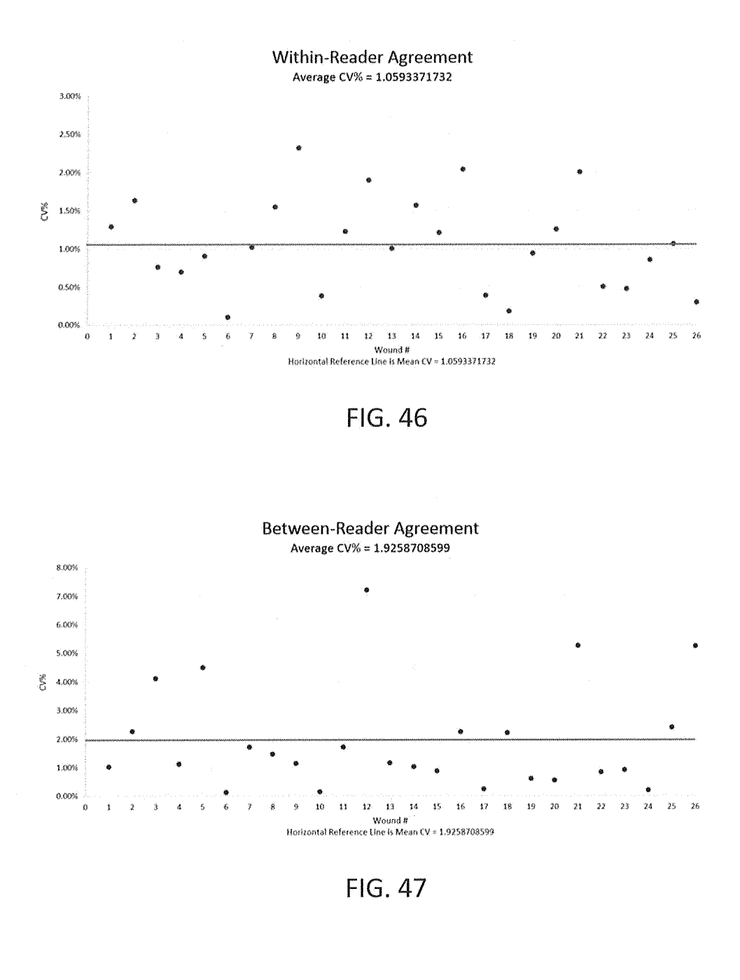

The results are very similar for both within- and between-readers. The coefficient of variation (CV) for the Mean Temperature both within and between-readers averages less than 2%, 1.06 and 1.92 respectively (FIG. 46 and FIG. 47), as indicated by the horizontal lines.

RE FIG. 46: Within-reader Percent Coefficient of Variation for mean temperature averaged across all three Readers are shown.

RE FIG. 47: Between-reader percent coefficient of variation for mean temperature averaged across all three readers are shown.

When examined individually, the minimum within-reader percent coefficient of variation was 0.10, while the maximum within-reader percent coefficient of variation was 2.32. For between-reader, the minimum percent coefficient of variation was 0.13, and the maximum between-reader percent coefficient of variation was 7.19.

As shown in FIG. 46, the within-reader percent coefficient of variation for Mean Temperature is 1.06%. The minimum observed percent coefficient of variation was 0.10% and maximum was 2.32%. The average difference in Mean Temperature within-readers is 1.79 Pixel Values (or 0.14° C.). The minimum observed average difference in Mean Temperature is 0.07 Pixel Values (or 0.01° C.) and the maximum is 8.60 Pixel Values (or 0.68° C.).

Also shown in FIG. 47, the between-reader percent coefficient of variation for Mean Temperature is 1.93%. The minimum observed percent coefficient of variation was 0.13% and maximum was 7.19%. The average difference in Mean Temperature between-readers is 3.70 Pixel Values (or 0.29° C.). The minimum observed Mean Temperature difference is 0.33 Pixel Values (or 0.03° C.) and the maximum is 12.24 Pixel Values (or 0.96° C.).

Between-Reader Agreement of Follow-Up Encounter Images:

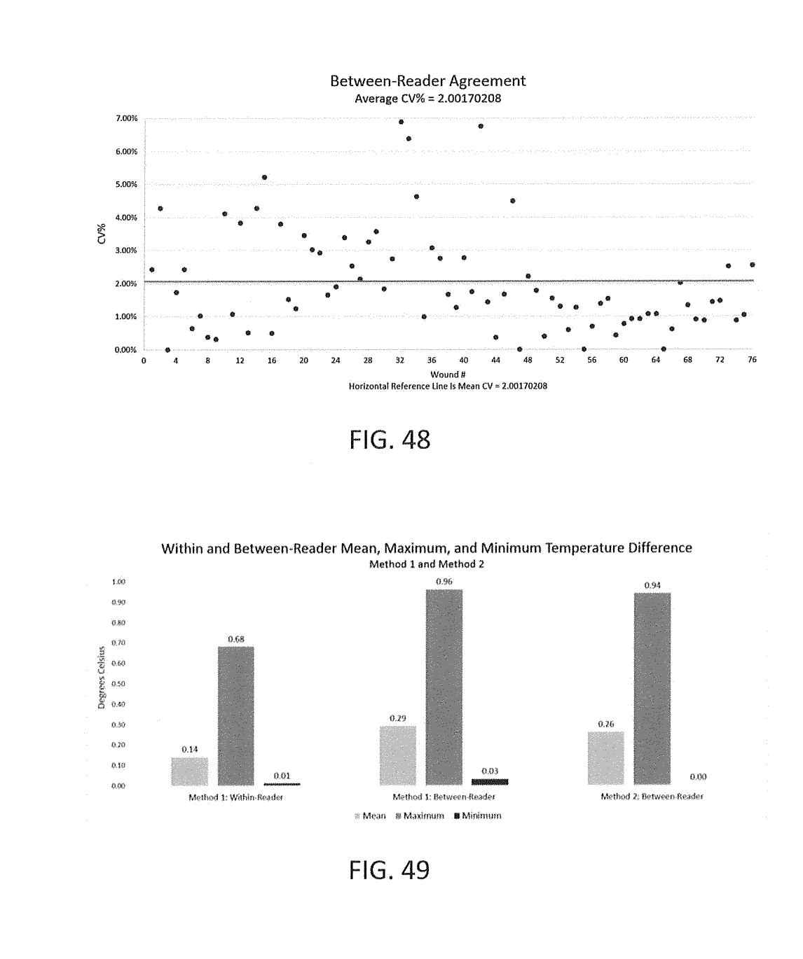

When provided a reference point on the initial image, there was no significant difference observed in the performance between readers across all 76 wound images. The between-reader coefficient of variation (CV) for Mean Temperature was approximately 2% (FIG. 48). When examined individually, the minimum between-reader percent coefficient of variation was 0.00, the maximum between-reader percent coefficient of variation was 6.88.

RE FIG. 48: Between-reader percent coefficient of variation for Mean Temperature averaged across all three readers are shown.

The overall average difference in Mean Temperature between-readers is 3.29 Pixel Values (or 0.26° C.). The minimum observed Mean Temperature difference is 0 Pixel Values (or 0.00° C.) and the maximum is 12 Pixel Values (or 0.96° C.). The Mean Temperature variation is similar to the within-reader and between-reader differences observed in Method 1. By providing a reference point initially, the variability between readers is reduced with the average Mean Temperature variation across all 76 images of approximately 0.25° C.

Within- and Between-Reader Agreement of Initial Patient Encounter Images:

The control area measurements were found to be very consistent both within and between-readers. The within-reader variability for Mean Temperature is low with a percent coefficient of variation of approximately 1%. The between-reader variation for Mean Temperature was also good with a percent coefficient of variation of approximately 2%. The average Maximum Temperature had a coefficient of variation within-reader of 1.14% and between-reader of 1.97%. The average Minimum Temperature had a within-reader coefficient of variation of 1.10% and a between-reader coefficient of variation of 02.01%.

The within and between-reader average difference in Mean Temperature was 0.14° C. and 0.29° C., respectively. The largest Mean Temperature Difference observed within-readers was 0.68° C., with the smallest difference being 0.01° C. For between-reader Mean Temperature Difference, the largest difference observed was 0.96° C., with the smallest difference being 0.03° C. (FIG. 49).

RE FIG. 49: Within and between-reader Average, Maximum, and Minimum Difference in Mean Temperature for Methods 1 and 2 (both methods assessed independently) are shown.

The results from Method 1 (within- and between-reader agreement) demonstrate that control area selection may be delineated repeatedly by the same operator and reproducibly by different operators. Thus, clinicians can utilize relative temperature differential as a reliable measurement when using long-wave infrared thermography for a physiological assessment of tissue or wounds in order to extrapolate data relating to presence or absence of blood flow, perfusion, and metabolic activity in the wound, periwound, and wound site on an initial patient encounter.

Between-Reader Agreement of Follow-Up Encounter Images:

When provided an initial control area, longitudinal selection of subsequent control areas were found to be extremely consistent between readers. The between-reader variability for Mean Temperature was low with the coefficient of variation approximately 2% with an average difference in Mean Temperature average of approximately 0.26° C. The largest Mean Temperature Difference observed between-readers was 0.94° C., with the smallest difference being 0.00° C. (or no difference at all) (FIG. 49). When assessing for a difference between readers, there were no statistically significant differences observed (p>0.91).

The results from Method 2 (between-reader agreement) demonstrate that when provided a view of the same control area selection from a previous encounter, different operators can reproducibly select the same area as a control. As a result of this, clinicians can reliably compare longitudinal changes in relative temperature differential through the use of long-wave infrared thermography. The physiological changes, as represented by relative temperature, are then able to be integrated as an adjunctive tool to aid clinicians in their decisions as it relates to optimal care plans, treatment, and interventions for wounds and wound prevention.

Study Conclusions:

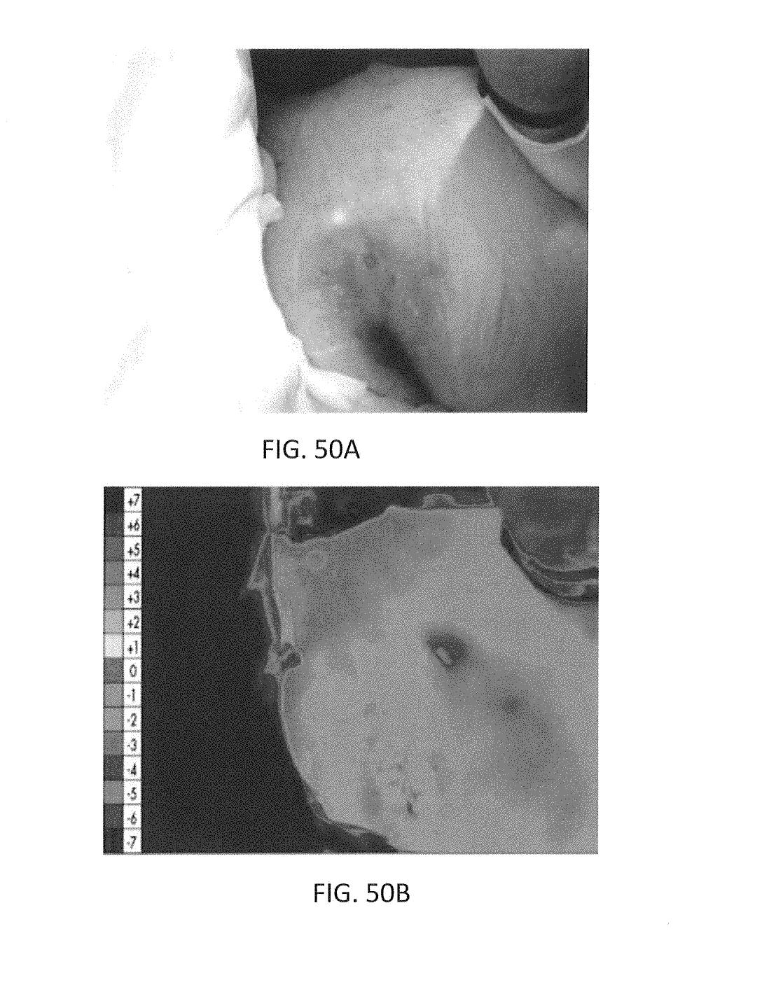

With repeatable and reliable relative temperature data clinicians are able to compare the parities and disparities between the “healthy/good” and the “unhealthy/bad” tissues to enhance their ability to quantitatively measure and compare of an area of interest's progression or regression. For example, a single snapshot of relative temperature data could provide valuable clinical insight such as the revelation of a suspected subcutaneous tissue aberration not visually present. Also, measuring and comparing an existing open wound over time can assist clinicians to better understand the pathophysiologic principles of the healing processes. This study demonstrates that clinicians can repeatably and reliably perform a relative temperature differential/RTD analysis. This enables the clinician to more easily and promptly determine if there exists a formation of tissue with similar structures and comparable functions to that of the unaffected control area or if there exists formation of tissue that is structurally and functionally satisfactory but not identical to that of the unaffected control area. Measuring relative temperature difference enables the clinician to complete a skin assessment that yields information beyond what the International Guidelines recommend. The images in FIGS. 50A, 50B; 51A, 51B; 52A, 52B; 53A, 53B; 54A, 54B; 55A, 55B; 56A, 56B; 57A, 57B; and 58A, 58b provide examples of four different scenarios where using LWIT to assess temperature can aid in the assessment of the skin and underlying tissue and other wounds.

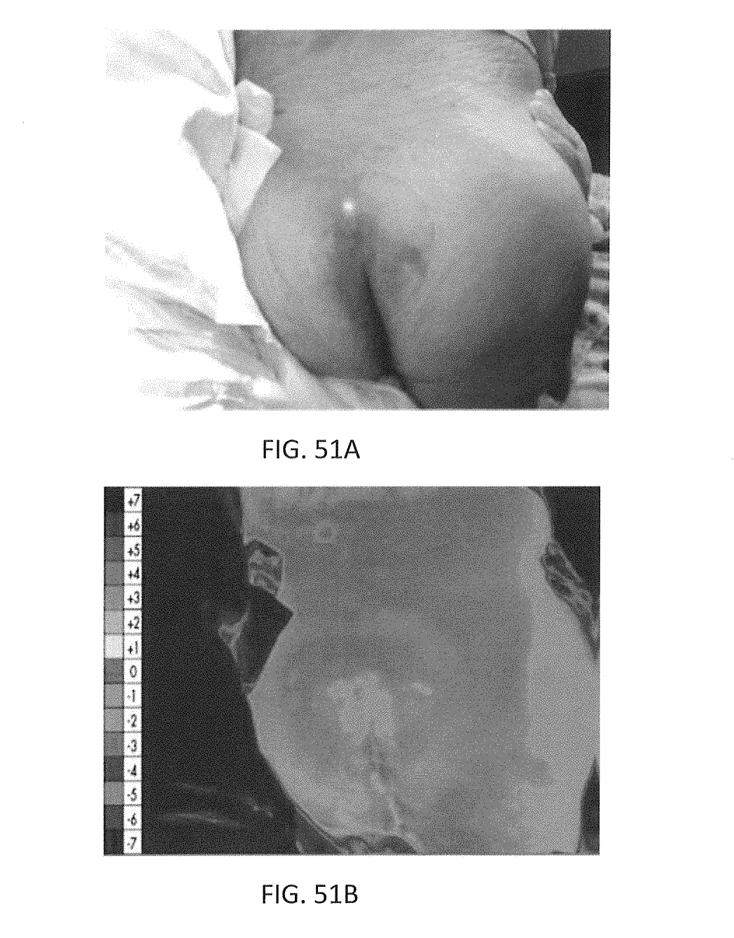

FIGS. 50A to 51B relate to a Suspected Deep Tissue Injury scenario. The image set on FIGS. 50A and 50B represents a non-visible suspected deep tissue injury captured present on admission. After recognizing and documenting, the image set of FIGS. 51A and 51B shows the success of the intervention to mitigate the progression to a full-thickness pressure ulcer. Prior studies suggest that temperature measurement can assist in the detection of underlying skin necrosis and as an objective, non-invasive and quantitative means of early DTI diagnosis.

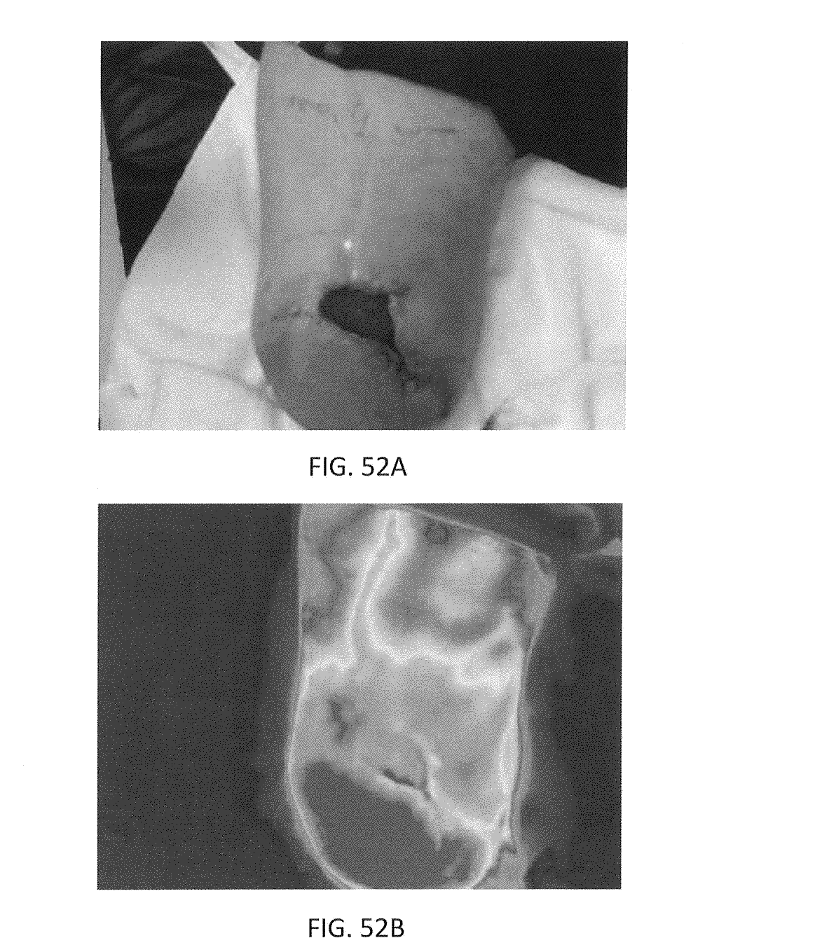

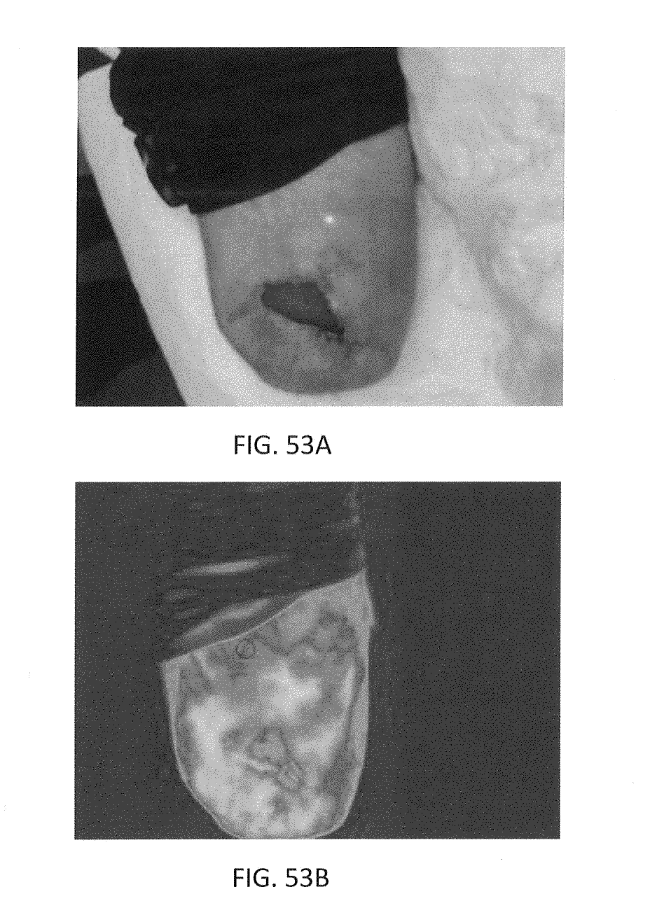

FIGS. 52A to 53B relate to a Surgical Site Infection scenario. This pair of image sets represent a surgical site infection with abscess. The pre-treatment RTD image of FIG. 52B reveals a strong increase in heat prior to intervention. The image set of FIGS. 53A and 53B confirms the positive response from an incision and drainage of the abscess and antibiotic therapy. Thermography as a tool for physiological assessment of the skin and underlying tissue is supported by a number of prior studies which suggest incorporating quantitative skin temperature measurement into routine wound assessment provides a timely and reliable method to quantify the heat associated with deep and surrounding skin infection and to monitor ongoing wound status, a useful predictor wound healing and assessment of the presence of critical colonisation or other factors which disturb the wound healing, as well as useful tool for screening for osteomyelitis in patients with diabetic feet.

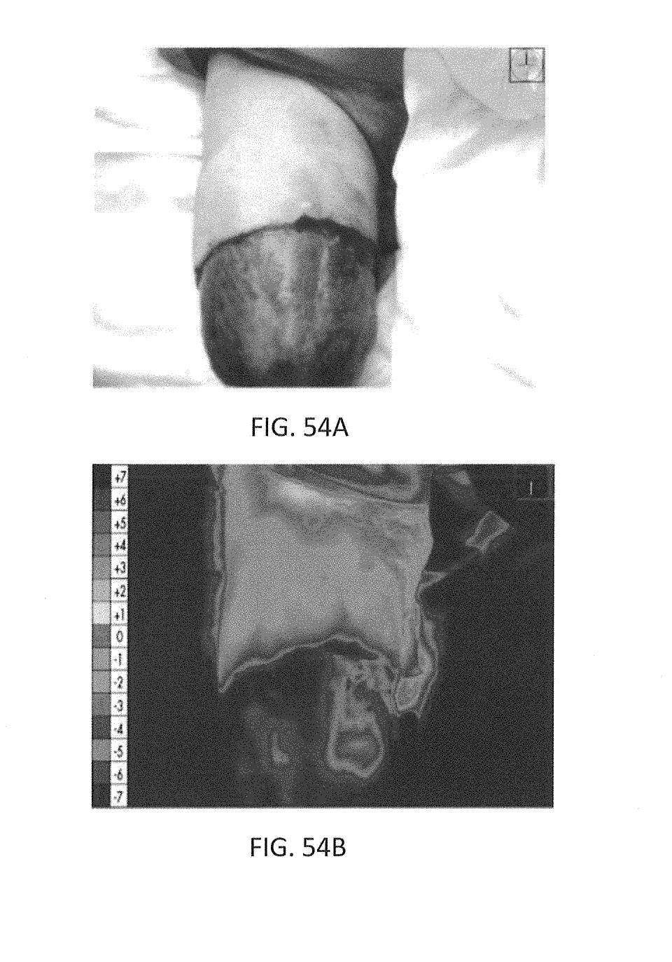

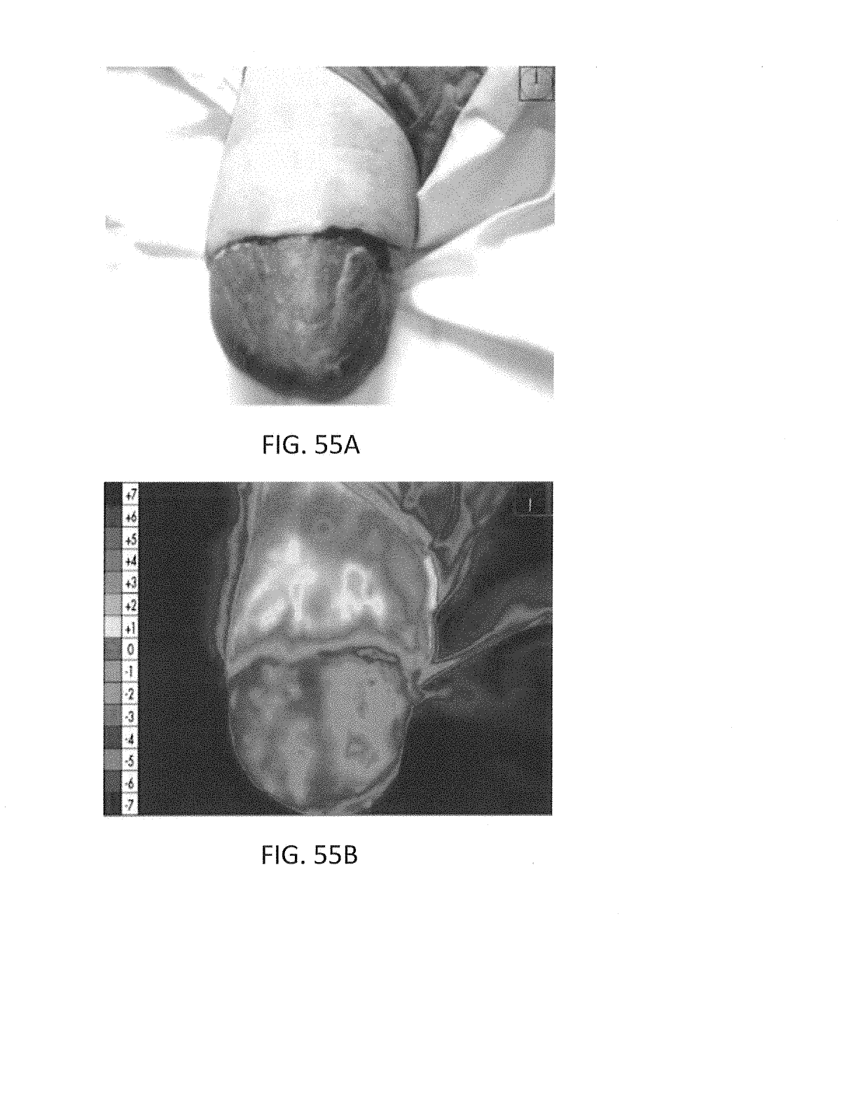

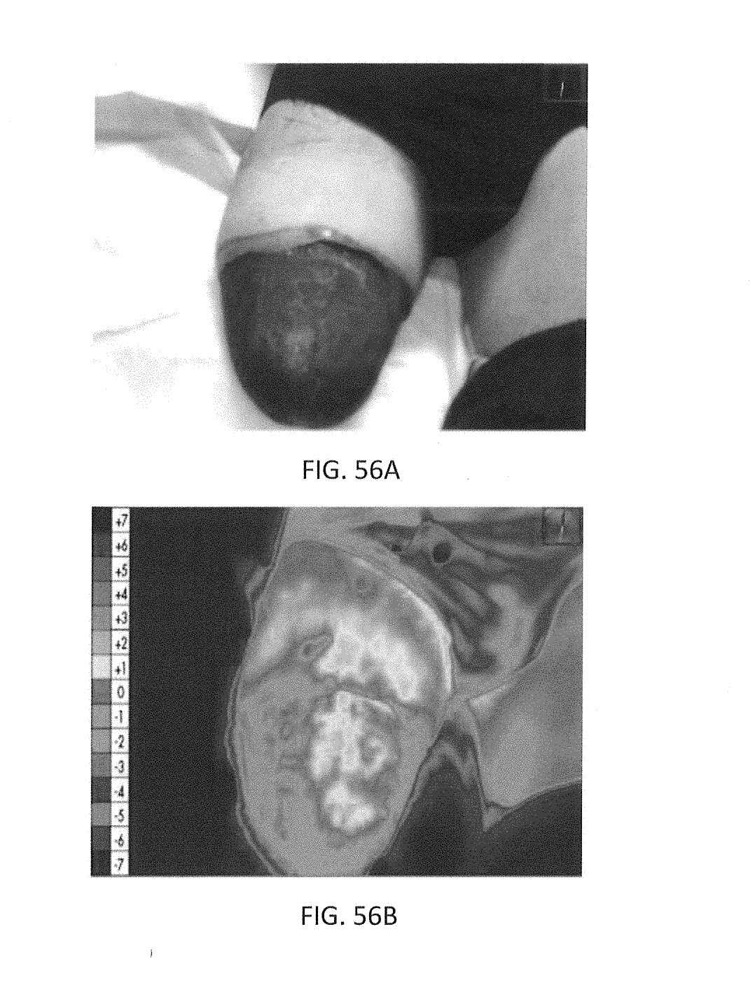

FIGS. 54A to 56B relate to an Objective Wound Assessment. The longitudinal image series represents an amputation as a result of a crush injury. The RTD images of FIGS. 54B, 55B, and 56B allowed for the objective assessment of the chosen therapy, negative pressure wound therapy (NPWT). In this example, clinicians were able to objectively identify that the chosen treatment was providing the proper physiological response, revascularization. The revascularization seen here causes perfusion and metabolic activity, ultimately increasing temperature. This increase in temperature is manifested in the form of inflammation. Conversely, when there is no vascularization there can be no perfusion and metabolic activity which ultimately results in a decrease in temperature. The decrease in temperature is manifested in the form of inadequate tissue perfusion or in some situations ischemia.

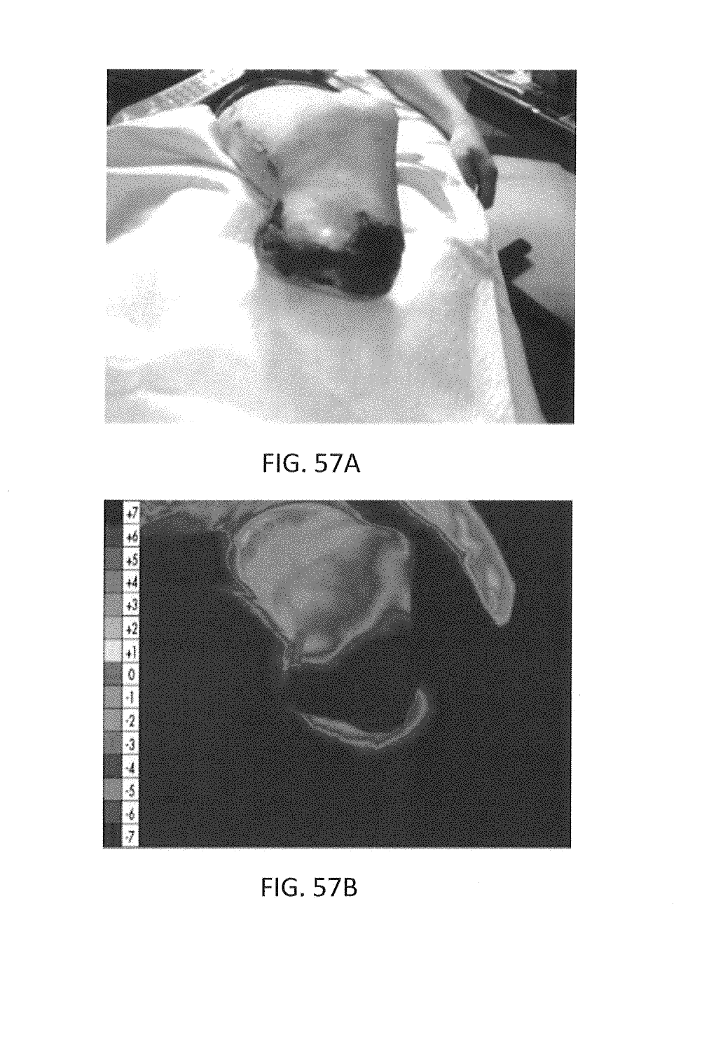

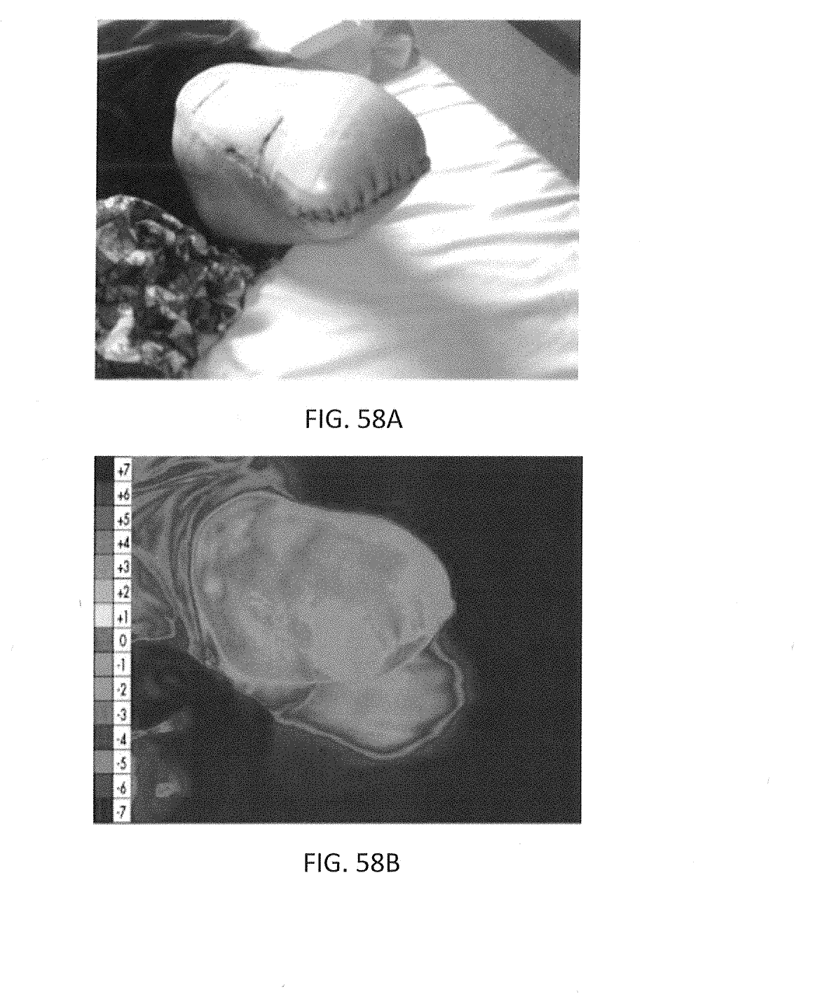

FIGS. 57A to 58B relate to a Limb Salvage scenario. This pair of image sets represent an image set of an extremity after a below and above-the-knee amputation (BKA and AKA). Prior to the initial BKA, the physician had strongly recommended beginning with an AKA. The RTD image of FIG. 57B aligns with the recommendation as it reveals a strong decrease in lower extremity circulation. After surgical revision to an AKA, the image set of FIGS. 58A and 58B shows improvement in circulation and further confirms that an initial AKA would have been optimal choice. Prior studies demonstrate that the thermographic method is a reliable indicator of the level of a major limb amputation.

The thermal energy of a body surface is a reflection of the presence or absence of perfusion of the dermal and subcutaneous tissues. Since tests of adequate perfusion are a common part of the patient assessment process, clinicians may use long-wave infrared thermography/LWIT to measure hyperperfusion (increased blood flow) and hypoperfusion (decreased blood flow) of skin and subcutaneous tissue. This will enable the identification of aberrations and/or existing open wounds relative to the average level of perfusion of an unaffected, adjacent body surface (parities and disparities between the good and the bad). This can be incorporated with other common methods of perfusion evaluation including skin color, patient condition and capillary refill. Thus, when comparing a compromised area to an uncompromised area the clinician may select a regional, adjacent area of intact tissue as a control and comparator for baseline body surface temperature measurement. This data can be used to repeatably and reliably assess and simulate the impact of the physiological parities and disparities of existing wounds and suspected wounds.

M. A System, Apparatus, and Method for Capturing a Combination 3D, Thermal, and 2D Image:

An embodiment of the present invention provides an image capturing device system adapted to find depths in a target from a distance of about 1 to 4 meters. The exemplary system embodiment includes a USB 3.0 peripheral device including a module (such as a PCB board with components and a reinforcing frame) enclosable within a housing. The device further includes a 3D camera of known, commercially available type such as, for example, RealSense™ 3D camera manufactured by Intel®, non-limiting examples of which include the D400-Series, ZR300, SR300, or R200 RealSense™ 3D cameras, published descriptions of which are available at https://software.intel.com/en-us/realsense/ and are incorporated herein by reference.

An image capturing device in accordance with the present invention includes an HD quality visual camera to provide a color still image or image stream; two stereo aligned near wave infrared cameras used for generating scene depth data; and a near wave infrared laser projector, to augment low texture scenes (like flat surfaces) for improved depth measurements.

The combination of stereo cameras and infrared laser projector make up the depth capability of the hardware. Intel, for example, provides a PC side software developer kit (SDK) capable of constructing 3D meshes (contour maps) and color texture overlays from the output of the camera system.

The single integrated camera apparatus of the present invention, however, further comprises a long wave infrared camera, such as one from DRS Technologies, capable of sensing thermal features beneath human surface tissues that the near wave infrared camera cannot detect.

The inventive embodiment further comprises software means for integrating and fusing data from all visualization sources into an efficient real time output capable of capturing, reporting, and displaying clinically relevant wound/feature measurements from all camera sources and storing this data for recall and clinical review.

The software and hardware combination of the present invention provides accurate tissue surface measurements based upon depth data across a predetermined operational range. This allows a user to increase or decrease the field of view of the wound area as required.

The system and apparatus of the present invention further comprises means to provide depth measurements of the interior of wounds. This includes depth at user selected points and automatically finding the deepest point in the target wound.

The system and apparatus of the present invention further comprises means to trace the perimeter of a visual wound or long wave thermal feature once a user identifies the target wound or thermal feature by mouse click or tapping a touch screen. The user may need to occasionally adjust contour selection thresholds.

The system and apparatus of the present invention further comprises means to document wound size and shape as a three dimensional mesh (surface contour map). A specific limitation of 2D visual clinical images is their inability to see behind the curve of a limb. If a wound wraps around a limb or other body surface, 2D technology cannot provide accurate measurements of wound perimeter or depth. Using stitched together 3D meshes the present invention accurately maps and measures the entire wound surface even if it wraps around a limb.

The present invention software further creates and stores these 3D meshes for clinical documentation and evaluation in real-time or later.

The system and/or device embodiment includes software means for fusing 3D mesh, color image, and the location of any long wave infrared thermal features, into a single clinical view of a wound site, providing an image that combines the depth information in layers.

Thus, the present invention software combines the outputs of two camera modules into a single view (a fused view) of a wound site, for documentation and measurement evaluation, which neither camera module can completely provide.

One exemplary embodiment thus provides a combination thermal and visual image capturing device to capture real time thermal and visual images of surface and subsurface biological tissue. The device is a USB peripheral device including a power source, a long wave infrared microbolometer, a short wave infrared microbolometer, a 3D camera, and a digital (i.e., 2D) camera, each functionally connected to the power source, with the 3D and digital cameras contained within a device housing.

The device includes means for electronically providing combined thermal image information from the microbolometers and visual image information from the digital and the 3D cameras to another electronic device or system.

Another exemplary embodiment thus provides a combination thermal and visual image capturing system used to capture, store, and report combined 2D, 3D, thermal and visual images of surface and subsurface biological tissue. The system includes an image capturing device such as described above. The device includes means for combining image data into a single or layered visual image; and means for electronically displaying or storing combined thermal image information from the microbolometers and visual image information from the digital and 3D cameras.

A method for capturing and combining a long wave infrared image, a short wave infrared image, a 3D image, and a 2D image into a single fused image includes the steps of: obtaining a short wave infrared image; obtaining a long wave infrared image; obtaining a 2D color image; and combining the images into a single fused 3D image.

While this invention has been described with respect to example embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A combination thermal and visual image capturing USB peripheral device adapted to capture real time thermal and visual images of surface and subsurface biological tissue, comprising:
a power source;
a long wave infrared microbolometer functionally connected to the power source;
a short wave infrared microbolometer functionally connected to the power source;
a 3D camera functionally connected to the power source;
a digital camera functionally connected to the power source;
a housing, the 3D and digital cameras contained within the housing; and
means for electronically providing combined thermal image information from the microbolometers and visual image information from digital camera and the 3D camera to another electronic device or system.

2. A combination thermal and visual image capturing system used to capture, store, and report combined 2D, 3D, thermal and visual images of surface and subsurface biological tissue, comprising:
an image capturing device that is a USB peripheral device, comprising:
a power source;
a long wave infrared microbolometer functionally connected to the power source;
a short wave infrared microbolometer functionally connected to the power source;
a digital camera functionally connected to the power source;
a 3D camera functionally connected to the power source;
a housing, the digital and 3D cameras contained within the housing;
means for combining image data into a single or layered visual image; and
means for electronically displaying or storing combined thermal image information from the microbolometers and visual image information from the digital 3D cameras.

3. A method for capturing and combining a long wave infrared image, a short wave infrared image, a 3D image, and a 2D image into a single fused image, comprising the steps of:
obtaining a short wave infrared image;
obtaining a long wave infrared image;
obtaining a 2D color image; and
combining the images into a single fused 3D image.
